(19) United States
(12) Patent Application Publication
(10) Pub. No.: US 2016/0135739 A1
(43) Pub. Date: May 19, 2016

(54) DETERMINING TREATMENT COMPLIANCE USING COMBINED PERFORMANCE INDICATORS

(71) Applicant: Elwha LLC, Bellevue, WA (US)

(72) Inventors: Jeffrey A. Bowers, Bellevue, WA (US); Paul Duesterhoft, Grapevine, TX (US); Daniel Hawkins, Pleasanton, CA (US); Roderick A. Hyde, Redmond, WA (US); Edward K.Y. Jung, Bellevue, WA (US); Jordin T. Kare, San Jose, CA (US); Eric C. Leuthardt, St. Louis, MO (US); Nathan P. Myhrvold, Medina, WA (US); Michael A. Smith, Phoenix, AZ (US); Elizabeth A. Sweeney, Seattle, WA (US); Clarence T. Tegreene, Mercer Island, WA (US); Lowell L. Wood, JR., Bellevue, WA (US); Victoria Y.H. Wood, Livermore, CA (US)

(73) Assignee: Elwha LLC, a limited liability company of the State of Delaware

(21) Appl. No.: 14/939,143

(22) Filed: Nov. 12, 2015

Related U.S. Application Data
(63) Continuation-in-part of application No. 14/543,030, filed on Nov. 17, 2014.
Application No. 14/543,030 is a continuation-in-part of application No. 14/543,066, filed on Nov. 17, 2014.
Application No. 14/543,066 is a continuation-in-part of application No. 14/729,278, filed on Jun. 3, 2015.
Application No. 14/729,278 is a continuation-in-part of application No. 14/729,322, filed on Jun. 3, 2015.

(51) Int. Cl. A61B 5/00; A61B 5/16; A61B 5/1171; A61B 5/0205; A61B 7/00

(52) U.S. Cl. CPC A61B 5/4833; A61B 5/0205; A61B 5/4803; A61B 7/00; A61B 5/1176; A61B 5/72; A61B 5/746; A61B 5/0002; A61B 5/7405; A61B 5/167; A61B 5/4082; A61B 5/4088; A61B 5/165

(57) ABSTRACT

Methods and systems for monitoring compliance of a patient with a prescribed treatment regimen are described. Patient activity is detected unobtrusively with an activity sensor at the patient location, and activity data is transmitted to a monitoring location. Patient speech detected during use of a communication system such as a mobile telephone by the patient may also be used as an activity signal. Patient activity and/or speech is processed at the patient location or monitoring location to identify activity parameters or patterns that indicate whether the patient has complied with the prescribed treatment regimen. The activity sensor and other components at the patient location may be incorporated into, or associated with, a cell phone, computing system, game system, or vehicle system, for example. The system may provide a report to an interested party, for example a medical care provider or insurance company, regarding patient compliance with the prescribed treatment regimen.

If an Application Data Sheet (ADS) has been filed on the filing date of this application, it is incorporated by reference herein. Any applications claimed on the ADS for priority under 35 U.S.C. §§119, 120, 121, or 365(c), and any and all parent, grandparent, great-grandparent, etc. applications of such applications, are also incorporated by reference, including any priority claims made in those applications and any material incorporated by reference, to the extent such subject matter is not inconsistent herewith.

### CROSS-REFERENCE TO RELATED APPLICATIONS

The present application claims the benefit of the earliest available effective filing date(s) from the following listed application(s) (the “Priority Applications”), if any, listed below (e.g., claims earliest available priority dates for other than provisional patent applications or claims benefits under 35 USC §119(e) for provisional patent applications, for any and all parent, grandparent, great-grandparent, etc. applications of the Priority Application(s)).

### PRIORITY APPLICATIONS

The present application constitutes a continuation-in-part of U.S. patent application Ser. No. 14/543,030, entitled MONITORING TREATMENT COMPLIANCE USING SPEECH PATTERNS PASSIVELY CAPTURED FROM A PATIENT ENVIRONMENT, naming Jeffrey A. Bowers, Paul Duesterhoft, Daniel Hawkins, Roderick A. Hyde, Edward K. Y. Jung, Jordin T. Kare, Eric C. Leuthardt, Nathan P. Myhrvold, Michael A. Smith, Elizabeth A. Sweeney, Clarence T. Tegreene, and Lowell L. Wood, Jr. as inventors, filed 17 Nov. 2014 with attorney docket no. 0810-004-006-000000, which is currently co-pending or is an application of which a currently co-pending application is entitled to the benefit of the filing date.

The present application constitutes a continuation-in-part of U.S. patent application Ser. No. 14/543,066, entitled DETERMINING TREATMENT COMPLIANCE USING SPEECH PATTERNS PASSIVELY CAPTURED FROM A PATIENT ENVIRONMENT, naming Jeffrey A. Bowers, Paul Duesterhoft, Daniel Hawkins, Roderick A. Hyde, Edward K. Y. Jung, Jordin T. Kare, Eric C. Leuthardt, Nathan P. Myhrvold, Michael A. Smith, Elizabeth A. Sweeney, Clarence T. Tegreene, and Lowell L. Wood, Jr. as inventors, filed 17 Nov. 2014 with attorney docket no. 0810-004-007-000000, which is currently co-pending or is an application of which a currently co-pending application is entitled to the benefit of the filing date.

The present application constitutes a continuation-in-part of U.S. patent application Ser. No. 14/729,278, entitled MONITORING TREATMENT COMPLIANCE USING SPEECH PATTERNS CAPTURED DURING USE OF A COMMUNICATION SYSTEM, naming Jeffrey A. Bowers, Paul Duesterhoft, Daniel Hawkins, Roderick A. Hyde, Edward K. Y. Jung, Jordin T. Kare, Eric C. Leuthardt, Nathan P. Myhrvold, Michael A. Smith, Elizabeth A. Sweeney, Clarence T. Tegreene, and Lowell L. Wood, Jr. as inventors, filed 3 Jun. 2015 with attorney docket no. 0810-004-008-000000, which is currently co-pending or is an application of which a currently co-pending application is entitled to the benefit of the filing date.

The present application constitutes a continuation-in-part of U.S. patent application Ser. No. 14/729,322, entitled DETERMINING TREATMENT COMPLIANCE USING SPEECH PATTERNS CAPTURED DURING USE OF A COMMUNICATION SYSTEM, naming Jeffrey A. Bowers, Paul Duesterhoft, Daniel Hawkins, Roderick A. Hyde, Edward K. Y. Jung, Jordin T. Kare, Eric C. Leuthardt, Nathan P. Myhrvold, Michael A. Smith, Elizabeth A. Sweeney, Clarence T. Tegreene, and Lowell L. Wood, Jr. as inventors, filed 3 Jun. 2015 with attorney docket no. 0810-004-009-000000, which is currently co-pending or is an application of which a currently co-pending application is entitled to the benefit of the filing date.

If the listings of applications provided above are inconsistent with the listings provided via an ADS, it is the intent of the Applicant to claim priority to each application that appears in the Domestic Benefit/National Stage Information section of the ADS and to each application that appears in the Priority Applications section of this application.

All subject matter of the Priority Applications and of any and all applications related to the Priority Applications by priority claims (directly or indirectly), including any priority claims made and subject matter incorporated by reference therein as of the filing date of the instant application, is incorporated herein by reference to the extent such subject matter is not inconsistent herewith.

### SUMMARY

In an aspect, a system includes, but is not limited to, at least one receiving device for use at a monitoring location for receiving an activity data signal transmitted to the monitoring location from a patient location, the activity data signal containing activity data representing at least one non-speech activity pattern in activity sensed from a patient with at least one activity sensor in an unobtrusive activity-detection system at the patient location during performance of the non-speech activity by the patient, the patient having a brain-related disorder and a prescribed treatment regimen for treating at least one aspect of the brain-related disorder, signal processing circuitry configured to analyze the activity data signal to determine whether the activity data represents at least one non-speech activity pattern that matches at least one characteristic activity pattern, compliance determination circuitry configured to determine whether the patient has complied with the prescribed treatment regimen based upon whether the activity data represents the non-speech activity pattern that matches the at least one characteristic activity pattern, and reporting circuitry configured to report a conclusion based on the determination of whether the patient has complied with the prescribed treatment regimen. In addition to the foregoing, other system aspects are described in the claims, drawings, and text forming a part of the disclosure set forth herein.

In an aspect, a method of monitoring compliance of a patient with a prescribed treatment regimen includes, but is not limited to, receiving an activity data signal with a receiving device at a monitoring location, the activity data signal transmitted to the monitoring location from a patient location, the activity data signal containing activity data representing at least one non-speech activity pattern in activity sensed from a patient with at least one activity sensor in an unobtrusive activity-detection system at the patient location during performance of the non-speech activity by the patient, the patient having a brain-related disorder and a prescribed treatment regimen intended to treat at least one aspect of the brain-related disorder, analyzing the activity data signal with signal processing circuitry at the monitoring location to determine whether the activity data represents at least one non-speech activity pattern that matches at least one characteristic activity pattern, determining with compliance determination circuitry at the monitoring location whether the patient has complied with the prescribed treatment regimen based on whether the activity data represents the at least one non-speech activity pattern that matches the at least one characteristic activity pattern, and reporting with reporting circuitry a conclusion based on the determination of whether the patient has complied with the prescribed treatment regimen. In addition to the foregoing, other method aspects are described in the claims, drawings, and text forming a part of the disclosure set forth herein.

In an aspect, a computer program product includes, but is not limited to, a non-transitory signal-bearing medium bearing one or more instructions for receiving an activity data signal with a receiving device at a monitoring location, the activity data signal transmitted to the monitoring location from a patient location, the activity data signal containing activity data representing at least one non-speech activity pattern in activity sensed from a patient with at least one activity sensor in an unobtrusive activity-detection system at the patient location during performance of the non-speech activity by the patient, the patient having a brain-related disorder and a prescribed treatment regimen intended to treat at least one aspect of the brain-related disorder, one or more instructions for analyzing the activity data signal with signal processing circuitry at the monitoring location to determine whether the activity data represents at least one non-speech activity pattern that matches at least one characteristic activity pattern, one or more instructions for determining with compliance determination circuitry at the monitoring location whether the patient has complied with the prescribed treatment regimen based on whether the activity data represents the at least one non-speech activity pattern that matches the at least one characteristic activity pattern, and one or more instructions for reporting with reporting circuitry a conclusion based on the determination of whether the patient has complied with the prescribed treatment regimen. In addition to the foregoing, other aspects of a computer program product including one or more non-transitory machine-readable data storage media bearing one or more instructions are described in the claims, drawings, and text forming a part of the disclosure set forth herein.

In an aspect, a system includes, but is not limited to a computing device, and instructions that when executed on the computing device cause the computing device to control the receiving of an activity data signal with a receiving device at a monitoring location, the activity data signal transmitted to the monitoring location from a patient location, the activity data signal containing activity data representing at least one non-speech activity pattern in activity sensed from a patient with at least one activity sensor in an unobtrusive activity-detection system at the patient location during performance of the non-speech activity by the patient, the patient having a brain-related disorder and a prescribed treatment regimen intended to treat at least one aspect of the brain-related disorder, analyze the activity data signal with signal processing circuitry at the monitoring location to determine whether the activity data represents at least one non-speech activity pattern that matches at least one characteristic activity pattern, determine with compliance determination circuitry at the monitoring location whether the patient has complied with the prescribed treatment regimen based on whether the activity data represents the at least one non-speech activity pattern that matches the at least one characteristic activity pattern, and control the reporting with reporting circuitry of a conclusion based on the determination of whether the patient has complied with the prescribed treatment regimen. In addition to the foregoing, other system aspects are described in the claims, drawings, and text forming a part of the disclosure set forth herein.

In an aspect, an unobtrusive activity-detection system includes, but is not limited to, at least one activity sensor for sensing at least one activity signal including a non-speech activity pattern corresponding to performance of a non-speech activity by a patient at a patient location, the patient having a brain-related disorder and a prescribed treatment regimen for treating at least one aspect of the brain-related disorder, activity detection circuitry configured to identify at least one section of the at least one activity signal containing the non-speech activity pattern, activity analysis circuitry for processing the at least one section of the at least one activity signal to generate activity data including data indicative of whether the patient has complied with the treatment regimen, and at least one transmitting device for transmitting an activity data signal including the activity data including data indicative of whether the patient has complied with the treatment regimen from the patient location to a receiving device at a monitoring location. In addition to the foregoing, other system aspects are described in the claims, drawings, and text forming a part of the disclosure set forth herein.

In an aspect, a method includes, but is not limited to, sensing with at least one activity sensor in an unobtrusive activity-detection system at least one activity signal including a non-speech activity pattern corresponding to performance of a non-speech activity by a patient at a patient location, the patient having a brain-related disorder and a prescribed treatment regimen for treating at least one aspect of the brain-related disorder, processing the at least one activity signal with activity detection circuitry in the unobtrusive activity-detection system to identify at least one section of the at least one activity signal containing the non-speech activity pattern, analyzing the at least one section of the at least one activity signal with activity analysis circuitry in the unobtrusive activity-detection system to generate activity data including data indicative of whether the patient has complied with the treatment regimen, and transmitting an activity data signal including the activity data including data indicative of whether the patient has complied with the treatment regimen to a receiving device at a monitoring location with at least one transmitting device at the patient location. In addition to the foregoing, other method aspects are described in the claims, drawings, and text forming a part of the disclosure set forth herein.

In an aspect, a computer program product includes, but is not limited to, a non-transitory signal-bearing medium bearing one or more instructions for sensing with at least one activity sensor in an unobtrusive activity-detection system at least one activity signal including a non-speech activity pattern corresponding to performance of a non-speech activity by a patient at a patient location, the patient having a brain-related disorder and a prescribed treatment regimen for treating at least one aspect of the brain-related disorder, one or more instructions for processing the at least one activity signal with activity detection circuitry in the unobtrusive activity-detection system to identify at least one section of the at least one activity signal containing the non-speech activity pattern, one or more instructions for analyzing the at least one section of the at least one activity signal with activity analysis circuitry in the unobtrusive activity-detection system to generate activity data including data indicative of whether the patient has complied with the treatment regimen, and one or more instructions for transmitting an activity data signal including the activity data including data indicative of whether the patient has complied with the treatment regimen to a receiving device at a monitoring location with at least one transmitting device at the patient location. In addition to the foregoing, other aspects of a computer program product including one or more non-transitory machine-readable data storage media bearing one or more instructions are described in the claims, drawings, and text forming a part of the disclosure set forth herein.

In an aspect, a system includes, but is not limited to, a computing device and instructions that when executed on the computing device cause the computing device to control the sensing with at least one activity sensor in an unobtrusive activity-detection system of at least one activity signal including a non-speech activity pattern corresponding to performance of a non-speech activity by a patient at a patient location, the patient having a brain-related disorder and a prescribed treatment regimen for treating at least one aspect of the brain-related disorder, process the at least one activity signal with activity detection circuitry in the unobtrusive activity-detection system to identify at least one section of the at least one activity signal containing the non-speech activity pattern, analyze the at least one section of the at least one activity signal with activity analysis circuitry in the unobtrusive activity-detection system to generate activity data including data indicative of whether the patient has complied with the treatment regimen, and control the transmitting of an activity data signal including the activity data including data indicative of whether the patient has complied with the treatment regimen to a receiving device at a monitoring location with at least one transmitting device at the patient location. In addition to the foregoing, other system aspects are described in the claims, drawings, and text forming a part of the disclosure set forth herein.

In an aspect, a system includes, but is not limited to, at least one receiving device for use at a monitoring location for receiving at least one activity data signal and at least one audio data signal from a communication system, the at least one audio data signal including audio data representing speech from a patient at a patient location sensed with at least one audio sensor at the patient location during use of the communication system and transmitted to the monitoring location, the patient having a brain-related disorder and a prescribed treatment regimen for treating at least one aspect of the brain-related disorder, the at least one activity data signal including activity data indicative of whether the patient has complied with the prescribed treatment regimen, the activity data representing at least one first activity of the patient, signal processing circuitry configured to process the at least one activity data signal to determine based upon the at least one first activity of the patient and at least one second activity of the patient whether the patient has complied with the prescribed treatment regimen, and reporting circuitry configured to report a conclusion based on the determination of whether the patient has complied with the prescribed treatment regimen. In addition to the foregoing, other system aspects are described in the claims, drawings, and text forming a part of the disclosure set forth herein.

In an aspect, a method of monitoring compliance of a patient with a treatment regimen includes, but is not limited to receiving at least one audio data signal with a receiving device at a monitoring location, the audio data signal including audio data representing speech sensed from a patient at a patient location during use of a communication system, the patient having a brain-related disorder and a prescribed treatment regimen for treating at least one aspect of the brain-related disorder, receiving at least one activity data signal with the receiving device, the activity data signal including activity data indicative of whether the patient has complied with the treatment regimen, the activity data representing at least one first activity of the patient, determining with signal processing circuitry at the monitoring location whether the patient has complied with the treatment regimen, based upon the at least one first activity of the patient and upon at least one second activity of the patient, and reporting with reporting circuitry a conclusion based on the determination of whether the patient has complied with the prescribed treatment regimen. In addition to the foregoing, other method aspects are described in the claims, drawings, and text forming a part of the disclosure set forth herein.

In an aspect, a computer program product includes, but is not limited to, a non-transitory signal-bearing medium bearing one or more instructions for controlling the receiving of at least one audio data signal with a receiving device at a monitoring location, the audio data signal including audio data representing speech sensed from a patient at a patient location during use of a communication system, the patient having a brain-related disorder and a prescribed treatment regimen for treating at least one aspect of the brain-related disorder, one or more instructions for controlling the receiving of at least one activity data signal with the receiving device, the activity data signal including activity data indicative of whether the patient has complied with the treatment regimen, the activity data representing at least one first activity of the patient, one or more instructions for determining whether the patient has complied with the treatment regimen, based upon the at least one first activity of the patient and upon at least one second activity of the patient, and one or more instructions for controlling reporting circuitry to report a conclusion based on the determination of whether the patient has complied with the prescribed treatment regimen. In addition to the foregoing, other aspects of a computer program product including one or more non-transitory machine-readable data storage media bearing one or more instructions are described in the claims, drawings, and text forming a part of the disclosure set forth herein.

In an aspect, a system includes, but is not limited to, a computing device, and instructions that when executed on the computing device cause the computing device to control the receiving of at least one audio data signal with a receiving device at a monitoring location, the audio data signal including audio data representing speech sensed from a patient at a patient location during use of a communication system, the patient having a brain-related disorder and a prescribed treatment regimen for treating at least one aspect of the brain-related disorder, control the receiving of at least one activity data signal with the receiving device, the activity data signal including activity data indicative of whether the patient has complied with the treatment regimen, the activity data representing at least one first activity of the patient, determining whether the patient has complied with the treatment regimen, based upon the at least one first activity of the patient and upon at least one second activity of the patient, and controlling reporting circuitry to report a conclusion based on the determination of whether the patient has complied with the prescribed treatment regimen. In addition to the foregoing, other system aspects are described in the claims, drawings, and text forming a part of the disclosure set forth herein.

In an aspect, a system includes, but is not limited to, at least one audio sensor in a communication system for sensing at least one audio signal including patient speech from a patient at a patient location during use of the communication system, the patient having a brain-related disorder and a prescribed treatment regimen for treating at least one aspect of the brain-related disorder, at least one first activity sensor for sensing at least one first activity signal indicative of a first activity of the patient, signal processing circuitry configured to process the at least one first activity signal and at least one second activity signal indicative of a second activity of the patient to generate at least one activity data signal, the activity data signal containing activity data indicative of whether the patient has complied with the treatment regimen, and at least one transmitting device at the patient location for transmitting the at least one activity data signal and at least one audio data signal based on the at least one audio signal to a receiving device at a monitoring location. In addition to the foregoing, other system aspects are described in the claims, drawings, and text forming a part of the disclosure set forth herein.

In an aspect, a method includes, but is not limited to, sensing with at least one audio sensor in a communication system at least one audio signal including patient speech from a patient at a patient location during use of the communication system, the patient having a brain-related disorder and a prescribed treatment regimen for treating at least one aspect of the brain-related disorder, sensing with at least one first activity sensor in the communication system at least one first activity signal indicative of a first activity of the patient, processing with signal processing circuitry the at least one first activity signal and at least one second activity signal indicative of a second activity of the patient to generate at least one activity data signal, the activity data signal containing data indicative of whether the patient has complied with the treatment regimen, and transmitting the at least one activity data signal and at least one audio data signal based on the at least one audio signal to a receiving device at a monitoring location with a transmitting device at the patient location. In addition to the foregoing, other method aspects are described in the claims, drawings, and text forming a part of the disclosure set forth herein.

In an aspect, a system includes, but is not limited to, a computing device and instructions that when executed on the computing device cause the computing device to control sensing with at least one audio sensor of at least one audio signal including patient speech from a patient at a patient location, the patient having a brain-related disorder and a prescribed treatment regimen for treating at least one aspect of the brain-related disorder, control sensing with at least one first activity sensor in an unobtrusive activity-detection system of at least one first activity signal indicative of a first activity of the patient, process with signal processing circuitry the at least one first activity signal and at least one second activity signal indicative of a second activity of the patient to generate at least one activity data signal, the activity data signal containing data indicative of whether the patient has complied with the treatment regimen, and control transmitting with a transmitting device at the patient location of the at least one activity data signal and at least one audio data signal based on the at least one audio signal to a receiving device at a monitoring location. In addition to the foregoing, other system aspects are described in the claims, drawings, and text forming a part of the disclosure set forth herein.

In an aspect, a computer program product includes, but is not limited to a non-transitory signal-bearing medium bearing one or more instructions for controlling sensing of at least one audio signal including patient speech from a patient at a patient location, the patient having a brain-related disorder and a prescribed treatment regimen for treating at least one aspect of the brain-related disorder, one or more instructions for controlling sensing with at least one first activity sensor in an unobtrusive activity-detection system of at least one first activity signal indicative of a first activity of the patient, one or more instructions for processing with signal processing circuitry the at least one first activity signal and at least one second activity signal indicative of a second activity of the patient to generate at least one activity data signal, the activity data signal containing data indicative of whether the patient has complied with the treatment regimen, and one or more instructions for controlling transmitting with a transmitting device at the patient location of the at least one activity data signal and at least one audio data signal based on the at least one audio signal to a receiving device at a monitoring location. In addition to the foregoing, other aspects of a computer program product including one or more non-transitory machine-readable data storage media bearing one or more instructions are described in the claims, drawings, and text forming a part of the disclosure set forth herein.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIG. 1 is a block diagram of a system for monitoring compliance of a patient with a treatment regimen.

FIG. 2 is a block diagram of an unobtrusive activity-detection system.

FIG. 3 is a block diagram showing further details of the unobtrusive activity-detection system of FIG. 2.

FIG. 4 is a block diagram of a monitoring system.

FIG. 5 illustrates an example embodiment of a thin computing device in which embodiments may be implemented.

FIG. 6 illustrates an example embodiment of a computing system in which embodiments may be implemented.

FIG. 7 is an illustration of an unobtrusive activity detection system implemented in a cell phone.

FIG. 8 is an illustration of an unobtrusive activity detection system implemented in a computing system.

FIG. 9 is an illustration of an unobtrusive activity detection system implemented in a microwave oven.

FIG. 10 is an illustration of an unobtrusive activity detection system implemented in a game system.

FIG. 11 is an illustration of an unobtrusive activity detection system implemented in a vehicle system.

FIG. 12 is an illustration of an unobtrusive activity detection system implemented in a kiosk.

FIG. 13 is an illustration of an unobtrusive activity detection system implemented in an intercommunication system.

FIG. 14 is an illustration of an unobtrusive activity detection system implemented in connection with a hair brush.

FIG. 15 is a flow diagram of a method relating to monitoring compliance of a patient with a prescribed treatment regimen.

FIG. 16 is a flow diagram of further aspects of the method of FIG. 15.

FIG. 17 is a flow diagram of further aspects of the method of FIG. 15.

FIG. 18 is a flow diagram of further aspects of the method of FIG. 15.

FIG. 19 is a flow diagram of further aspects of the method of FIG. 15.

FIG. 20 is a flow diagram of further aspects of the method of FIG. 15.

FIG. 21 is a flow diagram of further aspects of the method of FIG. 15.

FIG. 22 is a flow diagram of further aspects of the method of FIG. 15.

FIG. 23 is a flow diagram of further aspects of the method of FIG. 15.

FIG. 24 is a flow diagram of further aspects of the method of FIG. 15.

FIG. 25 is a flow diagram of further aspects of the method of FIG. 15.

FIG. 26 is a flow diagram of further aspects of the method of FIG. 15.

FIG. 27 is a flow diagram of further aspects of the method of FIG. 15.

FIG. 28 is a flow diagram of further aspects of the method of FIG. 15.

FIG. 29 is a block diagram of a computer program product including a signal-bearing medium.

FIG. 30 is a block diagram of a system including a computing device.

FIG. 31 is a flow diagram of a method of monitoring compliance of a patient with a prescribed treatment regimen.

FIG. 32 is a flow diagram of further aspects of the method of FIG. 31.

FIG. 33 is a flow diagram of further aspects of the method of FIG. 31.

FIG. 34 is a flow diagram of further aspects of the method of FIG. 31.

FIG. 35 is a flow diagram of further aspects of the method of FIG. 31.

FIG. 36 is a flow diagram of further aspects of the method of FIG. 31.

FIG. 37 is a flow diagram of further aspects of the method of FIG. 31.

FIG. 38 is a flow diagram of further aspects of the method of FIG. 31.

FIG. 39 is a flow diagram of further aspects of the method of FIG. 31.

FIG. 40 is a flow diagram of further aspects of the method of FIG. 31.

FIG. 41 is a flow diagram of further aspects of the method of FIG. 31.

FIG. 42 is a flow diagram of further aspects of the method of FIG. 31.

FIG. 43 is a flow diagram of further aspects of the method of FIG. 31.

FIG. 44 is a flow diagram of further aspects of the method of FIG. 31.

FIG. 45 is a flow diagram of further aspects of the method of FIG. 31.

FIG. 46 is a flow diagram of further aspects of the method of FIG. 31.

FIG. 47 is a flow diagram of further aspects of the method of FIG. 31.

FIG. 48 is a flow diagram of further aspects of the method of FIG. 31.

FIG. 49 is a block diagram of a computer program product including a signal-bearing medium.

FIG. 50 is a block diagram of a system including a computing device.

FIG. 51 is a block diagram system for monitoring compliance of a patient with a treatment regimen.

FIG. 52 is a flow diagram of a method is a flow diagram of a method of monitoring compliance of a patient with a prescribed treatment regimen.

FIG. 53 is a block diagram of a computer program product including a signal-bearing medium.

FIG. 54 is a block diagram of a system including a computing device.

FIG. 55 is a flow diagram of a method of monitoring compliance of a patient with a treatment regimen.

FIG. 56 is a block diagram of a computer program product including a signal-bearing medium.

FIG. 57 is a block diagram of a system including a computing device.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

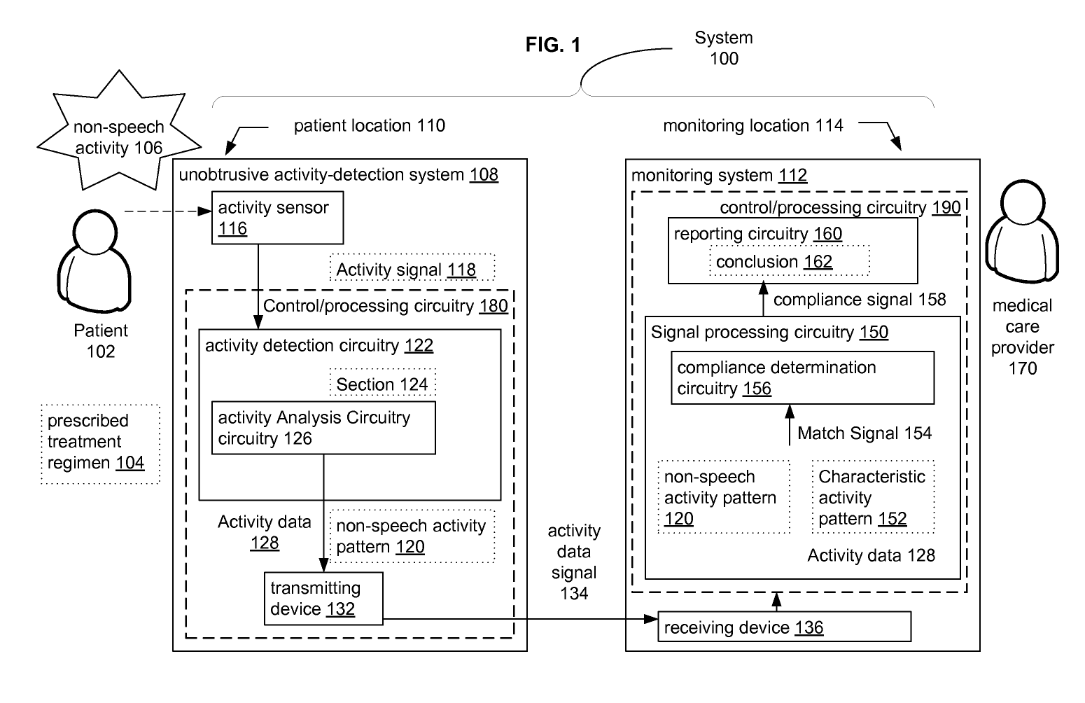

In an aspect, a patient 102 has a brain-related disorder, and treatment of the patient according to a prescribed treatment regimen 104 results in detectable changes in the patient's performance of one or more non-speech activities, relative to the patient's activity performance while in an untreated or partially treated state. In an aspect, failure of the patient to comply with a prescribed treatment regimen can be detected by monitoring the patient's activity-related activity patterns, and steps can be taken to address the patient's lack of compliance. FIG. 1 illustrates in block diagram form a system 100 for monitoring compliance of a patient 102 with a prescribed treatment regimen 104 based upon unobtrusive detection of a non-verbal activity of the patient, where the non-verbal activity corresponds to performance of non-speech activity 106 by the patient. System 100 includes unobtrusive activity-detection system 108 at patient location 110, which is used to detect non-verbal activity of the patient, and monitoring system 112 at monitoring location 114, which allows remote monitoring of patient compliance with prescribed treatment regimen 104 by a medical care provider 170 or other interested party or entity, e.g., a family member, an insurance company, etc.

In FIG. 1, and in other figures herein, in general, unless context dictates otherwise, solid lines are used to indicate standard components or steps, and dashed lines are used to represent optional components or steps. Unless context indicates otherwise, dotted lines are used to indicate data or information. Dashed lines may also be used to indicate signals.

System 100 monitors compliance of patient 102 with prescribed treatment regimen 104 by detecting and analyzing activity of patient 102 corresponding to performance of a non-speech activity 106.

Unobtrusive activity-detection system 108 includes at least one activity sensor 116 for sensing at least one activity signal 118 including a non-speech activity pattern 120 corresponding to performance of non-speech activity 106 by patient 102 at patient location 110. Unobtrusive activity-detection system 108 also includes activity detection circuitry 122, which is configured to identify at least one section 124 of the at least one activity signal 118 containing the non-speech activity pattern 120, and activity analysis circuitry 126 for processing the at least one section 124 of the at least one activity signal 118 to generate activity data 128 including data indicative of whether the patient has complied with the treatment regimen. In addition, unobtrusive activity-detection system 108 includes at least one transmitting device 132 for transmitting activity data signal 134 including activity data 128 including data indicative of whether the patient has complied with the treatment regimen. Transmitting device 132 transmits activity data signal 134 from patient location 110 to receiving device 136 at monitoring location 114.

Monitoring system 112 at monitoring location 114 includes at least one receiving device 136 for use at a monitoring location 114 for receiving an activity data signal 134 transmitted to the monitoring location 114 from patient location 110. Activity data signal 134 contains activity data 128 representing at least one non-speech activity pattern 120 in activity sensed from patient 102 with at least one activity sensor 116 in unobtrusive activity-detection system 108 at patient location 110 during performance of non-speech activity 106 by patient 102. Monitoring system 112 also includes signal processing circuitry 150, which is configured to analyze activity data signal 134 to determine whether activity data 128 represents at least one non-speech activity pattern 120 that matches at least one characteristic activity pattern 152. Signal processing circuitry 150 generates match signal 154 indicating a determination that non-speech activity pattern 120 matches a characteristic activity pattern 152. Monitoring system 112 also includes compliance determination circuitry 156, which is configured to determine whether patient 102 has complied with prescribed treatment regimen 104 based upon whether activity data 128 represents a non-speech activity pattern 120 that matches at least one characteristic activity pattern 152. Compliance determination circuitry 156 generates compliance signal 158. Monitoring system 112 also includes reporting circuitry 160, which is configured to report a conclusion 162 (regarding patient's compliance or lack thereof) based on the determination of whether the patient has complied with the prescribed treatment regimen 104, as indicated by compliance signal 158.

Both unobtrusive activity-detection system 108 and monitoring system 112 include control/processing circuitry, e.g., control/processing circuitry 180 in unobtrusive activity-detection system 108 and control/processing circuitry 190 in monitoring system 112, which includes the circuitry components specifically described herein and other circuitry components used to control operation of unobtrusive activity-detection system 108 and monitoring system 112, respectively.

In different embodiments, examples of which are described elsewhere here, different levels of signal processing take place in unobtrusive activity-detection system 108 at patient location 110 versus at monitoring system 112 at monitoring location 114. The location at which different signal processing aspects are performed may depend on availability of data storage space; speed, reliability and/or power consumption of data transmission between patient location 110 and monitoring location 114; and privacy concerns relating to storage and transmittal of patient data, among other considerations. As will be discussed in greater detail herein below, activity data signal 134 may contain raw activity data, information obtained from processed activity data, or both.

In an aspect, patient 102 has a brain-related disorder, and prescribed treatment regimen 104 is a treatment regimen prescribed to patient 102 for treating at least one aspect of the brain-related disorder. Brain-related disorders include, for example, mental disorders, psychological disorders, psychiatric disorders, traumatic disorders, lesion-related disorders, and/or neurological disorders, as discussed in greater detail elsewhere herein. Prescribed treatment regimen 104 may include a prescription for one or more therapeutic treatments, including medications, pharmaceuticals, nutraceuticals, therapeutic activities, diet, sleep, exercise, counseling, etc., to be used individually or in combination. In various aspects, prescribed treatment regimen 104 specifies type, quantity, and time course of any or all such therapeutic treatments.

Monitoring system 112 at monitoring location 114 allows medical care provider 170 or another interested individual or entity to remotely monitor compliance of patient 102 with prescribed treatment regimen 104. Monitoring location 114 may be, for example, a hospital, clinic, data center, or doctor's office. Monitoring location 114 may be a short distance away from patient location 110 (e.g., in another room of the same building, or even within the same room as patient location 110) or it may be in a separate building, a few miles away, or many miles away.

Systems as described herein can be used, for example, to monitor patient compliance with prescribed treatment regimen 104 at the request of or with the cooperation and/or authorization of patient 102, e.g., in the situation that the patient and/or the patient's caregiver wish to track the patient's compliance with the prescribed treatment regimen. In some cases, monitoring of patient compliance with a prescribed treatment regimen can be implemented at the request or requirement of a caregiver, insurance company, or other individual or entity, for example, as a condition of living in a group home, mental health care facility, or other institution, or as a condition of insurance reimbursement for treatment. In some cases, monitoring of compliance can be implemented without knowledge and/or authorization of the patient, e.g., in situations in which the patient is not capable of making decisions for his or her self or to fulfill a legal requirement.

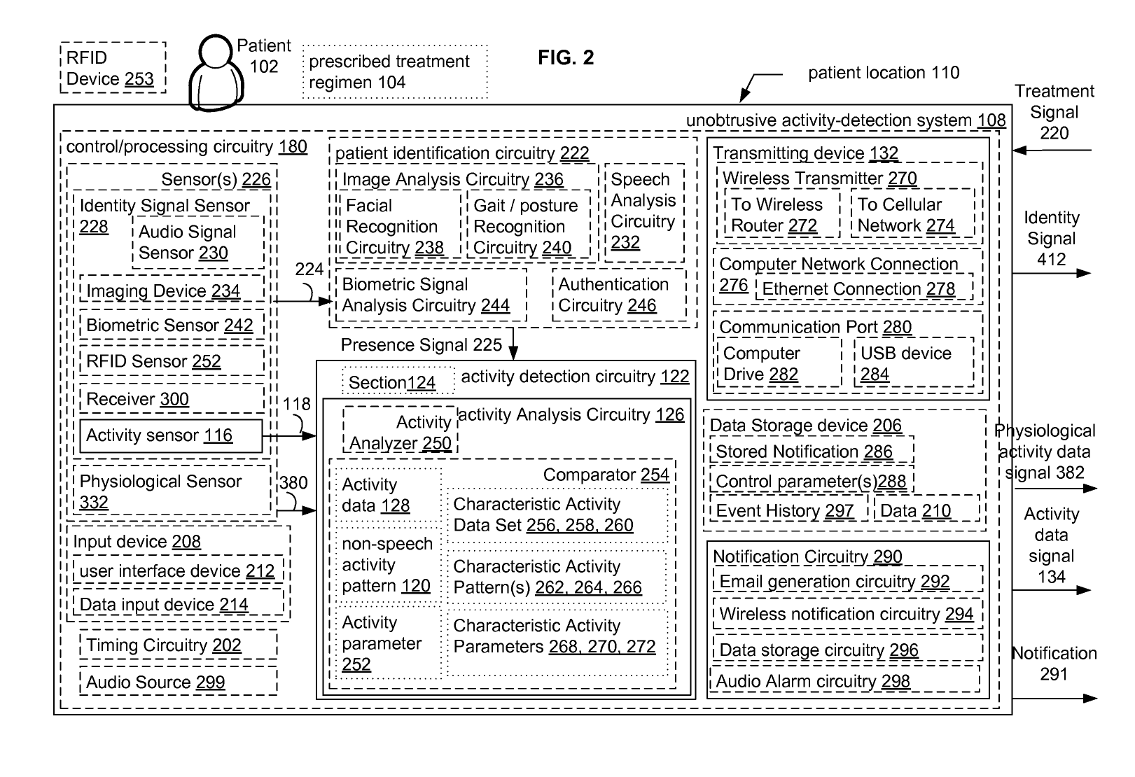

FIG. 2 illustrates components of unobtrusive activity-detection system 108 at patient location 110. As discussed above, unobtrusive activity-detection system 108 includes at least one activity sensor 116, activity detection circuitry 122, activity analysis circuitry 126, and at least one transmitting device 132. Activity detection circuitry 122, activity analysis circuitry 126, and other circuitry components as described herein include or form a part of control/processing circuitry 180.

Non-speech activity detected by unobtrusive activity-detection system 108 corresponds to one or more non-speech activity 106 performed by patient 102 (as shown in FIG. 1). For example, such activities may include various activities of daily life, or other activities or tasks performed routinely by patient 102, including, but not limited to, hygiene, washing, eating, dressing, brushing teeth, brushing hair, combing hair, preparing food, interacting with another person (e.g., in the same location or via an electronic device), interacting with an animal, interacting with a machine, interacting with an electronic device, or using an implement. In an aspect, such activities are performed by the patient 102 without prompting by unobtrusive activity-detection system 108. In an aspect, detection of non-speech activity-related activity is accomplished in a manner that is not noticeable to the patient, and does not interfere with the patient's daily routine. Unprompted activity refers to activity that is performed independent of any prompt by unobtrusive activity-detection system 108. Such activity can be considered “passively captured” in that capture of such activity is not predicated on the delivery of a prompt to the patient from unobtrusive activity-detection system 108. It should be noted, however, that, as used herein, unprompted activity in some cases includes activity produced by the patient in response to prompts or queries by another person, e.g., in the course of interaction with the person. In addition, activity produced by the patient that is not dependent on prior interaction with another person is also considered “spontaneous activity.”

Unobtrusive activity-detection system 108 may include various types of sensors 226, including various types of activity sensor(s) 116 for detecting activities that provide information regarding the patient's brain-related state. The patient's movements may be detected directly or indirectly with various types of sensors (including, but not limited to, pressure, force, capacitance, optical, motion, and acceleration sensors). Imaging sensors (e.g., cameras) can provide images of the patient that can be used to determine various aspects of motion of the patient. The patient's interaction with devices may be detected with user interface and input devices (e.g., keyboard, pointing device, or touchscreen) and/or device controls (including, but not limited to, controllers for game or entertainment devices or systems, appliances, vehicles, medical equipment, etc.). Interaction of the patient with other individuals, pets, or other animals, can be detected through image analysis, or through the use of proximity sensors to detect proximity of the patient to the individual or animal (with proximity assumed to correlate with interaction). Activity sensor 116 may be worn or carried by the patient, built into or attached to a device with which the patient interacts, or located in the patient's environment (e.g., a video camera in the patient's home).

In an aspect, activity detection circuitry 122 is configured to identity the at least one section 124 of the at least one activity signal containing non-speech activity pattern 120 from an activity signal 118 corresponding to unprompted performance of the non-speech activity by the patient.

In an aspect, unobtrusive activity-detection system 108 includes timing circuitry 202 configured to control timing of operation of at least a portion of unobtrusive activity-detection system 108 to perform substantially continuously sensing the at least one activity signal 118 with the at least one activity sensor 116. In an aspect, timing circuitry 202 includes a clock or timer device. For example, timing circuitry 202 may be configured to cause sensing to be performed substantially continuously by causing samples to be collected from the activity sensor 116 (e.g., via an A/D converter, not shown) at a fixed sampling rate that is sufficiently high to capture any meaningful variations in the activity sensed by the sensor (e.g., at least the Nyquist rate). The sampling rate may be determined by hardware or software, and may be factory pre-set or controllable by the user (e.g., the sampling rate may be determined by one or more control parameters 288 stored in data storage device 206, which may be set during manufacture of unobtrusive activity-detection system 108, or entered by a user of the system via input device 208.) For example, in an aspect, control/processing circuitry 180 includes an A/D converter, with the sampling rate of the A/D converter controlled by timing circuitry 202.

In another aspect, timing circuitry 202 is configured to control timing of operation of at least a portion of the system to perform intermittently at least one of sensing the at least one activity signal 118 with the at least one activity sensor 116, identifying the at least one section 124 of the at least one activity signal containing the non-speech activity pattern with the activity detection circuitry 122, processing the at least one section of the at least one activity signal to generate activity data 128 including data indicative of whether the patient has complied with the treatment regimen with the activity analysis circuitry 126, and transmitting an activity data signal 134 including the activity data 128 including data indicative of whether the patient has complied with the treatment regimen from the patient location 110 to a receiving device at a monitoring location with the at least one transmitting device 132. For example, in an aspect, intermittent sensing of the at least one activity signal 118 is controlled by using software to determine sampling rate and times at which sampling is performed, with appropriately selected control parameters 288 stored in data storage device 206. Alternatively, in an aspect, activity is sensed substantially continuously with activity sensor 116, but either activity detection circuitry 122 and/or activity analysis circuitry 126 is configured to process the activity signal 118 and/or section 124 intermittently rather than continuously. In another aspect, activity signal 118 is sampled on a substantially continuous basis, but transmitting device 132 is configured (with hardware or software) to transmit activity data signal 134 to the monitoring location only intermittently (once an hour, once a day, etc.). Intermittent performance of sampling, data transmission, and/or other system functions include performance at uniform intervals, any sort of non-uniform intermittent pattern (e.g., at a high frequency during some parts of the day and lower frequency during other parts of the day), or at random or quasi-random intervals (e.g., as determined by a random number generator). In an aspect, timing of system functions is controlled in part by timing circuitry 202 and in part in response to some other sensed parameter or other inputs; for example, a basic schedule may be determined by timing circuitry 202 but if it is determined that the subject is asleep or is not present, or if the data cannot be transmitted due to low signal strength, low battery power, etc., the scheduled function may be delayed until suitable conditions are obtained. Data storage device 206 is used to store data 210 that includes any or all of activity signal 118, section 124 of activity signal, and activity data 128, as such data are obtained. Data thus stored can be retrieved from data storage device 206 for transmission with transmitting device 132 intermittently. Data storage device 206 may be any of various types of data storage and/or memory devices.

In an aspect, timing circuitry 202 is configured to control timing of operation of at least a portion of the system to perform according to a schedule at least one of sensing the at least one activity signal with the at least one activity sensor 116, identifying the at least one section 124 of the at least one activity signal containing the non-speech activity pattern 120 with the activity detection circuitry 122, processing the at least one section 124 of the at least one activity signal to generate activity data 128 including data indicative of whether the patient has complied with the treatment regimen the activity analysis circuitry, and transmitting an activity data signal 134 including the activity data including data indicative of whether the patient has complied with the treatment regimen from the patient location to a receiving device at a monitoring location with the at least one transmitting device 132. Performance of the aforementioned steps according to a schedule can be controlled by timing circuitry 202 configured by hardware and software, using control parameters 288, including sampling rate and times at which sampling, processing of activity signal 118 and/or section 124, and transmission of activity data signal 134 are to be performed. The timing of these steps can be determined by control parameters 288, which may be set or selected by a user, or preset during manufacture of the device, as described above. Unobtrusive activity-detection system 108 may include one or more power sources (not shown), e.g., a battery, a plug for connecting to an electrical outlet or communication port, e.g., a USB port, or any of various other types of power sources.

As noted above, in an aspect, unobtrusive activity-detection system 108 includes an input device 208. In various aspects, input device 208 includes one or more of a user interface device 212, which may be any of various types of user interface devices, or data input device 214, which is a data input device adapted to receive data from a computing device or other electrical circuitry. Such data may be received by a wired connection or wireless connection. In an aspect, input device 208 is used for receiving a treatment signal 220 indicative of initiation of treatment of the patient according to the treatment regimen. In an aspect, treatment signal 220 is received from a user (either the patient or a caregiver of the patient) via a user interface device 212. In another aspect, treatment signal 220 is received via data input device 214.

In an aspect, unobtrusive activity-detection system 108 includes patient identification circuitry 222, which is configured to determine a presence of the patient from at least one identity signal 224 sensed at the patient location, and to generate presence signal 225 which is provided to activity detection circuitry 122. In an aspect, an identity signal 412 is transmitted from unobtrusive activity-detection system 108 to a monitoring system at the monitoring location. Identity signal 412 may be the same as identity signal 224, or may be a processed version of identity signal 224. In implementations in which unobtrusive activity-detection system 108 does not include patient identification circuitry 222, identity signal 412 may be transmitted to the monitoring location and processed by circuitry there to determine identity/presence of the patient. In implementations in which unobtrusive activity-detection system 108 include patient identification circuitry 222, identity signal 412 transmitted to the monitoring location so that the presence/identity of the patient may be determined from either the patient location or the monitoring location, or both, or the identity signal may be used for other purposes.

As noted previously, unobtrusive activity-detection system 108 includes activity sensor 116. In some aspects, activity signal 118 sensed by activity sensor 116 functions not only as a source of information regarding one or more activities performed by patient 102, but also as an identity signal 224 which is used to determine the identity of patient 102. In an aspect, patient identification circuitry 222 is configured to identify the at least one section 124 of the at least one activity signal containing the non-speech activity pattern based at least in part on a determination of the presence of the patient 102 by patient identification circuitry 222. In an aspect the at least one identity signal 224 includes at least a portion of the at least one activity signal 118, and patient identification circuitry 222 is configured to analyze the activity signal 118 to identify at least a portion of the at least one activity signal that resembles a known activity pattern of the patient. Accordingly, in this example activity sensor 116 is also identity signal sensor 228.

In order to use activity signal 118 as identity signal 224, it may be necessary to process activity signal 118 to determine the presence of the patient and simultaneously or subsequently process activity signal 118 with activity detection circuitry 122 to generate activity data 128. This can be accomplished by parallel processing of activity signal 118 by patient identification circuitry 222 and activity detection circuitry 122, or by processing activity signal 118 first with patient identification circuitry 222 and subsequently with activity detection circuitry 122. If the latter approach is used, generation of activity data signal 134 may not take place strictly in real time. Activity data signal 134 can be identified through the use of other types of identity signal, as well, as described herein below.

In some aspects, identity signal sensor 228 is distinct from activity sensor 116. In an aspect, unobtrusive activity-detection system 108 includes an audio signal sensor 230 for sensing an audio signal including speech from patient 102 at the patient location, and patient identification circuitry 222 includes speech analysis circuitry 232 for identifying at least a portion of the audio signal that resembles known speech of the patient. In an aspect, activity detection circuitry 122 is configured to identify the at least one section of the at least one activity signal 118 by activity in activity signal 118 that corresponds (e.g., spatially and/or temporally) to the presence of patient 102 detected by speech analysis circuitry 232. For example, a continuous speech system may be used for identifying the speaker, as described in Chandra, E. and Sunitha, C., “A Review on Speech and Speaker Authentication System using Voice Signal Feature Selection and Extraction,” IEEE International Advance Computing Conference, 2009. IACC 2009, Page(s): 1341-1346, 2009 (DOI: 10.1109/IADCC.2009.4809211), which is incorporated herein by reference. In an aspect, patient identification circuitry 222 is configured to analyze identity signal 224 to determine the presence of the patient based on frequency analysis of the audio identity signal. Magnitude or phase spectral analysis may be used, as described in McCowan, I.; Dean, D.; McLaren, M.; Vogt, R.; and Sridharan, S.; “The Delta-Phase Spectrum With Application to Voice Activity Detection and Speaker Recognition,” IEEE Transactions on Audio, Speech, and Language Processing, 2011, Volume: 19, Issue: 7, Page(s): 2026-2038 (DOI: 10.1109/TASL.2011.2109379), which is incorporated herein by reference.

In an aspect, unobtrusive activity-detection system 108 includes an imaging device 234 for sensing an image at the patient location, wherein the patient identification circuitry 222 includes image analysis circuitry 236 for identifying a presence of the patient in the image. For example, in an aspect image analysis circuitry 236 includes facial recognition circuitry 238, configured to analyze the image to determine the presence of the patient through facial recognition. For example, in an aspect facial recognition circuitry 238 uses approaches as described in Wheeler, Frederick W.; Weiss, R. L.; and Tu, Peter H., “Face Recognition at a Distance System for Surveillance Applications,” Fourth IEEE International Conference on Biometrics: Theory Applications and Systems (BTAS), 2010 Page(s): 1-8 (DOI: 10.1109/BTAS.2010.5634523), and Moi Hoon Yap; Ugail, H.; Zwiggelaar, R.; Rajoub, B.; Doherty, V.; Appleyard, S.; and Hurdy, G., “A Short Review of Methods for Face Detection and Multifractal Analysis,” International Conference on CyberWorlds, 2009. CW '09, Page(s): 231-236 (DOI: 10.1109/CW.2009.47), both of which are incorporated herein by reference.

In an aspect, image analysis circuitry 236 includes gait/posture recognition circuitry 240, which is configured to analyze the image to determine the presence of the patient through gait or posture recognition. Identification of the patient based on gait analysis can be performed, for example, by methods as described in U.S. Pat. No. 7,330,566, issued Feb. 12, 2008 to Cutler, and Gaba, I. and Kaur P., “Biometric Identification on The Basis of BPNN Classifier with Other Novel Techniques Used For Gait Analysis,” Intl. J. of Recent Technology and Engineering (IJRTE) ISSN: 2277-3878, Vol. 2, issue 4, Sep. 2013, pp. 137-142, both of which are incorporated herein by reference.

In an aspect, unobtrusive activity-detection system 108 includes a biometric sensor 242 for sensing a biometric signal from the patient, wherein the patient identification circuitry 222 includes biometric signal analysis circuitry 244 for analyzing the biometric signal to determine the presence of the patient. Biometric identification can include face and gait recognition, as described elsewhere herein, and recognition based on a variety of other physiological or behavioral characteristics, such as fingerprints, voice, iris, retina, hand geometry, handwriting, keystroke pattern, etc., e.g., as described in Kataria, A. N.; Adhyaru, D. M.; Sharma, A. K.; and Zaveri, T. H., “A Survey of Automated Biometric Authentication Techniques” Nirma University International Conference on Engineering (NUiCONE), 2013, Page(s): 1-6 (DOI: 10.1109/NUiCONE.2013.6780190), which is incorporated herein by reference. U.S. Pat. No. 8,229,178 issued Jul. 24, 2012 to Zhang et al., which is incorporated herein by reference, describes a method for acquiring a palm vein image with visible and infrared light and extracting features from the image for authentication of individual identity. Biometric identification can be based on imaging of the retina or iris, as described in U.S. Pat. No. 5,572,596 issued to Wildes et al. on Nov. 5, 1996 and U.S. Pat. No. 4,641,349 issued to Flom et al. on Feb. 3, 1987, each of which is incorporated herein by reference. Combinations of several types of identity signals can also be used (e.g., speech and video, as described in Aleksic, P. S. and Katsaggelos, A. K. “Audio-Visual Biometrics,” Proceedings of the IEEE Volume: 94, Issue: 11, Page(s): 2025-2044, 2006 (DOI: 10.1109/JPROC.2006.886017), which is incorporated herein by reference).

In an aspect, user interface device 212 is used for receiving an input indicative of at least one authentication factor from the user, and patient identification circuitry 222 includes authentication circuitry 246 for determining the presence of the patient based on the at least one authentication factor. The at least one authentication factor may include, for example, a security token, a password, a digital signature, and a cryptographic key. In an aspect, an authentication factor is received by unobtrusive activity-detection system via a user interface device 212. User interface device 212 can include various types of user interface devices or controls as are well known to those of ordinary skill in the art, including, but not limited to, keyboards, touchpads, touchscreens, pointing devices, (e.g., a mouse), joysticks, tracking balls, graphic interfaces, styluses, microphones or other voice interfaces, motion tracking interfaces, gesture interfaces (e.g., via a Kinect® or the like), brain-computer interfaces, buttons, or switches. User interface device 212 can be integral to a communication device, e.g., a key pad of a cell phone. One or more user interface device 212 in unobtrusive activity-detection system 108 can be used to receive various types of user interfaces relating to operation of unobtrusive activity-detection system 108, not limited to entry of an authentication factor. In an aspect, data input device 214 is used to receive a data signal, which is used as the identity signal, and patient identification circuitry 222 is configured to determine the presence of the patient based on the data signal.

In an aspect, unobtrusive activity-detection system 108 includes a receiver 300 for receiving a cell phone identification code, wherein the identity signal 224 is a cell phone identification code, and wherein the patient identification circuitry 222 is configured to determine the presence of the patient based on the cell phone identification code. The cell phone identification code may be, for example, an electronic serial number, a mobile identification number, and a system identification code.

In an aspect, unobtrusive activity-detection system 108 includes a radio frequency identification (RFID) sensor 252 for receiving an RFID signal from an RFID device 253 carried by or otherwise associated with patient 102, wherein the identity signal 224 is an RFID signal, and wherein the patient identification circuitry 222 is configured to determine the presence of the patient based on the RFID signal. In an aspect, RFID device 253 is a passive RFID in a tag or chip associated with the patient. In an aspect, RFID sensor 252 is an active RFID reader.

In an aspect, patient identification circuitry 222 is configured to distinguish the presence of patient 102 from the presence of another individual. In the event that the activity of another individual is detected by unobtrusive activity-detection system 108, activity detected from the other individual should not be used to determine the compliance of patient 102 with prescribed treatment regimen 104. Accordingly, in an aspect, patient identification circuitry 222 is configured to determine the presence of patient 102 by determining that information contained in the identity signal matches patient information associated with the patient. For some types of identity signal (e.g., a password or device identity code), an exact match can be obtained. In other cases, a match is obtained by using a windowing, thresholding, or distance measurement to determine whether the identity signal (or information contained there) matches sufficiently closely patient information associated with the patient. In an aspect, patient identification circuitry 222 is configured to distinguish the presence of the patient from the absence of the patient.

In an aspect, patient identification circuitry 222 generates presence signal 225 to indicate presence and/or identity of patient 102. In an aspect, presence signal 225 is provided as an input to activity detection circuitry 122. Presence of patient 102 may be indicated by a value of presence signal 225. For example, in some aspects, presence signal 225 is a binary signal; e.g., presence signal 225 has a high value if the patient is present or a low value if the patient is not present (or vice versa). In an aspect, activity data 128 is generated from activity signal 118 only when the value of presence signal 225 indicates that patient 102 is present. Alternatively, in some aspects presence signal 225 is a continuous valued signal that indicates the probability that the patient is present. For example, presence signal 225 has a value of 100 if there is 100 percent probability that the patient is present, a value of zero if there is zero percent probability that the patient is present, or an intermediate value if there is an intermediate probability that the patient is present. It will be appreciated that in some contexts, the determination of whether the patient is present or absent will be relatively straightforward, in which case a binary presence signal may be appropriate, whereas in others (e.g., in cases where the presence of the patient must be distinguished from the presence of other individuals, e.g., from a conference call) there is some likelihood of error in identifying the presence of the patient (with the likelihood of error potentially dependent upon the number and identity of the other individuals present), such that an indication of the probability that the patient is present may be more appropriate. In some aspects, various device functions (e.g., acquisition of activity data, performance of activity analysis, or transmission of activity data signal 134 to the monitoring location) are initiated in response to detection of the presence of patient 102. In some aspects, presence of patient 102 is a necessary but not sufficient condition for performance of particular device functions. For example, data may be collected at certain times of day, contingent upon the presence of patient 102. In another aspect, data is collected when patient 102 is present and initiates a particular activity.

In an aspect, activity detection circuitry 122 is configured to process the at least one activity signal to exclude at least one portion of the at least one activity signal that does not contain activity of patient 102, e.g., by excluding portions of the signal that contain no activity, or that contain activity of someone other than patient 102.

In an aspect, activity detection circuitry 122 is configured to identify at least one section 124 of the at least one activity signal containing an activity pattern corresponding to performance of an activity of daily life, for example, hygiene, washing, eating, dressing, brushing teeth, brushing hair, combing hair, preparing food, interacting with another person, interacting with an animal, interacting with a machine, interacting with an electronic device, or using an implement.

In an aspect, activity detection circuitry 122 is configured to identify at least one section of the at least one activity signal containing an activity pattern corresponding to performance of a motor activity. Examples of motor activities are typing, providing an input via an input device, providing an input via a keyboard, providing an input via a touchscreen, providing an input via a pointing device, controlling an entertainment device or system, controlling a game device or system, controlling a vehicle system, or walking.

In an aspect, unobtrusive activity-detection system 108 includes one or more physiological sensors 332. In some aspects, physiological sensor 332 provides physiological activity signal 380 to activity detection circuitry 122. In an aspect, information from physiological activity signal 380, taken in combination with activity signal 118, provides supplemental information that aids in determining compliance of patient 102 with prescribed treatment regimen 104. In some aspects, physiological activity data signal 382, including physiological activity data based on information from physiological activity signal 380 is transmitted to a monitoring system for further analysis.

In an aspect, activity analysis circuitry 126 is configured to process the at least one section 124 of the at least one activity signal to determine at least one non-speech activity pattern 120 of the patient. In an aspect, activity analysis circuitry 126 is configured to generate activity data 128 that includes the at least one non-speech activity pattern 120 of the patient. In addition, in an aspect, activity analysis circuitry 126 includes an activity analyzer 250 for assessing the at least one activity pattern to determine at least one activity parameter 252 indicative of whether the patient has complied with the treatment regimen, and wherein the activity analysis circuitry 126 is configured to generate activity data 128 that includes the at least one activity parameter. In various aspects, activity analysis circuitry 126 is configured to determine activity patterns or parameters. In an aspect, an activity pattern characterizes one or both of coarse and fine temporal patterns of activity (e.g., whether an activity occurs at a particular time of day, such as morning, afternoon, evening, or night; frequency of occurrence of the activity during a particular time window). In an aspect, an activity pattern characterizes amplitude or intensity of the activity (e.g., how forcefully the patient strikes a key on a keyboard, or magnitude of body movement). In an aspect, an activity pattern includes the location at which an activity is performed. In an aspect, an activity pattern includes details regarding the substance of the activity (e.g., if the activity is selecting a song on a music player, the activity pattern includes information regarding the specific song selected). Activity parameters may include, but are not limited to, activity performance error rate, activity performance rate, activity performance time, activity performance frequency (e.g., repetitions of an activity), activity performance variability (including amount of variability, or lack thereof), or activity performance accuracy.

In an aspect, activity analysis circuitry 126 includes a comparator 254 for comparing the at least one non-speech activity pattern 120 with at least one characteristic activity pattern 256 to determine whether the patient has complied with the treatment regimen. In an aspect, comparator 254 is configured to compare non-speech activity pattern 120 with a plurality of characteristic activity patterns 256, 258, and 260 (three characteristic activity patterns are provided as an example but the comparison is not limited to any specific number of characteristic activity patterns).

In an aspect, activity analysis circuitry 126 is configured to determine that the patient 102 has failed to comply with the treatment regimen. In an aspect, activity analysis circuitry 126 is configured to determine that the patient has complied with the treatment regimen.

In an aspect, activity analysis circuitry 126 is configured to determine whether the patient has complied with the treatment regimen based upon a determination of whether the activity data 128 represents at least one of a plurality of characteristic activity pattern(s) 262, 264, and 266. (Again, three patterns are provided as examples but comparison can be made to any number of characteristic activity patterns).

The result of the comparison performed by comparator 254 is a determination that the activity data 128 (or non-speech activity pattern 120 or activity parameter 252 derived therefrom) either does, or does not, match one or more characteristic activity data sets 256, 258, 260, patterns 262, 264, 266, or parameters 268, 270, 272. It will be appreciated that in various aspects, activity analysis circuitry 122 can be configured to determine both compliance and non-compliance, and additionally, or alternatively, level of compliance (either at specific levels or simply partial compliance). In an aspect, if there is a match, notification 291 is generated by notification circuitry 290 regarding whether the patient has complied with the prescribed treatment regimen. In practice, the comparison performed by comparator 254 (which may include thresholding, windowing, distance computation, for example, as discussed herein above) will result in production of a signal that indicates at least whether the patient has complied with the prescribed treatment regimen, and alternatively, or in addition, a level of compliance with the prescribed treatment regimen. In some cases, a medical care provider at the monitoring location (or another party or entity concerned with the patient's health and well-being, such as a parent, family member, caretaker, healthcare provider, insurance company, etc.) is notified only if the patient has failed to comply with the prescribed treatment regimen. Alternatively, in some aspects the medical care provider or other party/entity is notified when the patient is in compliance with the prescribed treatment regimen. In some aspects, notification can be provided by transmitting a notification 291 generated by notification circuitry 290 to the monitoring location with transmitting device 132, or to a wireless device, e.g., a remote device at the patient location, using wireless notification circuitry 294.

In an aspect, transmitting device 132 includes a wireless transmitter 270, which may, for example, transmit a signal to a wireless router 272 or a cellular network 274. In another aspect, transmitting device 132 includes a computer network connection 276, e.g., an Ethernet connection 278. In another aspect, transmitting device 132 includes a communication port 280. Communication port 280 may provide for communication with a computer drive 282 or USB device 284.

In an aspect, unobtrusive activity-detection system 108 includes notification circuitry 290 for generating a notification 291 indicative of whether the patient has complied with the treatment regimen. Notification circuitry 290 may include, for example, email generation circuitry 292 for generating an email notification, wireless notification circuitry 294 for generating a notification to be transmitted to a wireless device, data storage circuitry 296 for storing a notification in a data storage device, and audio alarm circuitry 298 for generating an audio notification to be delivered with audio source 299.

Compliance or lack thereof can be represented by appropriate text or numerical value in a displayed report or email, e.g., reported by notification circuitry 290, or represented by a binary value in data stored by data storage device 206. Alternatively, or in addition, level of compliance can be represented by a continuous value (e.g., percent compliance) or a text descriptor selected from a number of text descriptors corresponding to different levels of compliance (e.g., “non-compliance,” “low compliance,” “intermediate compliance,” “near-full compliance,” “full compliance”). In an aspect, notification circuitry 290 provides for formatting data included in notification 291 appropriately (e.g., by including appropriate text to accompany numerical data values) and for deciding whether and how to report the conclusion, based upon user preferences. For example, who is notified (patient versus medical care provider versus family member) or how notification is provided (stored in an event record, via email, or via a text message to a cell phone) may depend on the patient's level of compliance and the specifics of the patient. In some aspects, notification circuitry 290 can generate different levels of notifications depending on how serious a problem non-compliance is likely to be for the patient. Generating a notification may include retrieving a stored notification 286 from data storage device 206, e.g., selected from among one or more notifications 286 stored in data storage device 206. Notifications may take the form of text or numerical codes, for example.

In an aspect, notification circuitry 290 includes audio alarm circuitry 298 for generating an audio alarm, e.g., a tone or voice alert to be delivered via an audio source (e.g., a speaker, bell, buzzer, beeper, or the like). In an aspect, notification circuitry 290 provides a notification to patient 102, e.g., by generating an audio alarm via the audio source or causing a text message to be displayed on a display of unobtrusive activity-detection system 108, or a device in communication therewith, e.g., a cell phone or computing system used by patient 102. A notification to the patient could take the form of a reminder to take a medication or contact a medical care provider, for example. In another aspect, notification circuitry 290 uses wireless notification circuitry 294 to transmit a notification (e.g., via wireless transmitter 270) to a wireless device such as a pager, cell phone, or other wireless device used by a medical care provider or family member interested in tracking the status of the patient. In another aspect, notification circuitry 290 includes data storage circuitry 296 for storing a notification in a data storage device 206. For example, in an aspect, data storage device 206 provides for storage of a notification in event history 297 in conjunction with information regarding the time at which the notification was generated (obtained, for example from timing circuitry 202). In an aspect, information stored in event history 297 becomes a part of the subject's electronic medical records, and may ultimately be transferred to the monitoring system or other location. In an aspect, timing circuitry 202 includes a clock and/or timer, for example.

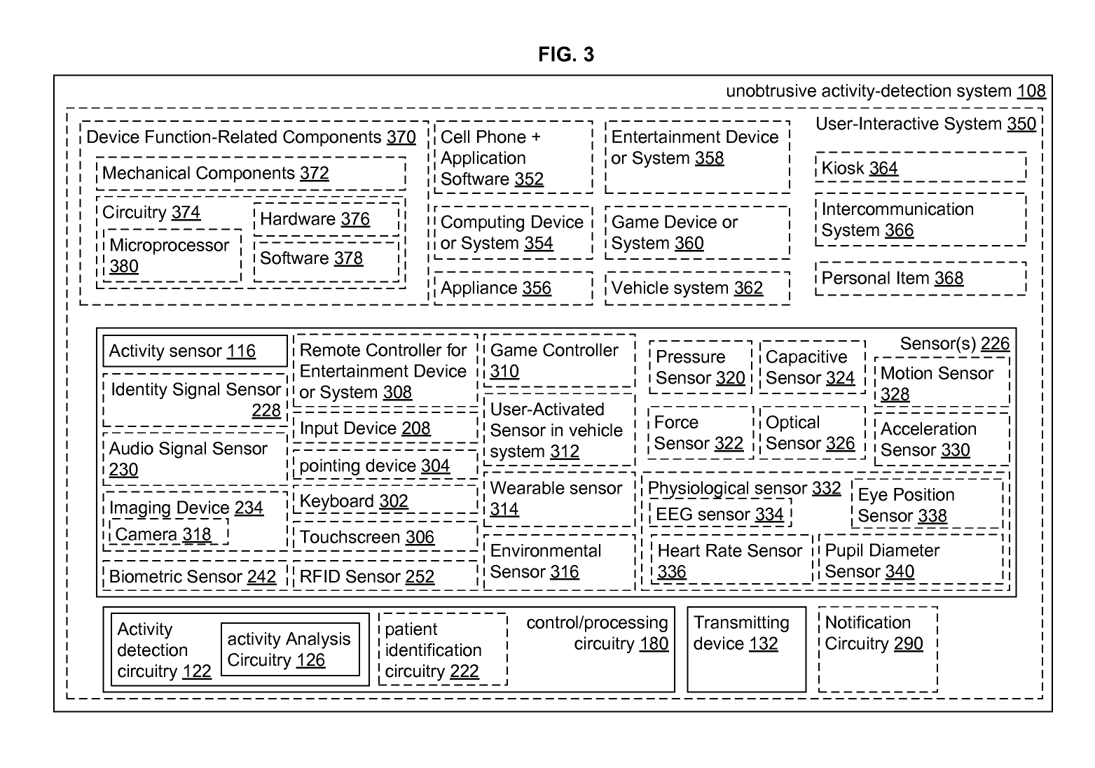

FIG. 3 depicts details of unobtrusive activity-detection system 108, showing additional details and additional and/or alternative components relative to what is shown in FIG. 2. As discussed in connection with FIG. 2, unobtrusive activity detection system 108 includes a variety of sensors 226, including one or more activity sensor 116 and one of more identity signal sensor 228. As discussed in connection with FIG. 2, in some aspects activity sensor 116 is the same as identity signal sensor 228, while in other aspects the activity and identity signal sensors are different sensors. Sensors 226 may include one or more identity signal sensor 228, including, but not limited to, one or more audio signal sensor 230, biometric sensor 242, RFID sensor 252, or imaging device 234. In an aspect, activity sensor 116 includes a camera 318 or other imaging device 234, which, in combination with appropriate hardware and software, may form a motion capture device (e.g., a Kinect®- or PlayStation® 4 Camera-type controller) that detects movements and/or gestures. In various aspects, such devices include depth sensing and IR reflectance technology, built-in color camera, infrared (IR) emitter, and microphone array.

A motion capture device can be used to detect activity of the subject during gaming or during daily living activities. In various aspects, camera 318 includes 2D and 3D cameras. Activity sensor 116 includes one or more devices of one or more types capable of sensing activity of the patient. In various aspects, the at least one activity sensor 116 includes one or more input device 208 (as described in connection with FIG. 2 which may be, for example, a keyboard 302, a pointing device 304 (e.g., a computer mouse), or a touchscreen 306. In various aspects, the at least one activity sensor 116 includes one or more remote controller for an entertainment device or system 308, or game controller 310. In various aspects, the at least one activity sensor includes a user-activated sensor in a vehicle system 312. In an aspect, activity sensor 116 is a wearable sensor 314 or an environmental sensor 316. In an aspect, an environmental sensor 316 includes one or more optical sensor 326 or camera 318 or other imaging device 234, in the environment of the subject. In an aspect, an environmental sensor includes a sensor in the environment of the subject that senses proximity of the patient to an object in the environment. In an aspect, an environmental sensor is a sensor attached to an animal or person in the environment. In an aspect, activity sensor 116 is attached to an item which the patient uses or interacts with, e.g., a comb, a toothbrush, an implement, a utensil, a tool, keys, etc. In an aspect, the at least one activity sensor 116 includes an imaging device 234, which may be, for example, a camera 318. In other aspect, activity sensor 116 includes one or more pressure sensor 320, force sensor 322, capacitive sensor 324, optical sensor 326, motion sensor 328, or acceleration sensor 330.

In an aspect, unobtrusive activity-detection system 108 includes at least one physiological sensor 332, operatively connected to the unobtrusive activity-detection system and configured to detect a physiological signal indicative of whether the patient has complied with the treatment regimen. For example, in an aspect, physiological sensor 332 includes an EEG sensor 334. In an aspect, EEG sensor 334 is configured to detect an event-related potential. Event-related potentials, or “ERPs” correspond to attention of a subject to an event (e.g., the event captures the subject's interest). ERPs normally occur at a fixed latency relative to the event of interest; thus, if the time of occurrence of the event of interest is known, ERGs can be detected based on their latency relative to the event of interest. In addition, it is also possible to detect ERPs in the EEG based on their characteristic shape, without information regarding when the event of interest occurred. Various ERP parameters, such as amplitude, latency, and/or topography are changed in patients with brain-related disorders. See, e.g., Hansenne, “Event-Related Brain Potentials in Psychopathology: Clinical and Cognitive Perspectives,” Psychologica Belgica 2006, vol. 46, iss. 1-2, pp. 5-36, and Wise et al., “Event-Related Potential and Autonomic Signs of Maladaptive Information Processing During an Auditory Oddball Task in Panic Disorder,” International Journal of Psychophysiology 74 (2009) 34-44, both of which are incorporated herein by reference. Moreover, in some cases treatment of brain-related disorder, e.g., with pharmaceuticals, at least partially restores the ERP parameters to values observed in individuals without the disorder, as described in Sumiyoshi et al., “Neural Basis for the Ability of Atypical Antipsychotic Drugs to Improve Cognition in Schizophrenia,” Frontiers in Behavioral Neuroscience,” 16 Oct. 2013, Volume 7, Article 140, which is incorporated herein by reference. In an aspect, the number and/or nature of ERPs detected in the patient's EEG provides additional or alternative information regarding compliance of the patient with the treatment regimen. In other aspects, physiological sensor 332 includes a heart rate sensor 336, an eye position sensor 338, or a pupil diameter sensor 340. Heart rate can be sensed by various approaches, using sensors in a fitness band (for example, of the type described in U.S. Pat. No. 9,113,795, which is incorporated herein by reference), sensors attached to the skin, etc. using various methods known in the art. Eye position can be sensed using a method and system as described in U.S. Pat. No. 8,808,195 to Tseng et al., which is incorporated herein by reference, or by other methods described herein or known to those skilled in the relevant art. Eye position may include static or fixed eye position/gaze direction or dynamic eye position/eye movement. Pupil diameter can be measured, for example, by methods as described in U.S. Pat. No. 6,162,186 to Scinto et al., which is incorporated herein by reference. Abnormal pupillary function is observed, for example, in patients with Alzheimer's disease (As discussed in Fotiou et al., “Pupil Reaction to Light in Alzheimer's disease: Evaluation of Pupil Size Changes and Mobility”, Aging Clin Exp Res 2007 October; 19(5):364-71 (Abstract), which is incorporated herein by reference.

Unobtrusive activity-detection system 108 can be constructed and implemented in a variety of embodiments in which different devices and/or device components provide the functionality described herein. In an aspect, unobtrusive activity-detection system 108 includes or is implemented on or in connection with various types of systems with which the patient interacts. In an aspect, unobtrusive activity-detection system 108 is built into such a user-interactive system 350. In another aspect, unobtrusive activity-detection system 108 is constructed separately but used in combination with such a user-interactive system 350. For example, unobtrusive activity-detection system 108 may be attached to user-interactive system 350, or operatively connected to user-interactive system 350. In various aspects, unobtrusive activity-detection system 108 can be constructed as a microprocessor-based system, either as a device that provides compliance monitoring in combination with some other functionality, or as a compliance monitoring system that is used independently, or as an add-on to a system which provides some other functionality.

In an aspect, activity sensor 116, activity detection circuitry 122, activity analysis circuitry 126, and transmitting device 132 are components of a cell phone 352 configured with application software. In another aspect, activity sensor 116, activity detection circuitry 122, activity analysis circuitry 126, and transmitting device 132 are components of a computing device or system 354. In another aspect, activity sensor 116, activity detection circuitry 122, activity analysis circuitry 126, and transmitting device 132 are components of an appliance 356 (e.g., a household appliance such as a microwave oven, a washing machine, or a coffee maker). In another aspect, activity sensor 116, activity detection circuitry 122, activity analysis circuitry 126, and transmitting device 132 are components of an entertainment device or system 358 (e.g., a TV, a DVD player, or a music player) or a game device or system 360. In yet another aspect, activity sensor 116, activity detection circuitry 122, activity analysis circuitry 126, and transmitting device 132 are components of a vehicle system 362. In an aspect, activity sensor 116, activity detection circuitry 122, activity analysis circuitry 126, and transmitting device 132 are components of a kiosk 364. In particular, kiosk 364 may be a medical kiosk used to provide health-related information, perform medical monitoring (e.g., take a blood pressure reading), dispense medication, and the like. In another example, kiosk 364 may be an entertainment or gaming kiosk, for example, located in a public venue such as a shopping mall or airport. In another aspect, activity sensor 116, activity detection circuitry 122, activity analysis circuitry 126, and transmitting device 132 are components of an intercommunication (“intercom”) system 366. In another aspect, activity sensor 116, activity detection circuitry 122, activity analysis circuitry 126, and transmitting device 132 are components of a personal item 368. For example, personal item 368 can be any of various types of personal items that are used by the patient in the course of carrying out activities of daily life, such that the patient's interaction with personal item 368 may indicate compliance of the patient with a prescribed treatment regimen. For example, personal item 368 may be a personal grooming article such as a comb, hair brush, or toothbrush; a tool or implement; a key or a key fob attached to one or more keys; a wearable item such as a wristwatch, an item of jewelry, eyeglasses, an article of clothing, footwear, hat, helmet, head covering, or hairband; or a wallet or purse. In an aspect, one or more of activity sensor 116, activity detection circuitry 122, activity analysis circuitry 126, and transmitting device 132 are operatively connected to personal item 368; e.g., one or more components may be packaged separately from personal item 368 but configured to be physically attached to personal item 368. In some aspects, one or more components of unobtrusive activity detection system 108 are not attached to the personal item 368, but communicate with at least one component attached to or built into personal item 368.

In addition to activity sensor 116, activity detection circuitry 122, activity analysis circuitry 126, and transmitting device 132 that form part of unobtrusive activity-detection system 108, user-interactive system 350 includes device function-related components 370, including, but not limited to, mechanical components 372 and/or circuitry 374, which may include hardware 376, software 378, and/or microprocessor 380.

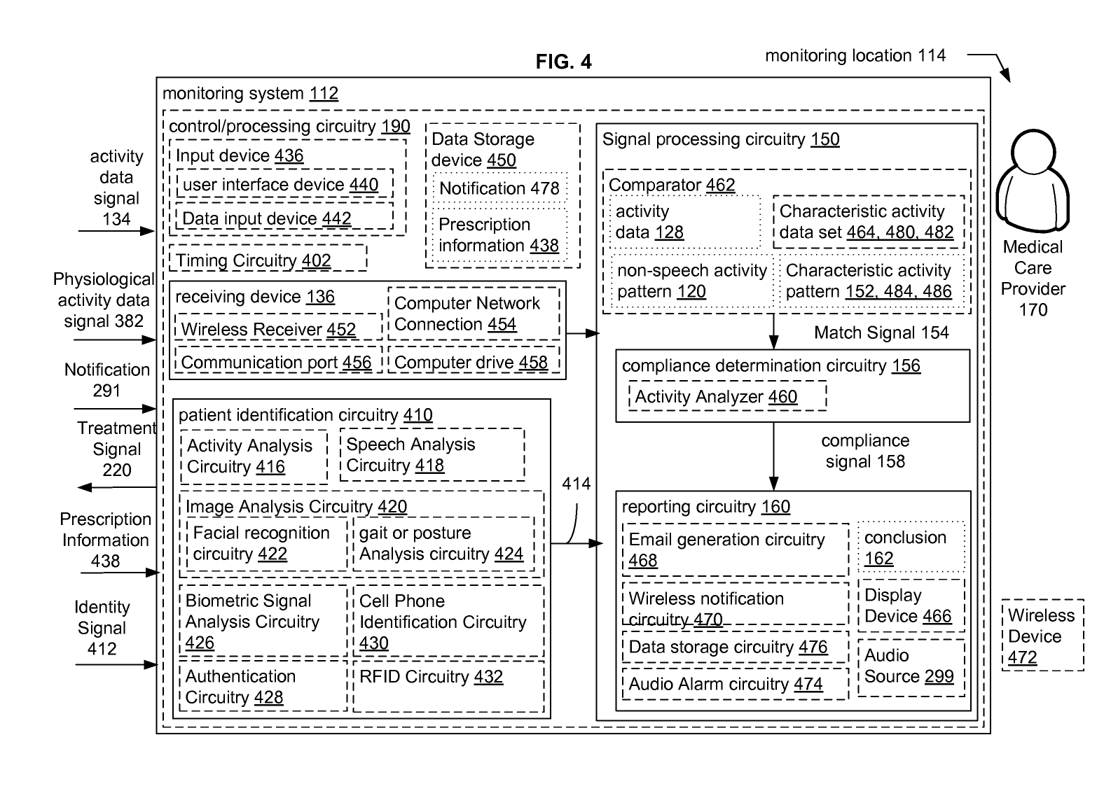

FIG. 4 depicts aspects of monitoring system 112. As described briefly in connection with FIG. 1, monitoring system 112 includes at least one receiving device 136 for use at a monitoring location 114 for receiving an activity data signal 134 transmitted to monitoring location 114 from a patient location. Activity data signal 134 contains activity data 128 representing at least one non-speech activity pattern 120 in activity sensed from a patient with at least one activity sensor in an unobtrusive activity-detection system (e.g., unobtrusive activity-detection system 108 at patient location 110 as shown in FIG. 1) during performance of the non-speech activity by the patient. Monitoring system 112 also includes signal processing circuitry 150, which is configured to analyze activity data signal 134 to determine whether the activity data 128 represents at least one non-speech activity pattern 120 that matches at least one characteristic activity pattern 152. In addition, monitoring system 112 includes compliance determination circuitry 156 configured to determine whether the patient has complied with the prescribed treatment regimen based upon whether the activity data 128 represents the non-speech activity pattern 120 that matches the at least one characteristic activity pattern 152, and reporting circuitry 160 configured to report a conclusion 162 based on the determination of whether the patient has complied with the treatment regimen.

In an aspect, signal processing circuitry 150 is configured to analyze the activity data signal 134 to identify at least one non-speech activity pattern that corresponds to unprompted performance of the non-speech activity by the patient. For example, in an aspect, signal processing circuitry 150 identifies non-speech activity based upon detectable patterns in the activity data signal, without relying upon information regarding timing of activity relative to a prompt. Analysis of activity data and/or activity patterns is performed substantially as discussed in connection with activity analysis circuitry 126 in FIG. 2.

In an aspect, monitoring system 112 includes timing circuitry 402, which may include a clock or timer device, and function in a manner substantially similar to timing circuitry 202 in unobtrusive activity-detection system 108 as described in connection with FIG. 2. In an aspect, timing circuitry 402 is configured to control timing of operation of at least a portion of the system to perform substantially continuously the operation of receiving the activity data signal 134 with the at least one receiving device 136. Receiving activity data signal 134 substantially continuously includes receiving a signal substantially without interruption, or sampling activity data signal 134 at a rate that is sufficiently high to capture any meaningful variations in the activity sensed by the sensor, as discussed herein above in connection with timing circuitry 202. In an aspect, timing circuitry 402 is configured to control timing of operation of at least a portion of monitoring system 112 to perform intermittently at least one of receiving the activity data signal 134 with the at least one receiving device 136, analyzing the activity data signal 134 with signal processing circuitry 150, determining with compliance determination circuitry 156 at monitoring location 114 whether the patient has complied with the treatment regimen, and reporting with reporting circuitry 160 a conclusion 162 based on the determination of whether the patient has complied with the prescribed treatment regimen.

In another aspect, timing circuitry 402 is configured to control timing of operation of at least a portion of the system to perform according to a schedule at least one of receiving the activity data signal 134 with the at least one receiving device 136, analyzing the activity data signal 134 with signal processing circuitry 150, determining with compliance determination circuitry 156 at the monitoring location 114 whether the patient has complied with the treatment regimen, and reporting with reporting circuitry 160 a conclusion 162 based on the determination of whether the patient has complied with the prescribed treatment regimen. Timing of operation of monitoring system 112 to form operations intermittently or according to a schedule can be controlled by timing circuitry 402 configured by hardware and software, using control parameters which may be set or selected by a user, or preset during manufacture of the device, as described above.

In some aspects, non-speech activity pattern 120 is an activity pattern corresponding to performance of a motor activity, which may include, for example, typing, providing an input via an input device, providing an input via a keyboard, providing an input via a touchscreen, providing an input via a pointing device, controlling a game device or system, controlling an entertainment device or system, controlling a vehicle system, or walking. In some aspects, non-speech activity pattern 120 is an activity pattern corresponding to performance of an activity of daily life, for example, hygiene, washing, eating, dressing, brushing teeth, brushing hair, combing hair, preparing food, interacting with another person, interacting with an animal, interacting with a machine, interacting with an electronic device, or using an implement.

In various aspects, activity data signal 134 contains activity data 128 including data from various types of sensors, as described in connection with FIG. 3, e.g., a pressure sensor, a force sensor, a capacitive sensor, an imaging device, a motion sensor, a motion capture device, an acceleration sensor, an optical sensor, a camera. In various aspects, activity data 128 represents one or more of a keystroke pattern, an activity performance pattern, an activity performance rate, an activity performance time, an activity performance frequency, an activity performance variability, an activity performance accuracy, or an activity performance error rate.

In an aspect, monitoring system 112 includes patient identification circuitry 410, which is configured to determine a presence of the patient from at least one identity signal 412 received by receiving device 136 at the monitoring location 114 from the patient location; in connection therewith signal processing circuitry 150 is configured to identify patient activity data corresponding to an activity of the patient based at least in part on the determination of the presence of the patient by the patient identification circuitry, as indicated by presence signal 414 generated by patient identification circuitry 410. In general, identity signals and determination of the presence of the patient are as described herein above in connection with FIG. 2.

In an aspect, identity signal 412 includes at least a portion of the activity data 128 in activity data signal 134, wherein patient identification circuitry 410 includes activity analysis circuitry 416 configured to analyze the activity data 128 to identify at least a portion of the activity data signal 134 containing activity data representing an activity pattern that matches a known activity pattern of the patient.

In an aspect, identity signal 412 includes a voice signal received from an audio sensor at the patient location, patient identification circuitry 410 includes speech analysis circuitry 418 for identifying at least a portion of the audio signal that resembles known speech of the patient, and signal processing circuitry 150 is configured to identify activity data corresponding to an activity of the patient by identifying activity data corresponding to a portion of the audio signal that resembles known speech of the patient.

In an aspect, identity signal 412 includes an image signal received from an imaging device at the patient location, wherein the patient identification circuitry includes image analysis circuitry 420 configured to analyze the image signal to determine the presence of the patient, and wherein the signal processing circuitry 150 is configured to identify activity data corresponding to an activity of the patient by identifying activity data corresponding to an image signal indicative of the presence of the patient. Image analysis circuitry 420 may include facial recognition circuitry 422 configured to analyze the image signal to determine the presence of the patient through facial recognition, or gait or posture analysis circuitry 424 configured to analyze the image signal to determine the presence of the patient through gait or posture recognition.

In another aspect, identity signal 412 includes a biometric signal from at least one biometric sensor at the patient location, and the patient identification circuitry 410 includes biometric analysis circuitry 426 configured to analyze the biometric signal to determine the presence of the patient, and signal processing circuitry 150 is configured to identify activity data corresponding to an activity of the patient by identifying activity data corresponding to a biometric signal indicative of a presence of the patient.

In another aspect, identity signal 412 include includes at least one authentication factor (e.g., one or more of a security token, a password, a digital signature, and a cryptographic key), and patient identification circuitry 410 includes authentication circuitry 428.

In another aspect, identity signal 412 includes a device identification code, which identifies unobtrusive activity-detection system 108, a component thereof, or an associated device. In an aspect, identity signal 412 includes a cell phone identification code (e.g., an electronic serial number, a mobile identification number, and a system identification code) and patient identification circuitry 410 includes cell phone identification circuitry 430. In some aspects, identity signal 412 includes a device identification code that identifies a computing system or device, a stand-alone microprocessor-based system, or a component thereof. A device identification code can serve to identify a patient (e.g., patient 102 in FIG. 1 and FIG. 2) providing the device thus identified is consistently used only by the patient. Identifying the patient based on device identification code may be done, for example, if some or all components of unobtrusive activity-detection system 108 are shared by multiple users but the device or component associated with the device identification code is used consistently by the patient. In an aspect, identity signal 412 includes an RFID signal, and patient identification circuitry 410 includes RFID circuitry 432.

In an aspect, monitoring system 112 includes input device 436, which is used, for example, for receiving prescription information 438 indicative of the treatment regimen prescribed to the patient. In an aspect, input device 436 includes a user interface device 440, for receiving information from a user (e.g., medical care provider 170). In another aspect, input device 436 includes a data input device 442, for receiving information from a computing device or other electrical circuitry (e.g., like data input device 214 described in connection with FIG. 2).

In an aspect, monitoring system 112 includes at least one data storage device 450, which may be used, for example, for storing prescription information 438 indicative of the treatment regimen prescribed to the patient.

In various aspects, receiving device 136 includes, for example, a wireless receiver 452, computer network connection 454, communication port 456, or computer drive 458.

In an aspect, compliance determination circuitry 156 includes an activity analyzer 460 for analyzing activity data 128 to determine the non-speech activity pattern 120, and a comparator 462 for comparing the non-speech activity pattern 120 represented by the activity data with the at least one characteristic activity pattern 152. In some aspects, comparator 462 is configured to compare the non-speech activity pattern 120 represented by activity data 128 with a plurality of characteristic activity patterns 152, 484, and 486 (three are depicted in FIG. 4, but comparison can be made with any number of characteristic activity patterns).

In another aspect, compliance determination circuitry 156 includes a comparator 462 for comparing the activity data 128 with at least one characteristic activity data set 464 representing at least one characteristic activity pattern 152. In an aspect, comparator 462 is configured to compare activity data 128 with a plurality of characteristic activity data sets 464, 480, and 482, each said characteristic activity data set representing a characteristic activity pattern (three are depicted in FIG. 4, but comparison can be made with any number of characteristic activity data sets). For example, in some aspects compliance determination circuitry 156 is configured to determine whether the patient has complied with the treatment regimen based upon a determination of whether the received activity data signal 134 represents at least one of a plurality of characteristic activity patterns 152.

In an aspect, compliance determination circuitry 156 is configured to determine that the patient has failed to comply with the treatment regimen. In another aspect, compliance determination circuitry 156 is configured to determine that the patient has complied with the treatment regimen.

In various aspects, reporting circuitry 160 includes a display device 466, email generation circuitry 468 for generating an email notification, wireless notification circuitry 470 for transmitting a notification to a wireless device 472 (which may be, for example, a cell phone used by medical care provider 170), audio alarm circuitry 474 for generating an audio alarm, or data storage circuitry 476 for storing a notification 478 in data storage device 450.

In an aspect, the at least one receiving device 136 is adapted to receive a physiological activity data signal 382 indicative of at least one physiological signal sensed with at least one physiological sensor operatively connected to the unobtrusive activity-detection system at the patient location. In an aspect, physiological activity data signal 382 is indicative of whether the patient has complied with the treatment regimen. In various aspects, physiological activity data signal 382 includes one or more of EEG data (including, for example, an event-related potential, wherein the event-related potential is related to performance of the non-speech activity by the subject), heart rate data, eye position data, or pupil diameter data.

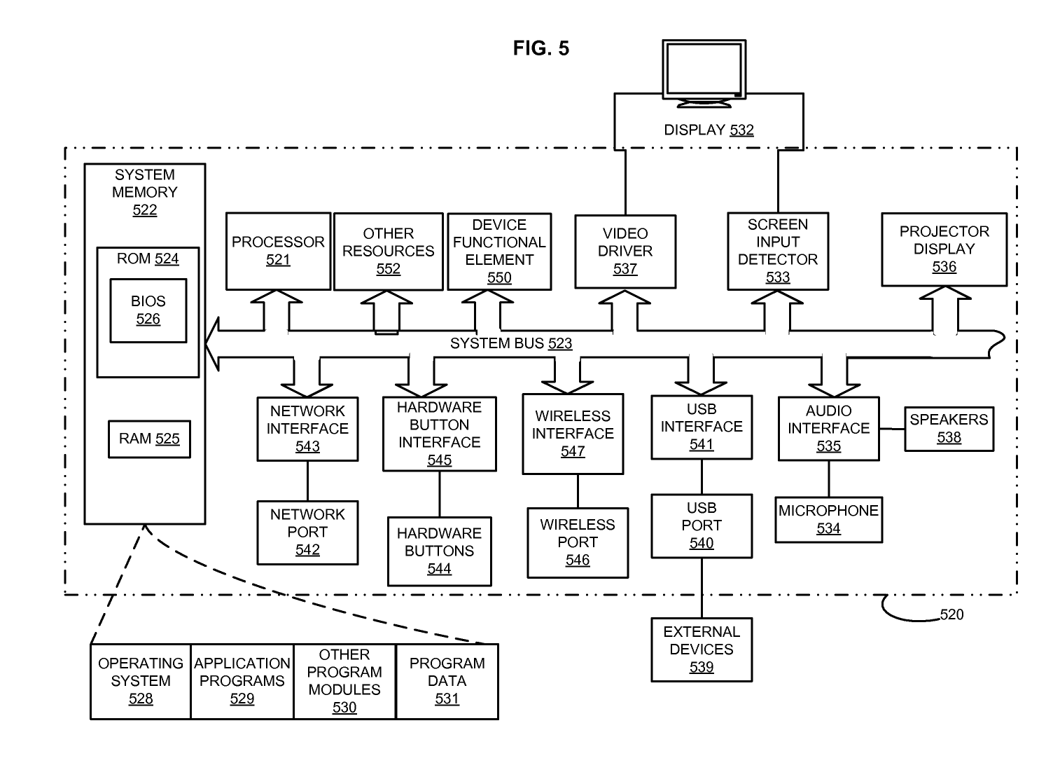

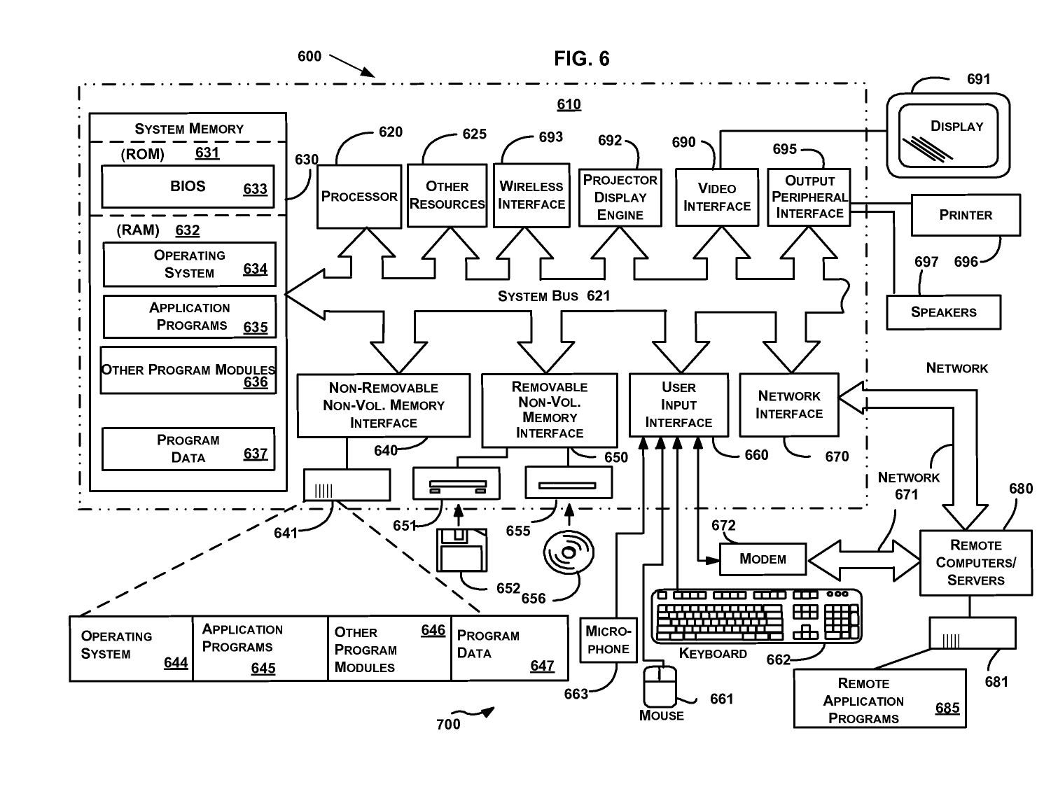

FIGS. 5 and 6 provide brief, general descriptions of environments in which embodiments may be implemented. FIG. 5 illustrates an example system that includes a thin computing device 520, which may be included in an electronic device that also includes one or more device functional element 550. For example, the electronic device may include any item having electrical or electronic components playing a role in a functionality of the item, such as a limited resource computing device, a wireless communication device, a mobile wireless communication device, an electronic pen, a handheld electronic writing device, a digital camera, a scanner, an ultrasound device, an x-ray machine, a non-invasive imaging device, a cell phone, a PDA, a Blackberry® device, a printer, a refrigerator, a car, and an airplane. In another example, the thin computing device may be included in a medical apparatus or device. In a further example, the thin computing device may be operable to communicate with a medical apparatus.

The thin computing device 520 includes a processor 521, a system memory 522, and a system bus 523 that couples various system components including the system memory 522 to the processor 521. The system bus 523 may be any of several types of bus structures including a memory bus or memory controller, a peripheral bus, and a local bus using any of a variety of bus architectures. In an aspect, the system memory includes read-only memory (ROM) 524 and random access memory (RAM) 525. A basic input/output system (BIOS) 526, containing the basic routines that help to transfer information between sub-components within the thin computing device 520, such as during start-up, is stored in the ROM 524. A number of program modules may be stored in the ROM 524 or RAM 525, including an operating system 528, one or more application programs 529, other program modules 530 and program data 531.

A user may enter commands and information into the computing device 520 through input devices, such as a number of switches and buttons, illustrated as hardware buttons 544, connected to the system via a suitable hardware button interface 545. Input devices may further include a touch-sensitive display with suitable input detection circuitry, illustrated as a display 532 and screen input detector 533. The output circuitry of the touch-sensitive display 532 is connected to the system bus 523 via a video driver 537. Other input devices may include a microphone 534 connected through a suitable audio interface 535, and a physical hardware keyboard (not shown). Output devices may include at least one display 532 and at least one speaker 538.

In addition to the display 532, the computing device 520 may include other peripheral output devices, such as a projector display 536. Other external devices 539 may be connected to the processor 521 through a USB port 540 and USB port interface 541, to the system bus 523. Alternatively, the other external devices 539 may be connected by other interfaces, such as a parallel port, game port or other port. External devices 539 include external input or output devices, e.g., a joystick, game pad, satellite dish, scanner, various types of sensors or actuators. Output signals include device control signals. The computing device 520 may further include or be capable of connecting to a flash card memory (not shown) through an appropriate connection port (not shown). The computing device 520 may further include or be capable of connecting with a network through a network port 542 and network interface 543, and through wireless port 546 and corresponding wireless interface 547 may be provided to facilitate communication with other peripheral devices, including other computers, printers, and so on (not shown). It will be appreciated that the various components and connections shown are examples and other components and means of establishing communication links may be used.

The computing device 520 may be primarily designed to include a user interface. The user interface may include a character, a key-based, or another user data input via the touch sensitive display 532. The user interface may include using a stylus (not shown). Moreover, the user interface is not limited to a touch-sensitive panel arranged for directly receiving input, but may alternatively or in addition respond to another input device such as the microphone 534. For example, spoken words may be received at the microphone 534 and recognized. Alternatively, the computing device 520 may be designed to include a user interface having a physical keyboard (not shown).

The device functional elements 550 are typically application specific and related to a function of the electronic device, and is coupled with the system bus 523 through an interface (not shown). The functional elements may typically perform a single well-defined activity with little or no user configuration or setup, such as a cell phone connecting with an appropriate tower and transceiving voice or data information, or communicating with an implantable medical apparatus, or a camera capturing and saving an image.

In certain instances, one or more elements of the thin computing device 520 may be deemed not necessary and omitted. In other instances, one or more other elements (e.g., other resources 552) may be deemed necessary and added to the thin computing device.

FIG. 6 illustrates an example embodiment of a computing system in which embodiments may be implemented, shown as a computing system environment 600. Components of the computing system environment 600 may include, but are not limited to, a computing device 610 having a processor 620, a system memory 630, and a system bus 621 that couples various system components including the system memory to the processor 620. The system bus 621 may be any of several types of bus structures including a memory bus or memory controller, a peripheral bus, and a local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus, also known as Mezzanine bus.

The computing system environment 600 typically includes a variety of computer-readable media products. Computer-readable media may include any media that can be accessed by the computing device 610 and include both volatile and non-volatile media, removable and non-removable media. By way of example, and not of limitation, computer-readable media may include computer storage media. By way of further example, and not of limitation, computer-readable media may include a communication media.

Computer storage media includes volatile and non-volatile, removable and non-removable media implemented in any method or technology for storage of information such as computer-readable instructions, data structures, program modules, or other data. Computer storage media includes, but is not limited to, random-access memory (RAM), read-only memory (ROM), electrically erasable programmable read-only memory (EEPROM), flash memory, or other memory technology, CD-ROM, digital versatile disks (DVD), or other optical disk storage, magnetic cassettes, magnetic tape, magnetic disk storage, or other magnetic storage devices, or any other medium which can be used to store the desired information and which can be accessed by the computing device 610. In a further embodiment, a computer storage media may include a group of computer storage media devices. In another embodiment, a computer storage media may include an information store. In another embodiment, an information store may include a quantum memory, a photonic quantum memory, or atomic quantum memory. Combinations of any of the above may also be included within the scope of computer-readable media.

Communication media may typically embody computer-readable instructions, data structures, program modules, or other data in a modulated data signal such as a carrier wave or other transport mechanism and include any information delivery media. The term “modulated data signal” means a signal that has one or more of its characteristics set or changed in such a manner as to encode information in the signal. By way of example, and not limitation, communication media include wired media, such as a wired network and a direct-wired connection, and wireless media such as acoustic, RF, optical, and infrared media.

The system memory 630 includes computer storage media in the form of volatile and non-volatile memory such as ROM 631 and RAM 632. A RAM may include at least one of a DRAM, an EDO DRAM, a SDRAM, a RDRAM, a VRAM, or a DDR DRAM. A basic input/output system (BIOS) 633, containing the basic routines that help to transfer information between elements within the computing device 610, such as during start-up, is typically stored in ROM 631. RAM 632 typically contains data and program modules that are immediately accessible to or presently being operated on by processor 620. By way of example, and not limitation, FIG. 6 illustrates an operating system 634, application programs 635, other program modules 636, and program data 637. Often, the operating system 634 offers services to applications programs 635 by way of one or more application programming interfaces (APIs) (not shown). Because the operating system 634 incorporates these services, developers of applications programs 635 need not redevelop code to use the services. Examples of APIs provided by operating systems such as Microsoft's “WINDOWS” are well known in the art.

The computing device 610 may also include other removable/non-removable, volatile/non-volatile computer storage media products. By way of example only, FIG. 6 illustrates a non-removable non-volatile memory interface (hard disk interface) 640 that reads from and writes for example to non-removable, non-volatile magnetic media. FIG. 6 also illustrates a removable non-volatile memory interface 650 that, for example, is coupled to a magnetic disk drive 651 that reads from and writes to a removable, non-volatile magnetic disk 652, or is coupled to an optical disk drive 655 that reads from and writes to a removable, non-volatile optical disk 656, such as a CD ROM. Other removable/nonremovable, volatile/non-volatile computer storage media that can be used in the example operating environment include, but are not limited to, magnetic tape cassettes, memory cards, flash memory cards, DVDs, digital video tape, solid state RAM, and solid state ROM. The hard disk drive 641 is typically connected to the system bus 621 through a non-removable memory interface, such as the interface 640, and magnetic disk drive 651 and optical disk drive 655 are typically connected to the system bus 621 by a removable non-volatile memory interface, such as interface 650.

The drives and their associated computer storage media discussed above and illustrated in FIG. 6 provide storage of computer-readable instructions, data structures, program modules, and other data for the computing device 610. In FIG. 6, for example, hard disk drive 641 is illustrated as storing an operating system 644, application programs 645, other program modules 646, and program data 647. Note that these components can either be the same as or different from the operating system 634, application programs 635, other program modules 636, and program data 637. The operating system 644, application programs 645, other program modules 646, and program data 647 are given different numbers here to illustrate that, at a minimum, they are different copies.

A user may enter commands and information into the computing device 610 through input devices such as a microphone 663, keyboard 62, and pointing device 661, commonly referred to as a mouse, trackball, or touch pad. Other input devices (not shown) may include at least one of a touch sensitive display, joystick, game pad, satellite dish, and scanner. These and other input devices are often connected to the processor 620 through a user interface 660 that is coupled to the system bus, but may be connected by other interface and bus structures, such as a parallel port, game port, or a universal serial bus (USB). Other devices that can be coupled to the system bus via other interface and bus structures include sensors of various types, for example.

A display 691, such as a monitor or other type of display device or surface may be connected to the system bus 621 via an interface, such as a video interface 690. A projector display engine 692 that includes a projecting element may be coupled to the system bus. In addition to the display, the computing device 610 may also include other peripheral output devices such as speakers 697 and printer 696, which may be connected through an output peripheral interface 695. Outputs may be sent to a variety of other types of devices, and are not limited to the example output devices identified here.

The computing system environment 600 may operate in a networked environment using logical connections to one or more remote computers, such as a remote computer 680. The remote computer 680 may be a personal computer, a server, a router, a network PC, a peer device, or other common network node, and typically includes many or all of the elements described above relative to the computing device 610, although only a memory storage device 681 has been illustrated in FIG. 6. The network logical connections depicted in FIG. 6 include a local area network (LAN) and a wide area network (WAN), and may also include other networks such as a personal area network (PAN) (not shown). Such networking environments are commonplace in offices, enterprise-wide computer networks, intranets, and the Internet.

When used in a networking environment, the computing system environment 600 is connected to the network 671 through a network interface, such as the network interface 670, the modem 672, or the wireless interface 693. The network may include a LAN network environment, or a WAN network environment, such as the Internet. In a networked environment, program modules depicted relative to the computing device 610, or portions thereof, may be stored in a remote memory storage device. By way of example, and not limitation, FIG. 6 illustrates remote application programs 685 as residing on computer medium 681. It will be appreciated that the network connections shown are examples and other means of establishing a communication link between the computers may be used.

In certain instances, one or more elements of the computing device 610 may be deemed not necessary and omitted. In other instances, one or more other elements (e.g., other resources 625) may be deemed necessary and added to the computing device.

FIGS. 5 and 6 illustrate generalized forms of circuitry-based systems, in which systems as depicted in FIGS. 1-4 may be implemented. Although specific embodiments are described herein, those skilled in the art will appreciate that methods and systems as described herein can be implemented in various ways. Reference is made herein to various circuitry systems/subsystems (e.g., patient identification circuitry 222, activity detection circuitry 122, notification circuitry 290 in FIG. 2, and patient identification circuitry 410, reporting circuitry 160, and signal processing circuitry 150 in FIG. 4) which may be considered to be control/processing circuitry, and/or components thereof. In general, control/processing circuitry (e.g., control/processing circuitry 180 and control/processing circuitry 190 in FIG. 1) includes any or all of digital and/or analog components, one or more processor (e.g., a microprocessor), and includes memory and additional components as described in connection with FIGS. 5 and 6.

In a general sense, those skilled in the art will recognize that the various embodiments described herein can be implemented, individually and/or collectively, by various types of electrical circuitry having a wide range of electrical components such as hardware, software, firmware, and/or virtually any combination thereof. Electrical circuitry includes electrical circuitry having at least one discrete electrical circuit, electrical circuitry having at least one integrated circuit, electrical circuitry having at least one application specific integrated circuit, electrical circuitry forming a computing device configured by a computer program (e.g., a computer configured by a computer program which at least partially carries out processes and/or devices described herein, or a microprocessor configured by a computer program which at least partially carries out processes and/or devices described herein), electrical circuitry forming a memory device, which may include various types of memory (e.g., random access, flash, read only, etc.), electrical circuitry forming a communications device (e.g., a modem, communications switch, optical-electrical equipment, etc.), and/or any non-electrical analog thereto, such as optical or other analogs (e.g., graphene based circuitry). In an embodiment, the system is integrated in such a manner that the system operates as a unique system configured specifically for the function of monitoring treatment compliance, and any associated computing devices of the system operate as specific use computers for purposes of the claimed system, and not general use computers. In an embodiment, at least one of the associated computing devices of the system is hardwired with a specific ROM to instruct the at least one computing device. In a general sense, those skilled in the art will recognize that the various aspects described herein which can be implemented, individually and/or collectively, by a wide range of hardware, software, firmware, and/or any combination thereof can be viewed as being composed of various types of “electrical circuitry.”

At least a portion of the devices and/or processes described herein can be integrated into a data processing system. A data processing system generally includes one or more of a system unit housing, a video display, memory such as volatile or non-volatile memory, processors such as microprocessors or digital signal processors, computational entities such as operating systems, drivers, graphical user interfaces, and applications programs, one or more interaction devices (e.g., a touch pad, a touch screen, an antenna, etc.), and/or control systems including feedback loops and control motors (e.g., feedback for sensing position and/or velocity; control motors for moving and/or adjusting components and/or quantities). A data processing system may be implemented utilizing suitable commercially available components, such as those typically found in data computing/communication and/or network computing/communication systems.

As discussed in connection with FIG. 1, transmitting device 132 in unobtrusive activity-detection system 108 and receiving device 136 in monitoring system 112 are configured to provide a communication link between the two locations. In various aspects, transmitting device 132 and receiving device 136 provide a wireless communication link. A wireless communication link may also be established between monitoring system 112 and wireless device 472, as shown in FIG. 4. In various aspects, a wireless communication link includes at least one of a radio frequency, wireless network, cellular network, satellite, WiFi, BlueTooth, Wide Area Network (WAN), Local Area Network (LAN), or Body Area Network (BAN) communication link. Various types of communication links are suitable for providing communication between two remote locations. Communication between locations remote from each other may take place over telecommunications networks, for example public or private Wide Area Network (WAN). In general, communication between remote locations is not considered to be suitably handled by technologies geared towards physically localized networks, e.g., Local Area Network (LAN) technologies operation at Layer 1/2 (such as the forms of Ethernet or WiFi). However, it will be appreciated that portions (but not the entirety) of communication networks used in remote communications may include technologies suitable for use in physically localized network, such as Ethernet or WiFi. In an aspect, system components are considered “remote” from each other if they are not within the same room, building, or campus. In an aspect, a remote system may include components separated by a few miles or more. Conversely, system components may be considered “local” to each other if they are located within the same room, building, or campus.

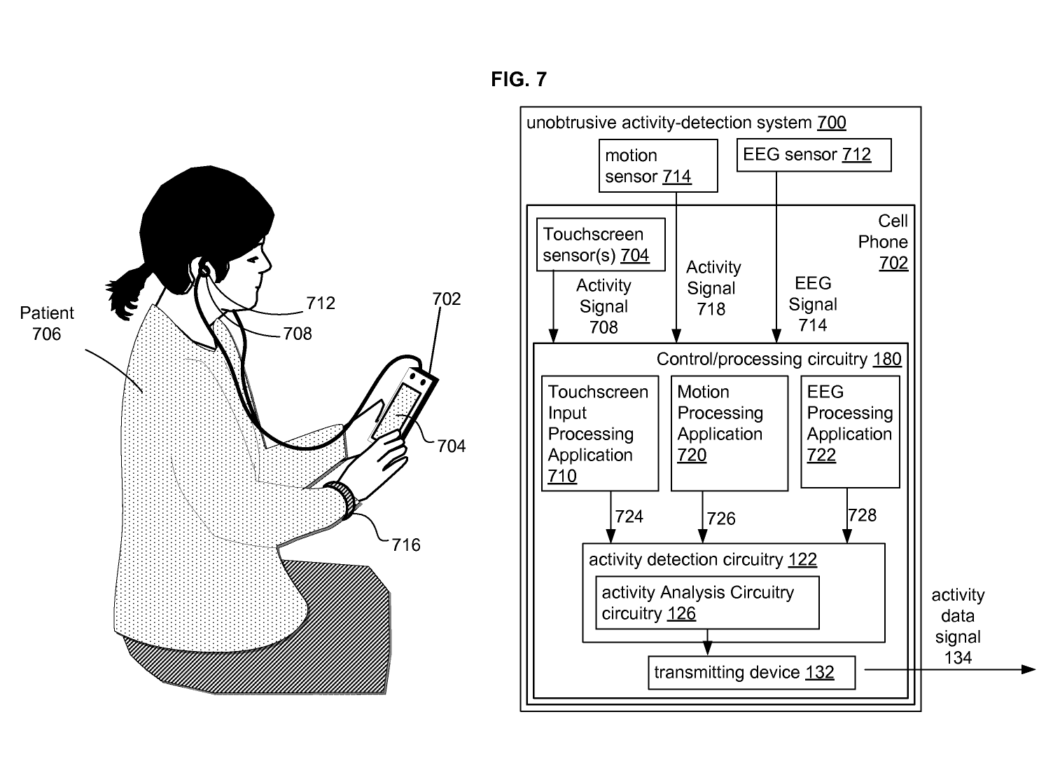

FIG. 7 illustrates an embodiment of an unobtrusive activity-detection system 700 that is based on a cell phone 702. In this example, activity detection circuitry 122, activity analysis circuitry 126, and transmitting device 132 are components of a cell phone 702, formed from standard cell phone hardware configured with application software. One or more touchscreen sensors 704, which are used for receiving instructions for controlling phone 702 entered by patient 706, serve as activity sensors. One or more activity signal 708 from touchscreen sensors 704 is processed by touchscreen input processing application 710. Activity signal 708 represents the motion of the patient's finger on the touchscreen, as sensed by touchscreen sensors 704. Touchscreen input processing application 710 determines the timing of entry of instructions by the patient. In an aspect, it is not necessary to determine the specific instructions entered by the patient, but only to determine how often the patient is using the phone, and/or how quickly the patient is entering instructions into the phone. However, in other aspects, the specific instructions can be detected, e.g., to determine whether the patient is choosing to listen to music, play a game, send or read email, receive a phone call, or place a phone call. An EEG (electroencephalogram) sensor 712 serves as a physiological sensor for providing further information relating to the brain-related functioning of patient 706. EEG sensor 712 includes electrodes built into earbuds (which are used by the patient 706 for listening to phone calls, music, or other audio outputs provided by cell phone 702). Sensed EEG signal 714 is processed by EEG processing application 722. Sensing of EEG signals with sensors that fit into the ear canal is described, for example, in U.S. Patent Publication 2003/0195588 to Fischell et al., and U.S. Patent Publication 2006/0094974 to Cain, both of which are incorporated herein by reference. See also Bleichner, et al., “Exploring Miniaturized EEG Electrodes for Brain-Computer Interfaces. An EEG You Do Not See?” Physiological Reports 2015, Vol. 3, Iss. 4, e12362, doi:10.14814/phy2.12363, which is incorporated herein by reference. In an aspect, EEG sensor 712 is used for detecting event-related potentials (ERPs) associated with a detectable event associated with operation of cell phone 702. In an aspect, the detectable event is an event that can be detected by control/processing circuitry 180 in cell phone 702. For example, in various aspects, the detectable event includes providing notification of the arrival of an incoming call to patient 706 (e.g., by ringing or vibration of cell phone 702), providing notification of the arrival of an email message or impending calendared event with an audible tone or a pop-up message. As used herein, a “detectable” event is an event that results in a detectable change in control/processing circuitry 180 of cell phone 702. In principle, the “detectable” event is also expected to be detectable by patient 706, at least at a sub-conscious level, with such detection of the event by the patient resulting in generation of an event-related potential that can be sensed with EEG sensor 712. Because changes in amplitude, latency, and/or topography of event-related potentials have been observed in subjects with various brain-related disorders (Hansenne, “Event-Related Brain Potentials in Psychopathology: Clinical and Cognitive Perspectives,” Psychologica Belgica 2006, vol. 46, iss. 1-2, pp. 5-36, which is incorporated herein by reference), changes in event-related potential production in response to a detectable event, or absence of an event-related potential in response to a detectable event provide information regarding the mental function of the patient, and hence whether the patient has complied with a prescribed treatment regimen. Motion sensor 714 in wristband 716 generates second activity signal 718 representing motion of patient 706. Second activity signal 718 is processed by motion processing application 720. Activity detection circuitry 122 receives signals 724, 726, and 728 from touchscreen input processing application 710, motion processing application 720, and EEG processing application 722, respectively, which are received by activity detection circuitry 122 and processed to generate activity data signal 134. Signal 724 from touchscreen input processing application 710 supplies to activity detection circuitry information regarding how often the patient 706 uses phone 702 (summarizing the patient's entry of instructions by category, e.g., by providing the number of times the person placed a phone call, the number of times the patient looked at email, and the number of hours per day spent listening to music). Signal 726 from motion processing application 720 provides information regarding the patient's activity level (sensed by motion sensor 714 in wristband 716), and signal 728 from EEG processing application 722 provides information regarding how attentive the patient is to a the detectable event (e.g., percent of the time that an ERP was produced in response to a notification regarding the arrival of an email). ERP information and activity patterns relating to patient motion and touchscreen activity are processed in combination to determine compliance of patient 706 with a prescribed treatment regimen.

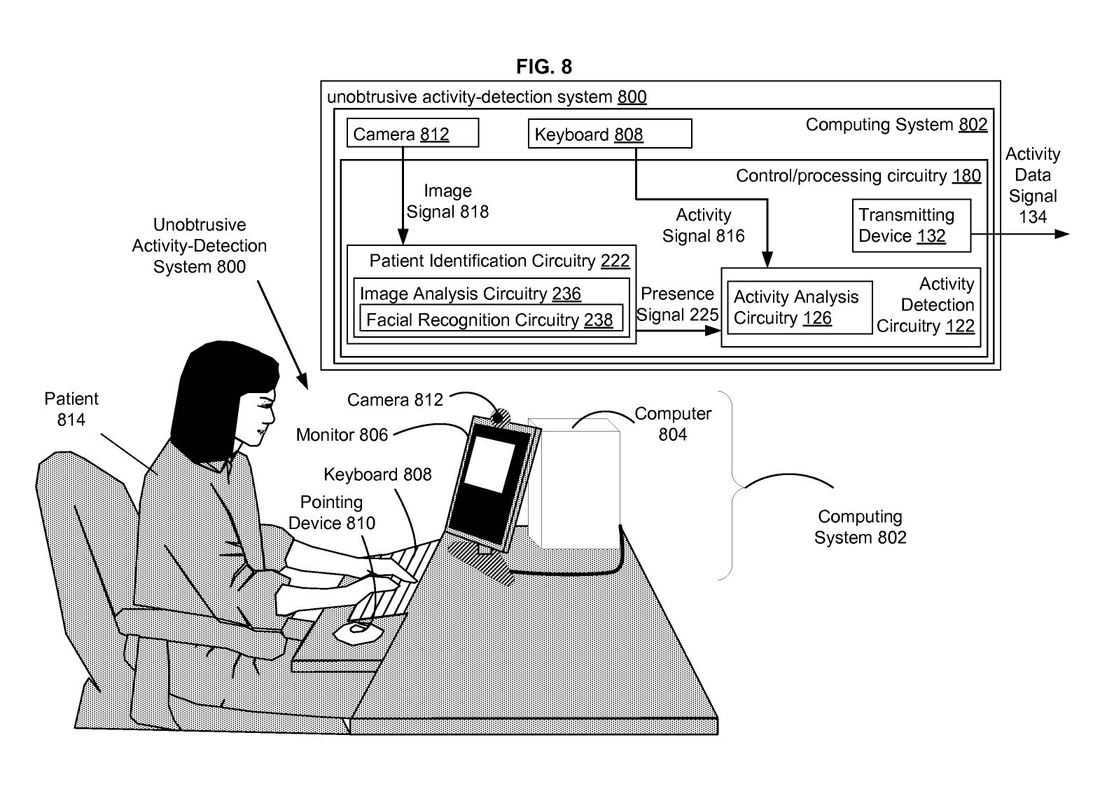

FIG. 8 depicts an embodiment of an unobtrusive activity-detection system 800, implemented in a computing system 802. Computing system 802 includes computer 804, monitor 806, keyboard 808, pointing device 810, and camera 812, which is built into monitor 806 in the present example. Computing system 802 is used by patient 814 to perform personal or work-related activities, such as (for example, and without limitation) creating and editing documents using word-processing software. In this example, keyboard 808 serves as an activity sensor, providing activity signal 816 to activity detection circuitry 122. Other components of unobtrusive activity-detection monitoring system 800 (e.g., activity analysis circuitry 126, and transmitting device 132) are components of a computing system 802. In addition, camera 812 provides an identify signal (image signal 818) to patient identification circuitry 222, where it is processed by facial recognition circuitry 238 in image analysis circuitry 236 to determine the identity/presence of patient 814 to generate presence signal 225. It will be appreciated that it may also be possible to determine the identity/presence of patient 814 by utilizing login/password information provided when patient 814 logs onto computer 804 (or logs into a specific piece of program or online accounts) for authentication. Activity signal 816 contains information regarding the patient's typing pattern, which is analyzed by activity analysis circuitry 126, to generate activity data signal 134, which is transmitted to a monitoring location by transmitting device 132. Activity analysis circuitry 126 may analyze typing patterns using, for example, techniques as described in U.S. Pat. No. 6,231,344 to Merzenich et al., U.S. Published Patent Application 2005/0084832 to Janssen et al., each of which is incorporated herein by reference

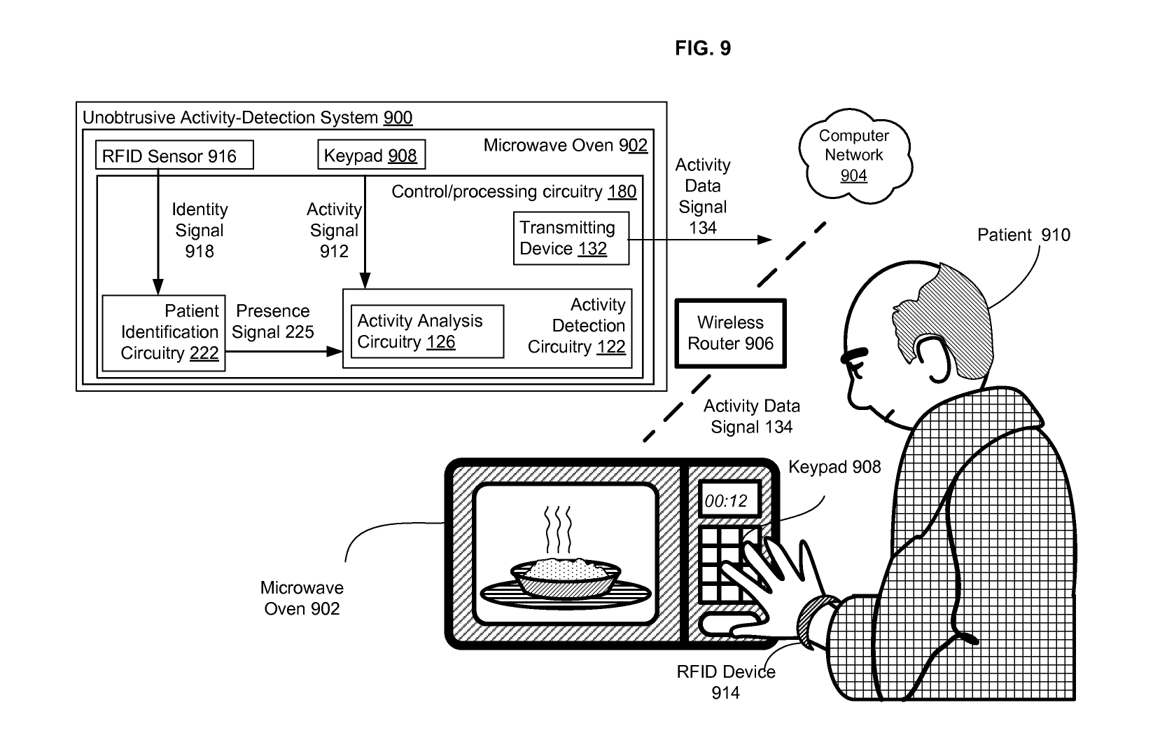

FIG. 9 depicts an embodiment of an unobtrusive activity-detection system 900 that is implemented in connection with a microwave oven 902. Microwave oven 902 is a “smart” oven that includes a circuitry that allows it to send data to and receive data from a computing network, for example, as described in, e.g., U.S. Pat. No. 8,631,063 to Helal et al., U.S. Pat. No. 9,113,795 to Hong et al., U.S. Pat. No. 8,667,112 to Roth et al., each of which is incorporated herein by reference. Microwave oven 902 includes control/processing circuitry 180 and communication circuitry (including transmitting device 132), allowing it to connect to the computer network 904 via a wireless router 906 or other wireless communication device (e.g., a cell phone or laptop computer). Activity detection circuitry 122, activity analysis circuitry 126, and transmitting device 132 are components of microwave oven 902. Keypad 908 of microwave oven 902 is used as an activity sensor, providing an activity signal 912 to activity detection circuitry 122. When patient 910 uses keypad 908 to operate microwave oven 902, activity signal 912 is sent to activity detection circuitry 122. In an aspect, the pattern of use of microwave oven, as indicated by activation of keypad 908 (e.g., time of day that it is used, frequency of use during the day) may be indicative of the brain-related functioning of the patient. For example, a depressed patient may be less likely to make the effort to prepare food, and will use the microwave oven less than usual. In other cases the patient may use the microwave more often than is typical for that patient, or at unusual times of the day or night. A patient that is showing symptoms of dementia may have difficulty pressing the keys on the keypad in the appropriate sequence in order to heat food. Accordingly, accuracy of operation of the microwave oven (e.g., whether the patient presses keys in the proper sequence to select cooking time and temperature and turns on the oven, and how many attempts it takes to operate the oven properly) may be indicative of the patient's alertness or coordination. Identity of patient 910 is determined by sensing an RFID signal from RFID device 914, using RFID sensor 916. Identity signal 918 from RFID sensor 916 is provided to patient identity circuitry 222, which generates presence signal 225, as discussed herein above. It is contemplated that RFID device 914 is a passive RFID device, but in other embodiments an active RFID could be used. RFID device 914 is depicted as taking the form of a wristband worn by patient 910, but it could be embodied in a necklace, a key fob, an implant, clothing, or other form. As an alternative, patient 910 could be identified by sensing an identification signal from a cell phone or smart watch carried by patient 910.

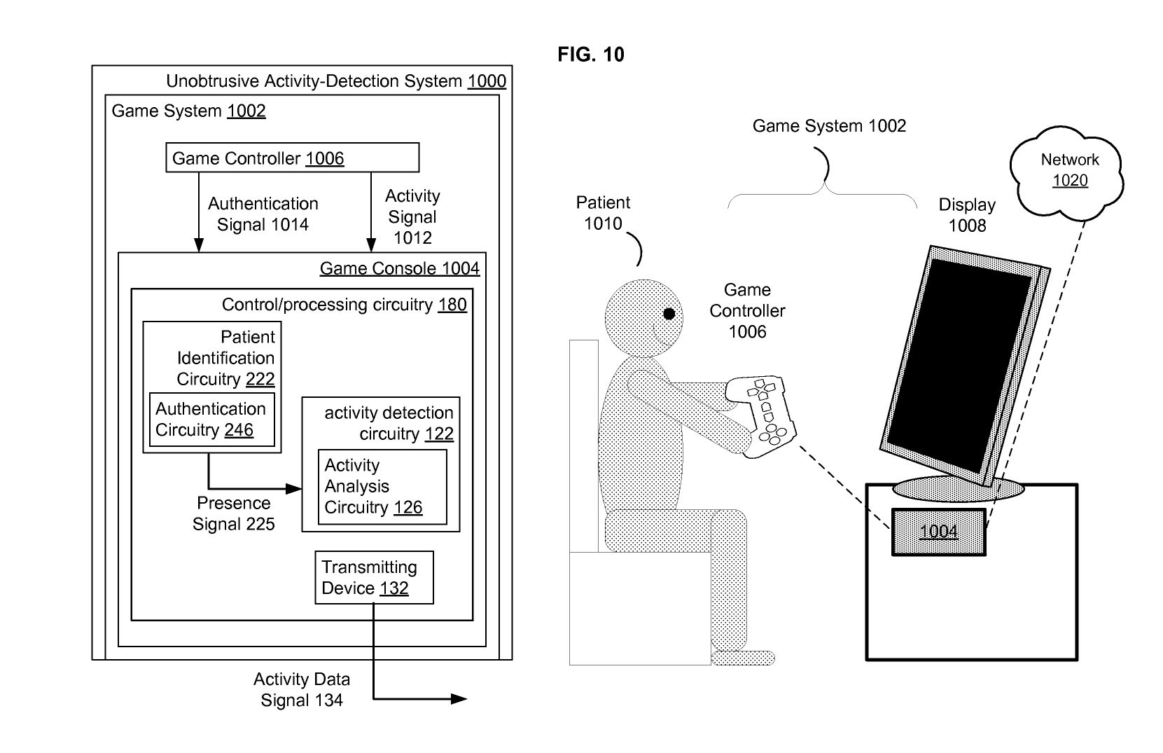

FIG. 10 depicts an example of an unobtrusive activity-detection system 1000 that is incorporated into a game system 1002. Game system 1002 includes a game console 1004, game controller 1006 for providing control signals to game console 1004, and display 1008 driven by video output from game console 1004. Game controller 1006 functions as an activity sensor; as patient 1010 plays the game, signals from game controller 1006 are used as activity signal 1012, which is processed by activity detection circuitry 122 and activity analysis circuitry 126 in game console 1004. Sensing and processing of game controller signals, e.g., to determine reaction times, may be substantially as described in U.S. Pat. No. 5,913,310 to Brown, or U.S. Pat. No. 6,186,145 to Brown, both of which are incorporated herein by reference. It will be appreciated that while Brown describes a video game designed primarily for health care-related teaching purposes, the video game may be for entertainment purposes, and need not include an educational or medical component. Activity detection circuitry 122 and activity analysis circuitry 126 include special-purpose hardware and/or software incorporated into game console 1004 (in the form of an add-on card or software). Username/password information entered into game controller 1006 by patient 1010 is used as an authentication signal 1014 processed by authentication circuitry 246 in patient identification circuitry 222 to generate presence signal 225 that indicates presence of the patient. Game console 1004 also includes transmitting device 132, which is used for communicating with network 1020, including transmitting activity data signal 134 to a monitoring location for processing as described elsewhere herein.

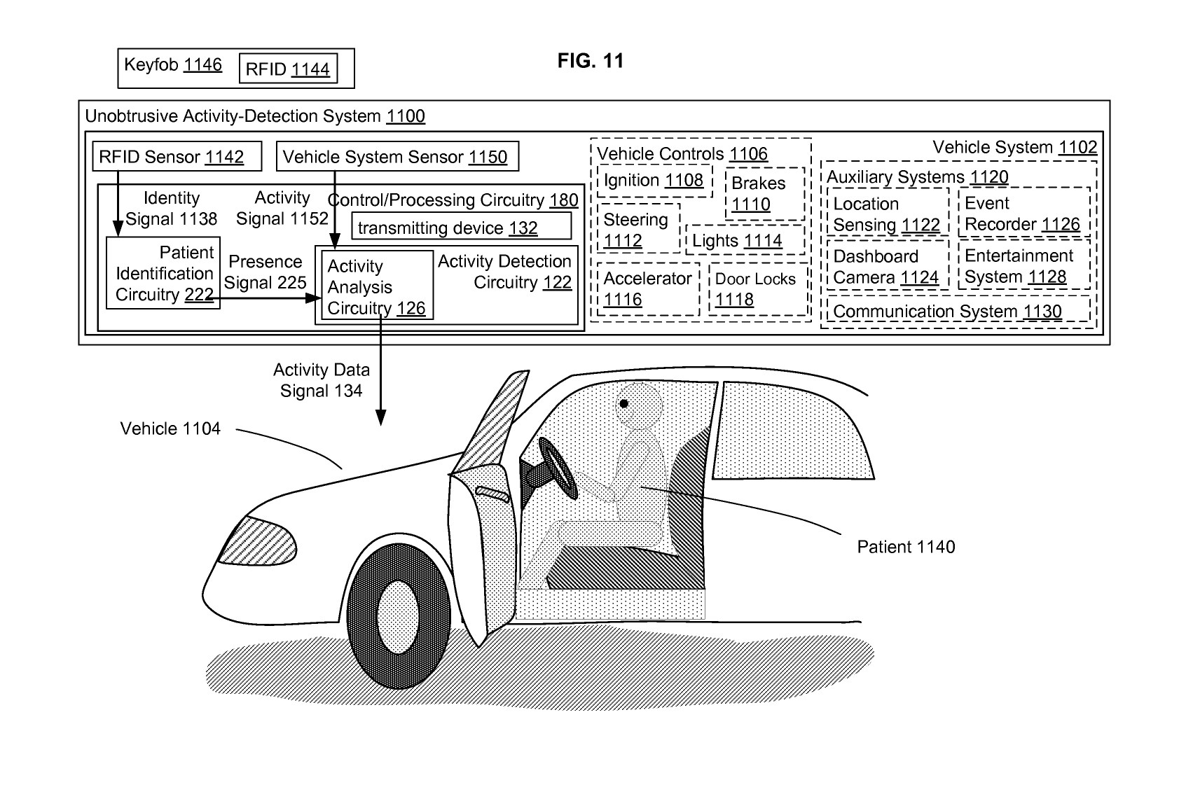

FIG. 11 depicts an example of an unobtrusive activity-detection system 1100 that is incorporated into a vehicle system 1102. Vehicle system 1102 includes one or more components of vehicle 1104, which are built into vehicle 1104 during manufacture or subsequently installed in vehicle 1104. Vehicle system components include vehicle controls 1106 (including, but not limited to ignition 1108, brakes 1110, steering 1112, lights 1114, accelerator 1116, or door locks 1118) and auxiliary systems 1120 (including, but not limited to, location sensing 1122, dashboard camera 1124, event recorder or “black box” 1126 used for tracking vehicle acceleration, deceleration, etc., entertainment system 1128, or communication system 1130). Communication system 1130 may include, for example, a telephone or radio system. The presence and/or identity of patient 1140 in vehicle 1104 is sensed by RFID sensor 1142, which detects RFID 1144 in key fob 1146 carried by patient 1140. Activity of patient 1140 is sensed by one or more vehicle system sensor 1150, including one or more sensors associated with vehicle controls 1106 or auxiliary systems 1120. A wide variety of types of patient activity can be sensed by vehicle system sensor 1150 to provide information regarding the patient's brain-related function. For example, in various aspects patient activity sensed by vehicle system sensor 1150 includes, but is not limited to acceleration, deceleration or steering of vehicle 1104, choice of music, activation/deactivation of lights or door locks, coordination (determined through analysis of video from dashboard cam), choice of location as assessed by location sensing (e.g., GPS) system, etc. In various aspects, rate, frequency, and consistency of sensor activation provide information regarding the patient's mental state. Activity signal 1152 from vehicle system sensor 1150 is provided to activity detection circuitry 122 and activity analysis circuitry 126, which are components of vehicle system 1102, and activity data signal 134 from activity detection circuitry 122 is transmitted by transmitting device 132, which is also a component of a vehicle system 1102.

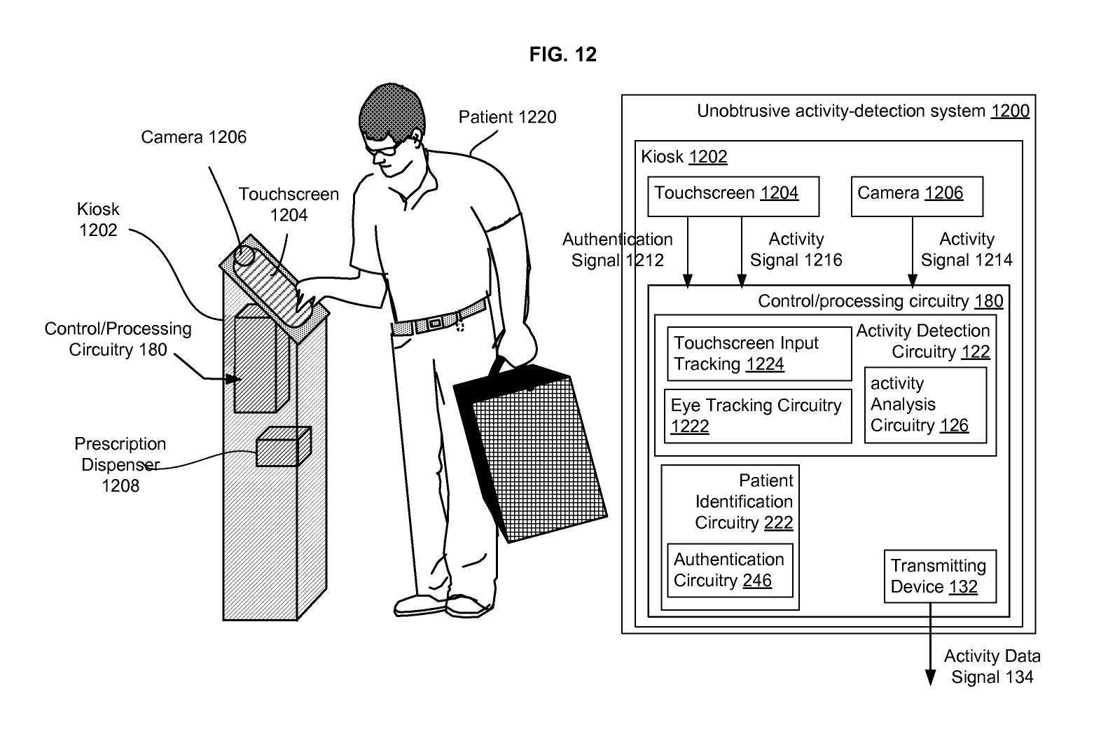

FIG. 12 depicts an example of an unobtrusive activity-detection system 1200 in which activity detection circuitry 122, activity analysis circuitry 126, and transmitting device 132 for transmitting activity data signal 134 to a monitoring location are components of a kiosk 1202 (e.g., as described generally in U.S. Pat. No. 9,135,403 to Tolmosoff, and U.S. Pat. No. 8,996,392 to Cashman et al., both of which are incorporated herein by reference). Kiosk 1202 is a medical kiosk used to provide health-related information, perform medical monitoring (e.g., take a blood pressure reading), dispense medication, and the like. Kiosk 1202 includes a touchscreen 1204, camera 1206, and prescription dispenser 1208. Operation of kiosk 1202 is controlled by control/processing circuitry 180. Patient 1220 signs in to a personal healthcare account via kiosk 1202 by entering a login name and password via touchscreen 1204, by scanning an identification card, or by some other authentication method. Inputs from touchscreen 1204 are processed by touchscreen input tracking 1224. Authentication signal 1212 from touchscreen 1204 (or alternatively, from a card scanner) is provided to authentication circuitry 246 in patient identification circuitry 222. After signing into a personal healthcare account via kiosk 1202, patient 1220 is able to pick up a prescription via prescription dispenser 1208, or perform other healthcare-related activities. While patient 1220 interacts with kiosk 1202 via touchscreen 1204, camera 1206 captures an image of the patient's face, which is provided to control/processing circuitry 180 as a first activity signal 1214. Eye movement has been shown to be indicative of brain-related state, and eye tracking circuitry 1222 is used to track the patient's eye position/direction of gaze and determine the patient's eye movement pattern to assess brain-related state, for example using an approach as described in U.S. Pat. No. 8,808,195 to Tseng et al., which is incorporated herein by reference.

In an aspect, camera 1206 is a smart camera which captures images of the eyes of patient 1202. Image data may include results of visual spectrum imaging, infrared imaging, ultrasound imaging. Smart cameras are commercially available (e.g., Hamamatsu's Intelligent Vision System; http://jp.hamamatsu.com/en/product_info/index.html). Such image capture systems may include dedicated processing elements for each pixel image sensor. Other possible camera systems may include, for example, a pair of infrared charge coupled device cameras to continuously monitor pupil diameter and position. This can be done as the eye follows a moving visual target, and can provide real-time data relating to pupil accommodation relative to objects on a display (e.g., http://jp.hamamatsu.com/en/rd/publication/scientific_american/common/pdf/scientific_06 08.pdf).

Eye movement and/or pupil movement may also be measured by video-based eye tracking circuitry. In these systems, a camera 1206 built into kiosk 1202 focuses on one or both eyes and records eye movement as the viewer looks at a stimulus. Contrast may be used to locate the center of the pupil, and infrared and near-infrared non-collimated light may be used to create a corneal reflection. The vector between these two features can be used to compute gaze intersection with a surface after a calibration for a subject.

Two types of eye tracking techniques include bright pupil eye tracking and dark pupil eye tracking Their difference is based on the location of the illumination source with respect to the optical system. If the illumination is coaxial with the optical path, then the eye acts as a retroreflector as the light reflects off the retina, creating a bright pupil effect similar to red eye. If the illumination source is offset from the optical path, then the pupil appears dark. Thus, in some embodiments, the gaze tracking stimulus source and the gaze response signal sensor are co-aligned. Alternatively, the gaze tracking stimulus source and the gaze response signal sensor may be separately aligned and located.

Bright Pupil tracking creates greater iris/pupil contrast allowing for more robust eye tracking that is less dependent upon iris pigmentation and greatly reduces interference caused by eyelashes and other obscuring features. It also allows for tracking in lighting conditions ranging from total darkness to very bright light. However, bright pupil techniques are not recommended for tracking outdoors as extraneous infrared (IR) sources may interfere with monitoring.

Most eye tracking systems use a sampling rate of at least 30 Hz. Although 50/60 Hz is most common, many video-based eye tracking systems run at 240, 350 or even 1000/1250 Hz, which is recommended in order to capture the detail of the very rapid eye movements during reading, for example.

Eye movements are typically divided into fixations, when the eye gaze pauses in a certain position, and saccades, when the eye gaze moves to another position. A series of fixations and saccades is called a scanpath. Most information from the eye is made available during a fixation, not during a saccade. The central one or two degrees of the visual angle (the fovea) provide the bulk of visual information; input from larger eccentricities (the periphery) generally is less informative. Therefore the locations of fixations along a scanpath indicate what information loci on the stimulus were processed during an eye tracking session. On average, fixations last for around 200 milliseconds during the reading of linguistic text, and 350 milliseconds during the viewing of a scene. Preparing a saccade towards a new goal takes around 200 milliseconds. Scanpaths are useful for analyzing cognitive intent, interest, and salience. Other biological factors (some as simple as gender) may affect the scanpath as well. Eye tracking in human-computer interaction typically investigates the scanpath for usability purposes, or as a method of input in gaze-contingent displays, also known as gaze-based interfaces.

Commercial eye tracking software packages can analyze eye tracking and show the relative probability of eye fixation at particular locations. This allows for a broad analysis of which locations received attention and which ones were ignored. Other behaviors such as blinks, saccades, and cognitive engagement can be reported by commercial software packages. A gaze tracking system for monitoring eye position is available from Seeing Machines Inc., Tucson, Ariz. (see e.g., the Specification Sheet: “faceLAB™ 5 Specifications” which is incorporated herein by reference). Eye position, eye rotation, eye gaze position against screen, pupil diameter and eye vergence distance may be monitored. Eye rotation measurements of up to +/−45 degrees around the y-axis and +/−22 degrees around the x-axis are possible. Typical static accuracy of gaze direction measurement is 0.5-1 degree rotational error.

In addition, in some aspects an image obtained with camera 1206 can be used to determine movement or coordination of the patient. In an aspect, control/processing circuitry 180 includes image processing hardware and/or software used to determine an activity or posture of the subject from an image obtained from camera 1206. Such image processing hardware and/or software may, for example, include or generate a model of the background of the image, segment the image, identify the subject in the image, and analyze the image to determine activity or posture of the subject, e.g., based on parameters such as the angle of the torso relative to the hips, or angle of the shoulders relative to the hips. Processing of an image to determine position or posture-related information may be, for example, as described in U.S. Pat. No. 7,616,779 issued Nov. 10, 2009 to Liau et al., U.S. Pat. No. 8,396,283, issued Mar. 12, 2013 to Iihoshi et al., U.S. Pat. No. 7,330,566, issued Feb. 12, 2008 to Cutler, or U.S. Pat. No. 7,728,839 issued Jun. 1, 2010 to Yang et al., each of which is incorporated herein by reference. In addition, the signal from touchscreen 1204, representing entry of data and instructions via touchscreen 1204 by patient 1220 is used as a second activity signal 1216. Rate, timing, type, and consistency of data entry as assessed through analysis of second activity signal 1216 also provide information regarding the patient's brain-related state. Activity Analysis circuitry 126 combines information from activity signal 1214 and activity signal 1216 to determine compliance of patient 1220 with a prescribed treatment regimen.

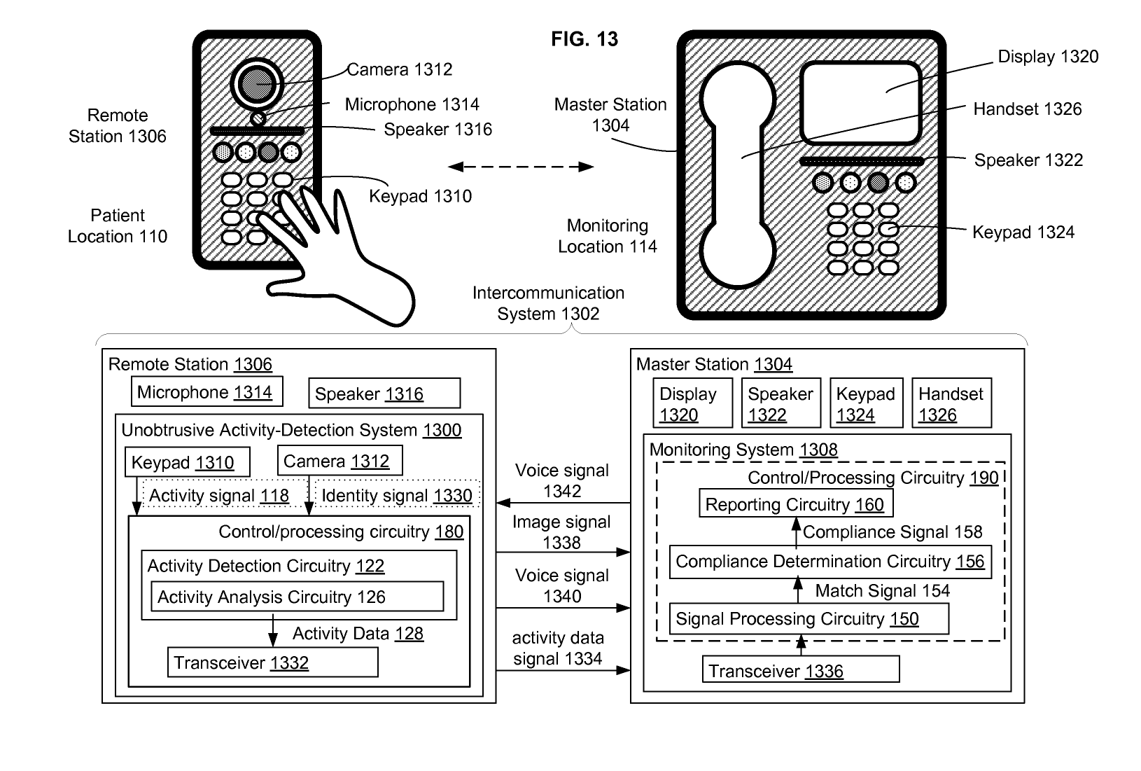

FIG. 13 depicts an example of an unobtrusive activity-detection system 1300 that is incorporated into an intercommunication (“intercom”) system 1302, for example, of the type used with an access control system to control entry of individuals to an apartment building or office building. In an aspect, intercommunication system 1302 includes master station 1304 and at least one remote station 1306. In an aspect, remote station 1306 is an example of a system 108 depicted in FIG. 2, and master station 1304 is an example of a system 112, as depicted in FIG. 4. Master station 1304 is used, for example, at a monitoring location 114 such as the reception desk of the building, where it is monitored by a member of the building staff, for example. Remote station 1306 is used at an entrance to a building to grant access to regular occupants or visitors to the building. This location is considered to be patient location 110 in the situation that remote station 1306 is used to control access of the patient to the building. Remote station 1306 includes keypad 1310, camera 1312, microphone 1314, and speaker 1316. In order to request access to the building, the patient typically presses one or more buttons on keypad 1310. An image of the patient is detected with camera 1312; the patient's voice is sensed with microphone 1314 and speaker 1316 provides for delivery of recorded messages, other notification sounds, or verbal instructions from a building staff person at master station 1304. Master station 1304 includes display 1320 for displaying an image of the patient, speaker 1322 for presenting a voice signal detected with microphone 1314, keypad 1324, and handset 1326 which includes a microphone for sensing a voice signal from the building staff person at master station 1304 to deliver to the patient via speaker 1316. The pattern of entry of an access code, detected via keypad 1310, serves as activity signal 118. Camera 1312 detects an image of the iris of the patient, which serves as identity signal 1330 (i.e., camera 1312 serves as a biometric sensor). Detection of patient presence/identity through biometric analysis can be performed by any of the various approaches described herein above. Activity signal 118 and identity signal 1330 are processed by control/processing circuitry 180, activity detection circuitry 122, activity analysis circuitry 126 to generate activity data 128. Transceiver 1332 transmits activity data signal 1334 to transceiver 1336 in monitoring system 1308. In addition, transceiver 1332 transmits image signal 1338 from camera 1312 and voice signal 1340 from microphone 1314, and receives voice signal 1342, sensed via handset 1326, from master station 1304. Activity data signal 1334 is processed by control/processing circuitry 190, and signal processing circuitry 150, compliance determination circuitry 156 and reporting circuitry 160 as described in connection with FIGS. 1 and 4. Additional data signals and instructions relating to operation of intercommunication system 1302 are sent between remote station 1306 and master station 1304 via transceivers 1332 and 1336, respectively, but are not depicted in FIG. 13.

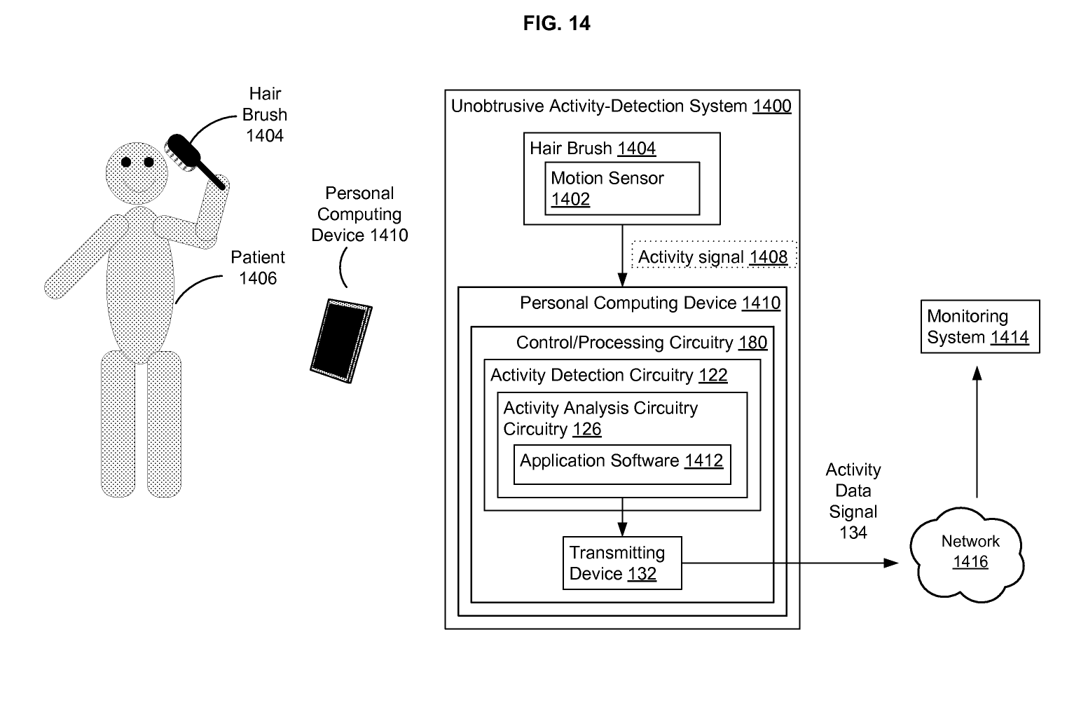

FIG. 14 depicts an example of an unobtrusive activity-detection system 1400 that includes a motion sensor 1402 built into (or, alternatively, attached to) a hair brush 1404 used by patient 1406. In an aspect, motion sensor 1402 is a tri-axial accelerometer. Motion associated with the use of hair brush 1404 is sensed with motion sensor 1402, and an activity signal 1408 is transmitted to personal computing device 1410. (Here, personal computing device 1410 is a tablet computer, but it could alternatively be a cell phone, laptop computer, desktop computer, for example.) Personal computing device 1410 includes control/processing circuitry 180, including activity detection circuitry 122, activity analysis circuitry 126, and transmitting device 132. Application software 1412 configures hardware of personal computing device 1410 to perform functions of activity detection circuitry 122 and activity analysis circuitry 126. Transmitting device 132 transmits activity data signal 134 to monitoring system 1414 via network 1416. In an aspect, activity data signal 134 includes information regarding the time of day at which hair brush 1404 was used and how long it was used for. In many cases, this will provide sufficient information regarding use of hair brush 1404 by patient 1406. However, information relating to the nature of movement sensed—e.g., was the movement weak or vigorous, erratic or regular, was any tremor detected, etc. may also be sensed and may provide additional information regarding the brain-related functioning of patient 1406. In another aspect, motion sensor 1402 or other activity sensor, activity detection circuitry 122, activity analysis circuitry 126, and transmitting device 132 are all components of a personal item such as hair brush 1404.

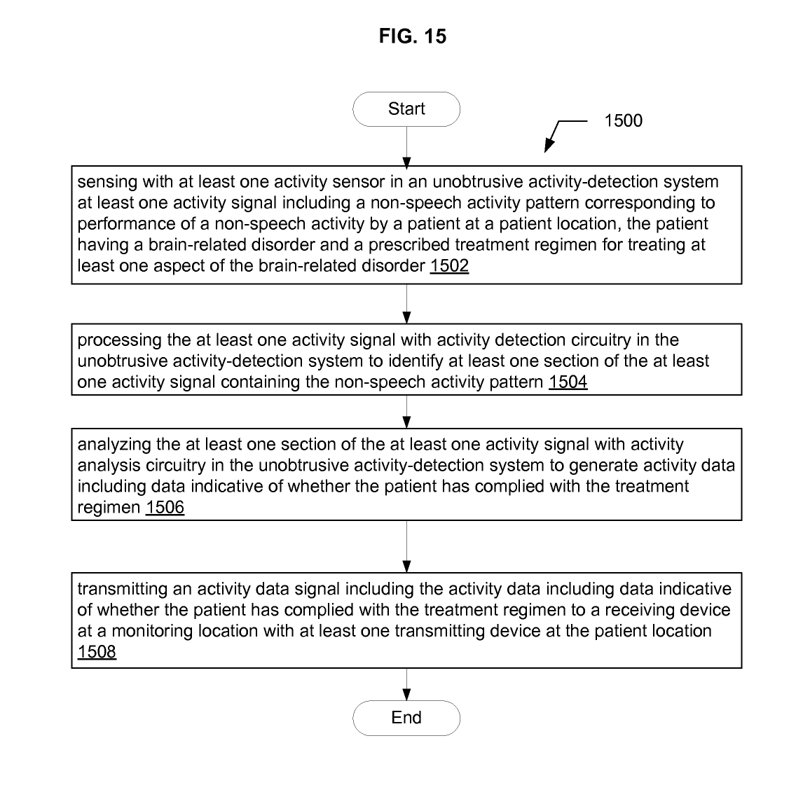

FIG. 15 is a flow diagram of a method 1500 relating to monitoring compliance of a patient with a prescribed treatment regimen. Method 1500 includes sensing with at least one activity sensor in an unobtrusive activity-detection system at least one activity signal including a non-speech activity pattern corresponding to performance of a non-speech activity by a patient at a patient location, the patient having a brain-related disorder and a prescribed treatment regimen for treating at least one aspect of the brain-related disorder, as indicated at 1502; processing the at least one activity signal with activity detection circuitry in the unobtrusive activity-detection system to identify at least one section of the at least one activity signal containing the non-speech activity pattern, as indicated at 1504; analyzing the at least one section of the at least one activity signal with activity analysis circuitry in the unobtrusive activity-detection system to generate activity data including data indicative of whether the patient has complied with the treatment regimen, as indicated at 1506; and transmitting an activity data signal including the activity data including data indicative of whether the patient has complied with the treatment regimen to a receiving device at a monitoring location with at least one transmitting device at the patient location, as indicated at 1508. In various aspects, method 1500 is carried out with unobtrusive activity detection system 108 as depicted in FIGS. 1, 2 and 3, for example.

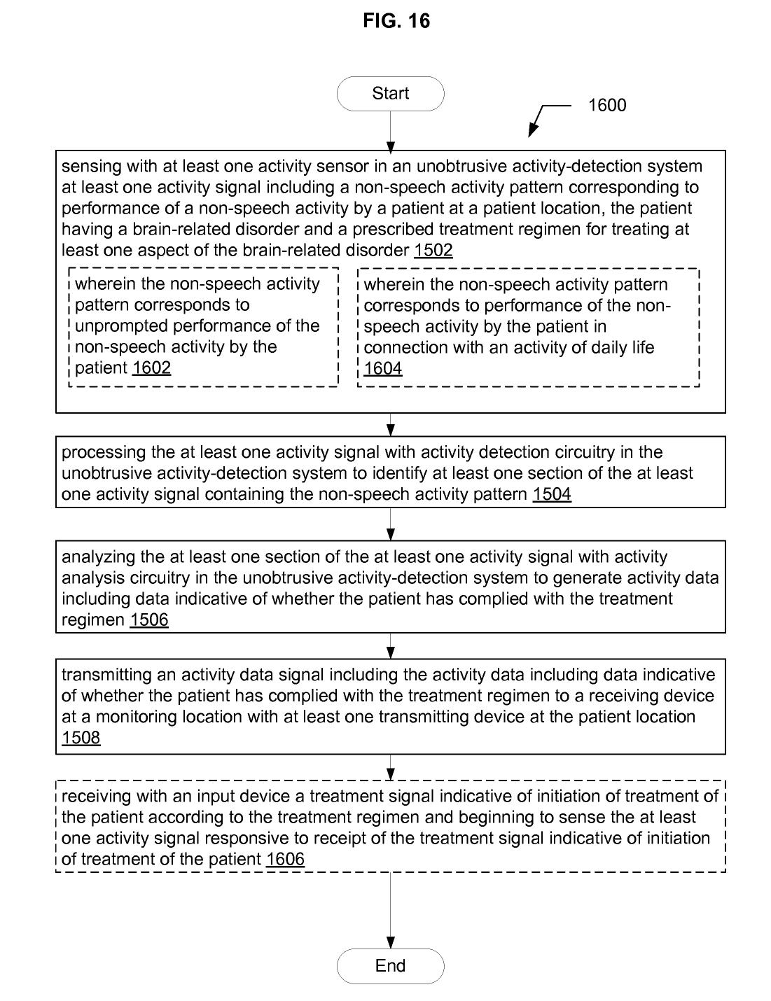

FIGS. 16-28 depict variations and expansions of method 1500 as shown in FIG. 15. In the methods depicted in FIGS. 16-28, steps 1502-1508 are as described generally in connection with FIG. 15. Here and elsewhere, method steps outlined with dashed lines represent steps that are included in some, but not all method aspects, and combinations of steps other than those specifically depicted in the figures are possible as would be known by those having ordinary skill in the relevant art.

FIG. 16 depicts method 1600, which includes steps 1502-1508 as described above. As indicated at 1602, in an aspect the non-speech activity pattern corresponds to unprompted performance of the non-speech activity by the patient. As indicated at 1604, in another aspect, the non-speech activity pattern corresponds to performance of the non-speech activity by the patient in connection with an activity of daily life. Examples of “activities of daily life” are listed herein above.

In an aspect, method 1600 includes receiving with an input device a treatment signal indicative of initiation of treatment of the patient according to the treatment regimen and beginning to sense the at least one activity signal responsive to receipt of the treatment signal indicative of initiation of treatment of the patient, as indicated at 1606. See, e.g., treatment signal 220 in FIG. 2.

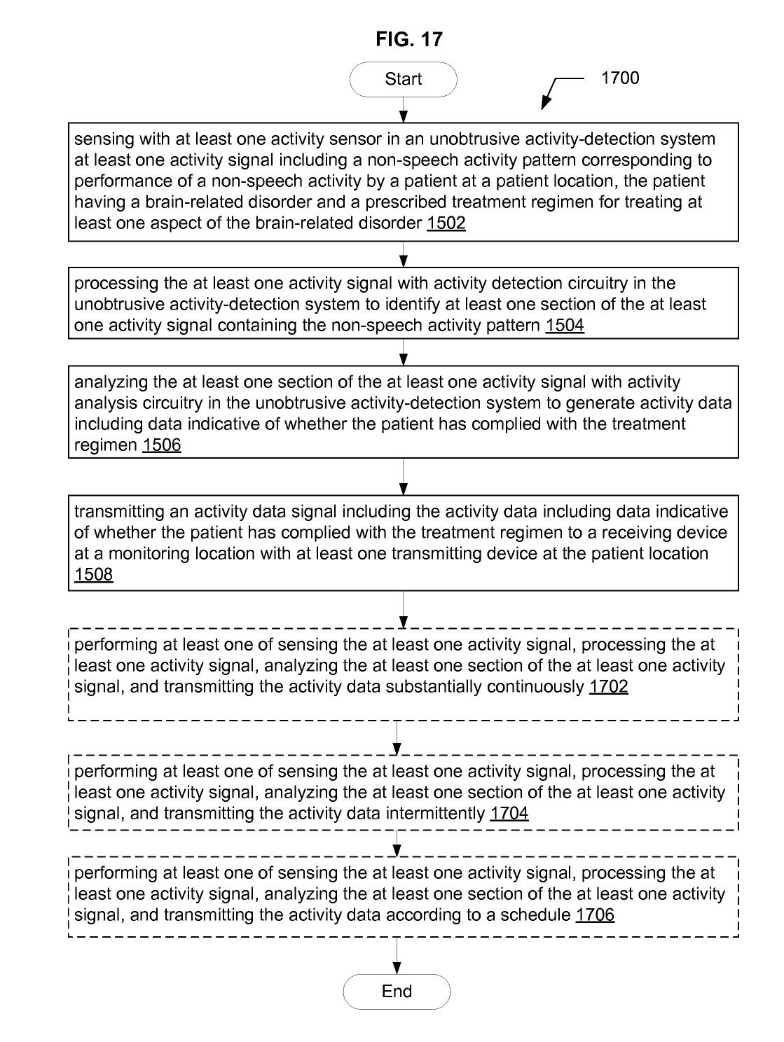

FIG. 17 depicts method 1700, which includes steps 1502-1508 as described in connection with FIG. 15. In an aspect, method 1700 includes performing at least one of sensing the at least one activity signal, processing the at least one activity signal, analyzing the at least one section of the at least one activity signal, and transmitting the activity data substantially continuously, as indicated at 1702. In another aspect, method 1700 includes performing at least one of sensing the at least one activity signal, processing the at least one activity signal, analyzing the at least one section of the at least one activity signal, and transmitting the activity data intermittently, as indicated at 1704. In another aspect, method 1700 includes performing at least one of sensing the at least one activity signal, processing the at least one activity signal, analyzing the at least one section of the at least one activity signal, and transmitting the activity data according to a schedule, as indicated at 1706.

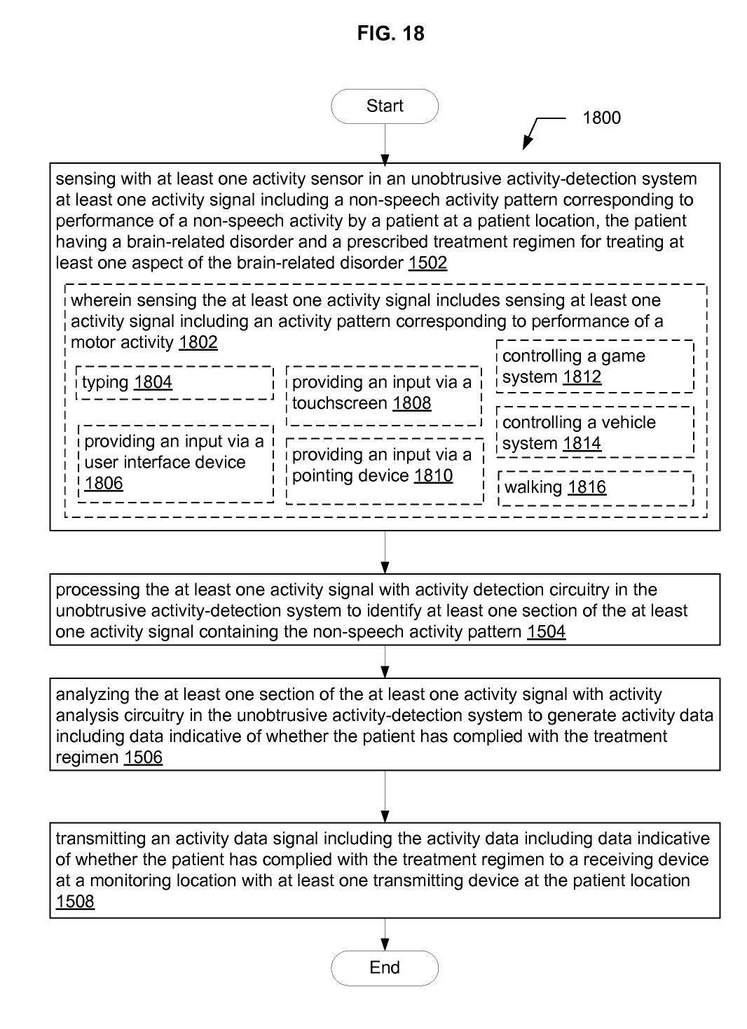

FIG. 18 depicts method 1800, wherein sensing the at least one activity signal includes sensing at least one activity signal including an activity pattern corresponding to performance of a motor activity, as indicated at 1802. In various aspects, the motor activity includes typing, as indicated at 1804; providing an input via a user interface device, as indicated at 1806; providing an input via a touchscreen, as indicated at 1808; providing an input via a pointing device, as indicated at 1810; controlling a game system, as indicated at 1812; controlling a vehicle system, as indicated at 1814; or walking, as indicated at 1816.

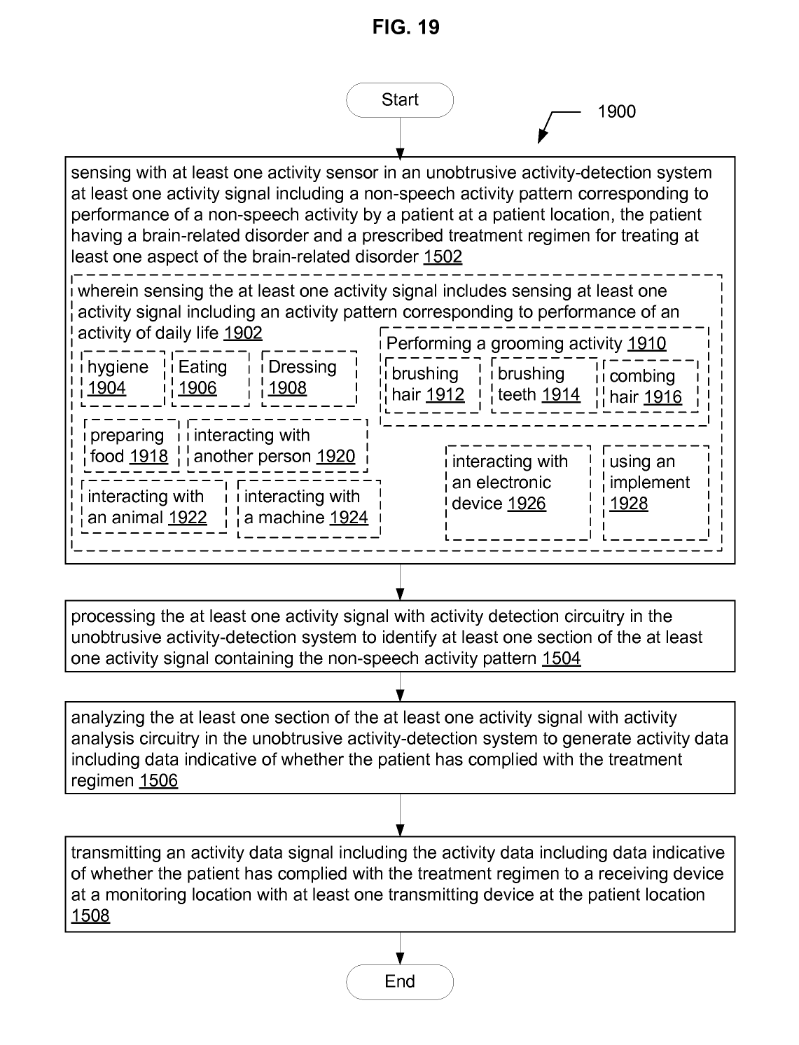

FIG. 19 depicts a method 1900, wherein sensing the at least one activity signal includes sensing at least one activity signal including an activity pattern corresponding to performance of an activity of daily life, as indicated at 1902. In various aspects, the activity of daily life includes at least one of hygiene, as indicated at 1904; eating, as indicated at 1906; dressing, as indicated at 1908; performing a grooming activity, as indicated at 1910 (e.g., brushing hair, as indicated at 1912; brushing teeth, as indicated at 1914; or combing hair, as indicated at 1916); preparing food, as indicated at 1918; interacting with another person, as indicated at 1920; interacting with an animal, as indicated at 1922; interacting with a machine, as indicated at 1924; interacting with an electronic device, as indicated at 1926; or using an implement, as indicated at 1928.

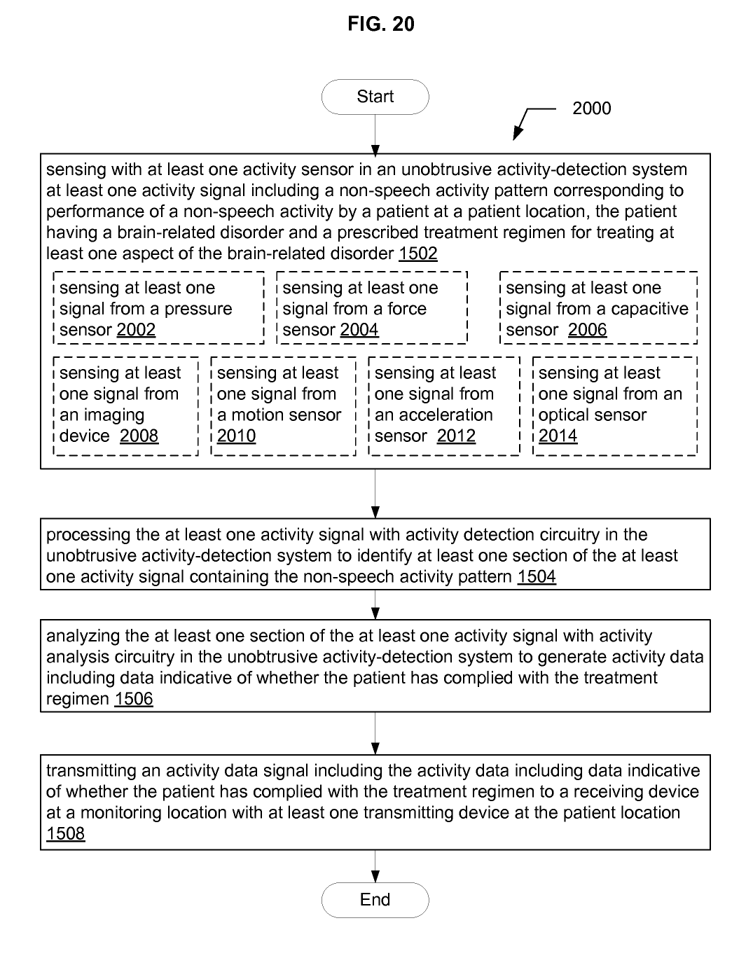

FIG. 20 depicts a method 2000, wherein, in various aspects, sensing the at least one activity signal includes sensing at least one signal from a pressure sensor, as indicated at 2002; a force sensor, as indicated at 2004; a capacitive sensor, as indicated at 2006; an imaging device, as indicated at 2008; a motion sensor, as indicated at 2010; an acceleration sensor, as indicated at 2012; or an optical sensor, as indicated at 2014.

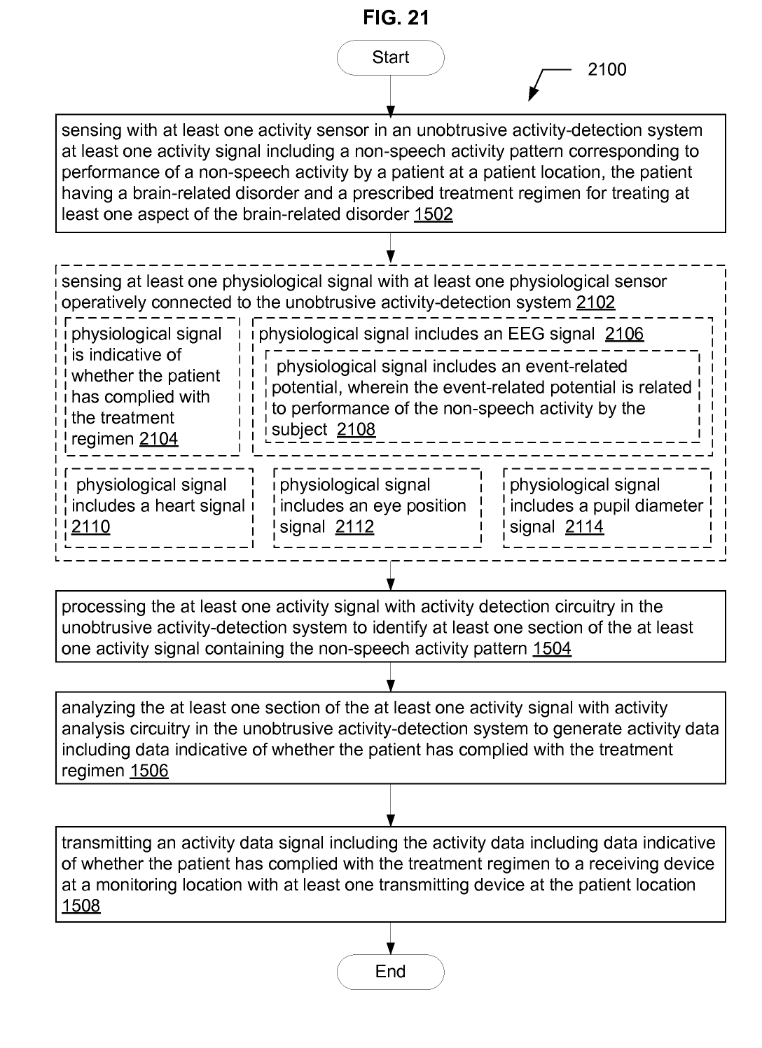

FIG. 21 depicts a method 2100, which includes sensing at least one physiological signal with at least one physiological sensor operatively connected to the unobtrusive activity-detection system, as indicated at 2102. For example, in an aspect the at least one physiological signal is indicative of whether the patient has complied with the treatment regimen, as indicated at 2104. In an aspect, the at least one physiological signal includes an EEG signal, as indicated at 2106. For example, in an aspect the at least one physiological signal includes an event-related potential, wherein the event-related potential is related to performance of the non-speech activity by the subject, as indicated at 2108. In other aspects, the at least one physiological signal includes one or more of a heart signal, as indicated at 1220; an eye position signal, as indicated at 2112; or a pupil diameter signal, as indicated at 2114.

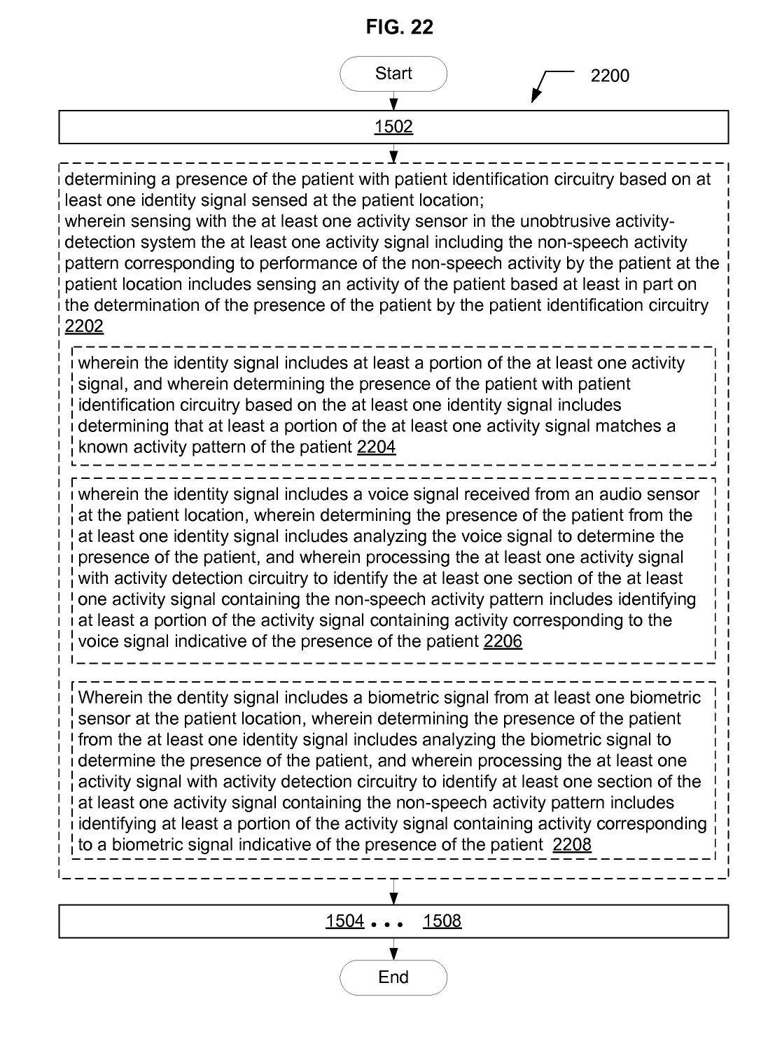

FIG. 22 depicts a method 2200, which includes determining a presence of the patient with patient identification circuitry based on at least one identity signal sensed at the patient location, wherein sensing with the at least one activity sensor in the unobtrusive activity-detection system the at least one activity signal including the non-speech activity pattern corresponding to performance of the non-speech activity by the patient at the patient location includes sensing an activity of the patient based at least in part on the determination of the presence of the patient by the patient identification circuitry, as indicated 2202. In an aspect, the identity signal includes at least a portion of the at least one activity signal, and determining the presence of the patient with patient identification circuitry based on the at least one identity signal includes determining that at least a portion of the at least one activity signal matches a known activity pattern of the patient, as indicated at 2204. In another aspect, the identity signal includes a voice signal received from an audio sensor at the patient location, and determining the presence of the patient from the at least one identity signal includes analyzing the voice signal to determine the presence of the patient, and wherein processing the at least one activity signal with activity detection circuitry to identify the at least one section of the at least one activity signal containing the non-speech activity pattern includes identifying at least a portion of the activity signal containing activity corresponding to the voice signal indicative of the presence of the patient, as indicated at 2206.

In another aspect, as indicated at 2208, the identity signal includes a biometric signal from at least one biometric sensor at the patient location, wherein determining the presence of the patient from the at least one identity signal includes analyzing the biometric signal to determine the presence of the patient, and wherein processing the at least one activity signal with activity detection circuitry to identify at least one section of the at least one activity signal containing the non-speech activity pattern includes identifying at least a portion of the activity signal containing activity corresponding to a biometric signal indicative of the presence of the patient.

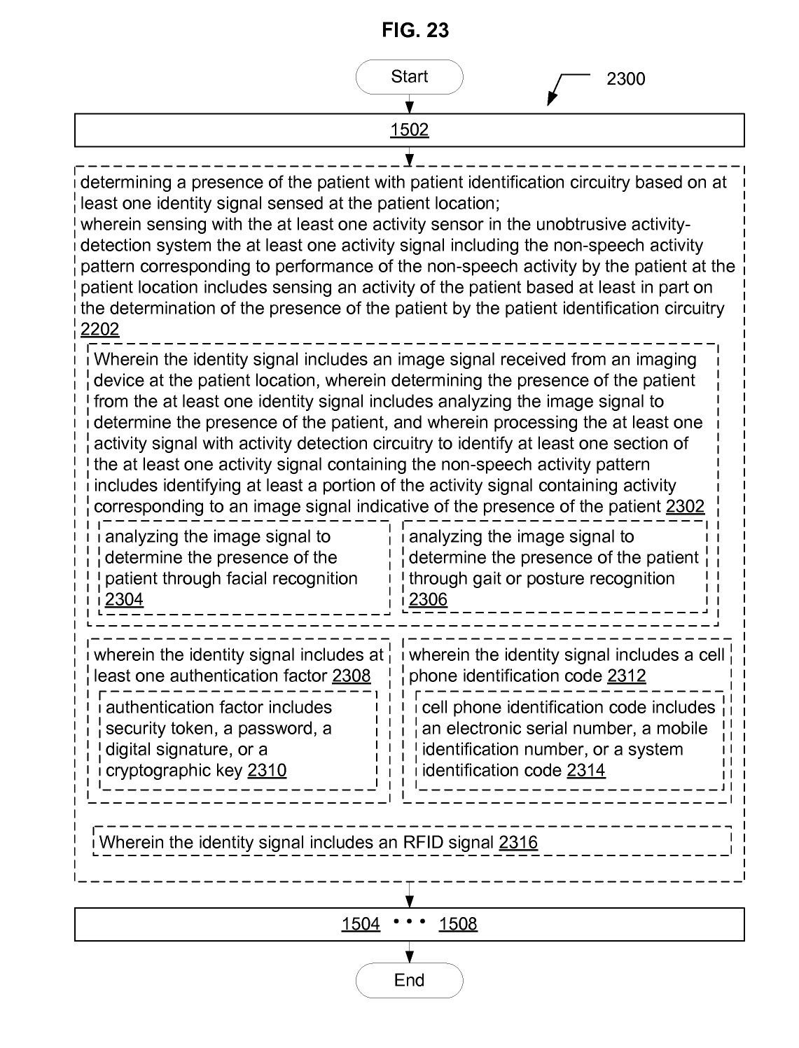

FIG. 23 is a flow diagram showing further aspects of the method shown in FIG. 22. Method 2300, shown in FIG. 23, includes step 1502-1508, as described herein above, as well as step 2202, which is described in connection with FIG. 22. In addition, in method 2300, the identity signal includes an image signal received from an imaging device at the patient location, wherein determining the presence of the patient from the at least one identity signal includes analyzing the image signal to determine the presence of the patient, and wherein processing the at least one activity signal with activity detection circuitry to identify at least one section of the at least one activity signal containing the non-speech activity pattern includes identifying at least a portion of the activity signal containing activity corresponding to an image signal indicative of the presence of the patient, as indicated at 2302. Method 2300 includes analyzing the image signal to determine the presence of the patient through facial recognition, as indicated at 2304, or analyzing the image signal to determine the presence of the patient through gait or posture recognition, as indicated at 2306.

In other aspects, the identity signal includes at least one authentication factor, as indicated at 2308 (for example, a security token, a password, a digital signature, or a cryptographic key, as indicated at 2310), or a cell phone identification code, as indicated at 2312 (for example, an electronic serial number, a mobile identification number, or system identification code, as indicated at 2314). In yet other aspects, the identity signal includes an RFID signal, as indicated at 2316.

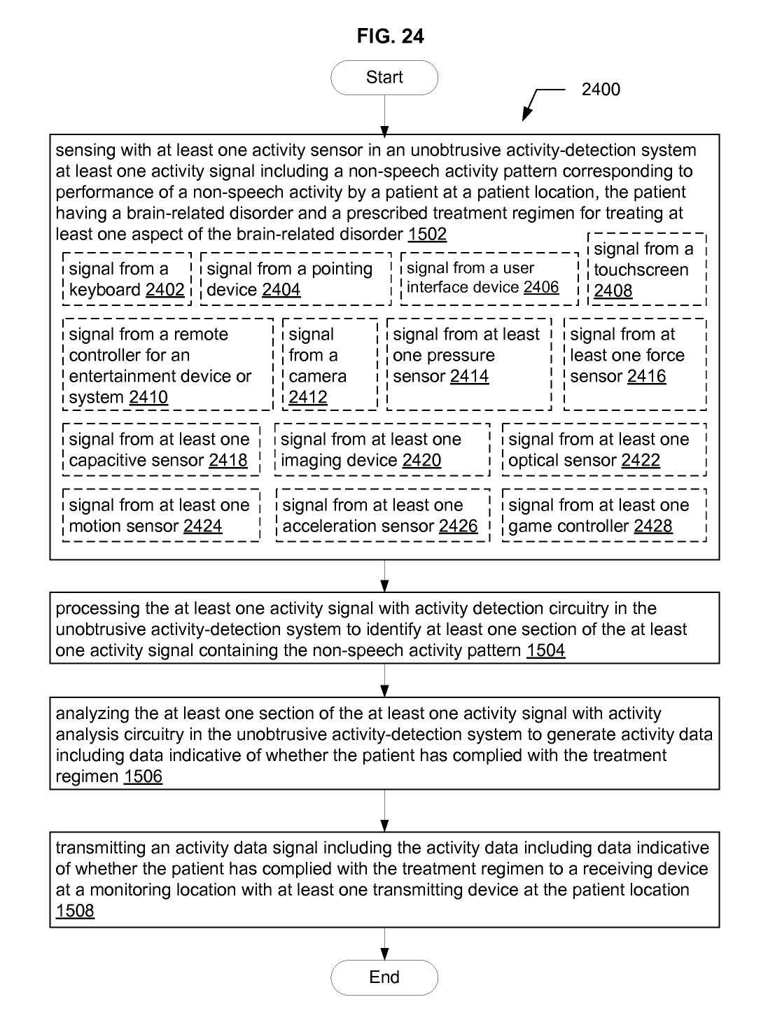

FIG. 24 depicts further aspects of a method 2400 relating to sensing of the activity signal. For example, in various aspects, the at least one activity signal includes a signal from a keyboard, as indicated at 2402; a signal from a pointing device, as indicated at 2404; a signal from a user interface device, as indicated at 2406; a signal from a touchscreen, as indicated at 2408; a signal from a remote controller for an entertainment device or system, as indicated at 2410; a signal from a camera, as indicated at 2412; a signal from at least one pressure sensor, as indicated at 2414; a signal from at least one force sensor, as indicated at 2416; a signal from at least one capacitive sensor, as indicated at 2418; a signal from at least one imaging device, as indicated at 2420; a signal from at least one optical sensor, as indicated at 2422; a signal from at least one motion sensor, as indicated at 2424; a signal from at least one acceleration sensor, as indicated at 2426; or a signal from at least one game controller, as indicated at 2428.

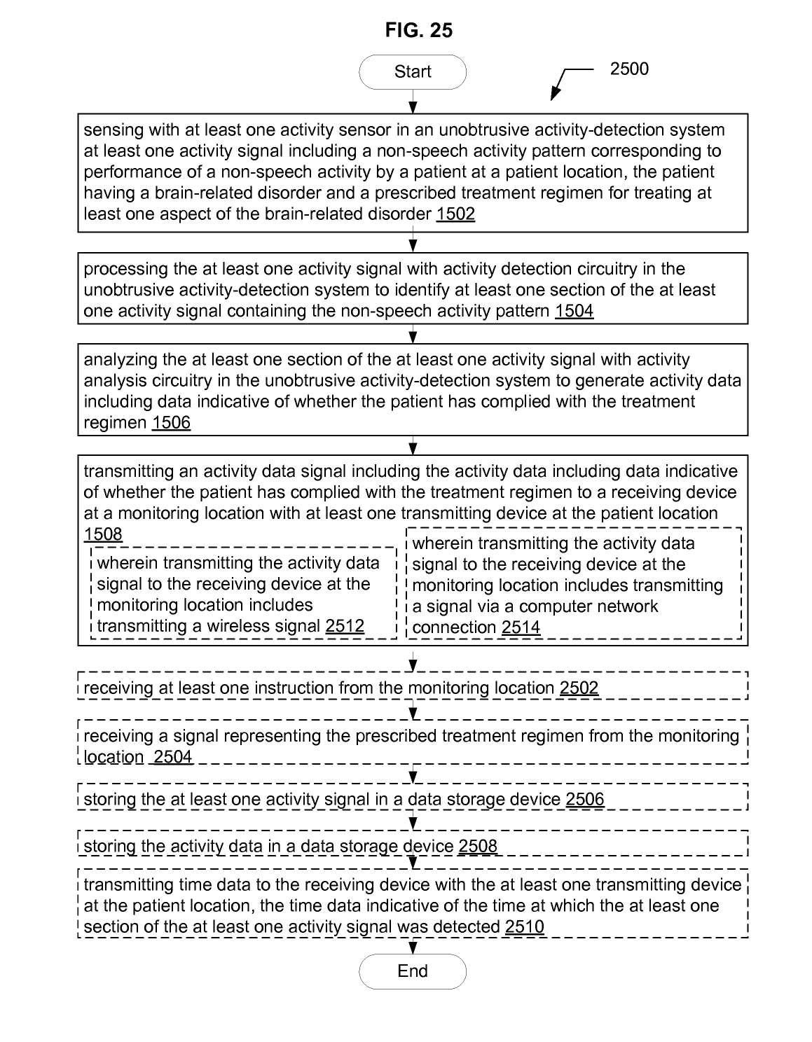

FIG. 25 shows various other method aspects. For example, in an aspect, a method 2500 includes receiving at least one instruction from the monitoring location, as indicated at 2502; receiving a signal representing the prescribed treatment regimen from the monitoring location, as indicated at 2504; storing the at least one activity signal in a data storage device, as indicated at 2506; storing the activity data in a data storage device, as indicated at 2508; or transmitting time data to the receiving device with the at least one transmitting device at the patient location, the time data indicative of the time at which the at least one section of the at least one activity signal was detected, as indicated at 2510. In an aspect, transmitting the activity data signal to the receiving device at the monitoring location includes transmitting a wireless signal, as indicated at 2512. In another aspect, transmitting the activity data signal to the receiving device at the monitoring location includes transmitting a signal via a computer network connection, as indicated at 2514.

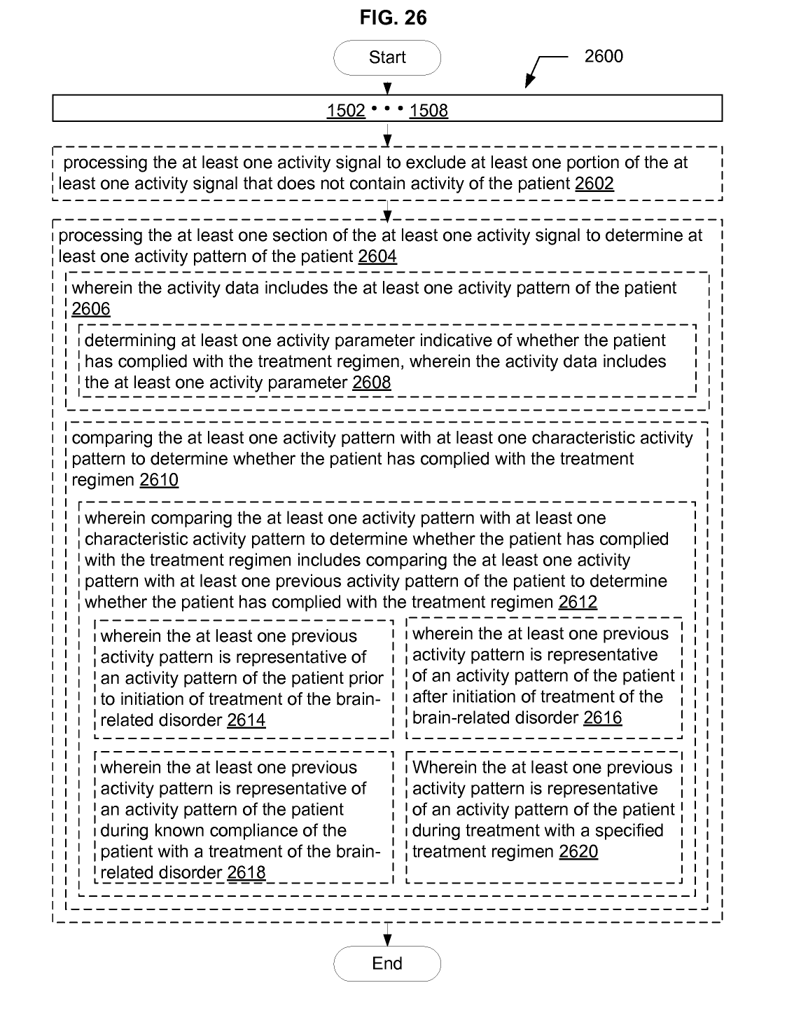

FIG. 26 depicts a method 2600. In an aspect, method 2600 includes processing the at least one activity signal to exclude at least one portion of the at least one activity signal that does not contain activity of the patient, as indicated at 2602. In another aspect, method 2600 includes processing the at least one section of the at least one activity signal to determine at least one activity pattern of the patient, as indicated at 2604. In an aspect, the activity data includes the at least one activity pattern of the patient, as indicated at 2606. For example, in an aspect method 2600 includes determining at least one activity parameter indicative of whether the patient has complied with the treatment regimen, wherein the activity data includes the at least one activity parameter, as indicated at 2608.

In some aspects, method 2600 includes comparing the at least one activity pattern with at least one characteristic activity pattern to determine whether the patient has complied with the treatment regimen, as indicated at 2610. For example, in an aspect comparing the at least one activity pattern with at least one characteristic activity pattern to determine whether the patient has complied with the treatment regimen includes comparing the at least one activity pattern with at least one previous activity pattern of the patient to determine whether the patient has complied with the treatment regimen, as indicated at 2612. For example, in various aspects, the at least one previous activity pattern is representative of an activity pattern of the patient prior to initiation of treatment of the brain-related disorder, as indicated at 2614; an activity pattern of the patient after initiation of treatment of the brain-related disorder, as indicated at 2616; an activity pattern of the patient during known compliance of the patient with a treatment of the brain-related disorder, as indicated at 2618; and an activity pattern of the patient during treatment with a specified treatment regimen, as indicated at 2620.

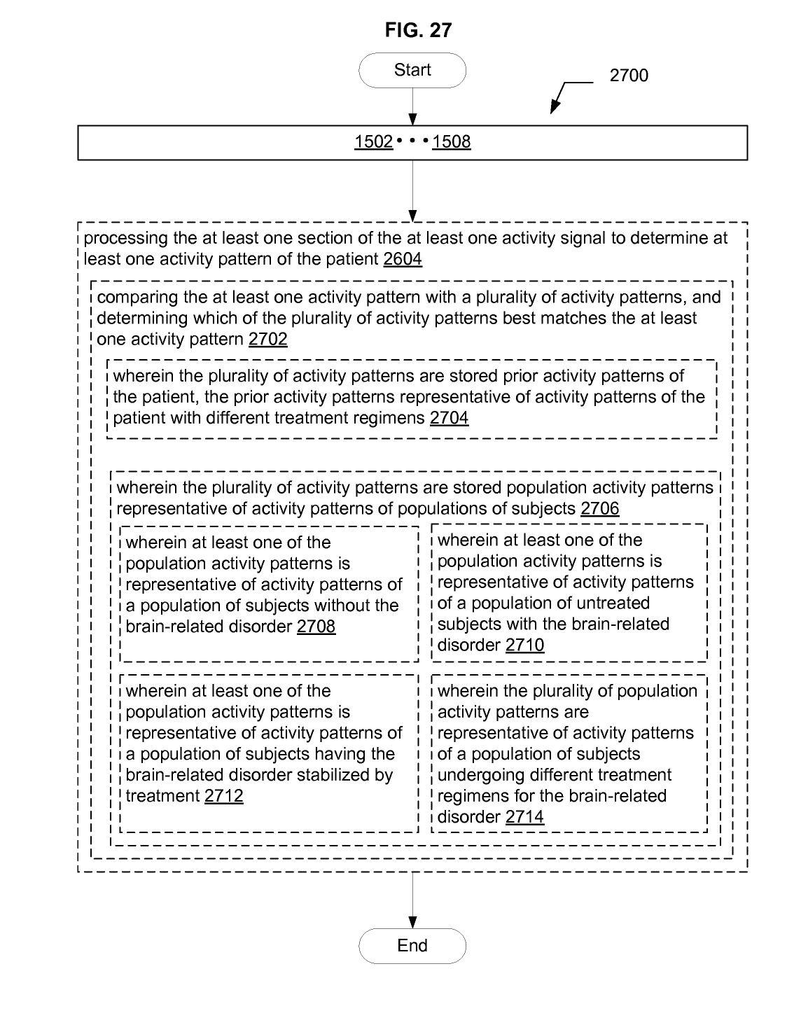

FIG. 27 depicts aspects of a method 2700, showing further aspects of step 2604 as shown in FIG. 26. In an aspect, method 2700 includes comparing the at least one activity pattern with a plurality of activity patterns, and determining which of the plurality of activity patterns best matches the at least one activity pattern, as indicated at 2702. In an aspect, the plurality of activity patterns are stored prior activity patterns of the patient, and the prior activity patterns are representative of activity patterns of the patient with different treatment regimens, as indicated at 2704. In another aspect, the plurality of activity patterns are stored population activity patterns representative of activity patterns of populations of subjects, as indicated at 2706. For example, in various aspects, at least one of the population activity patterns is representative of activity patterns of a population of subjects without the brain-related disorder, as indicated at 2708; activity patterns of a population of untreated subjects with the brain-related disorder, as indicated at 2710; activity patterns of a population of subjects having the brain-related disorder stabilized by treatment, as indicated at 2712; or activity patterns of a population of subjects undergoing different treatment regimens for the brain-related disorder, as indicated at 2714.

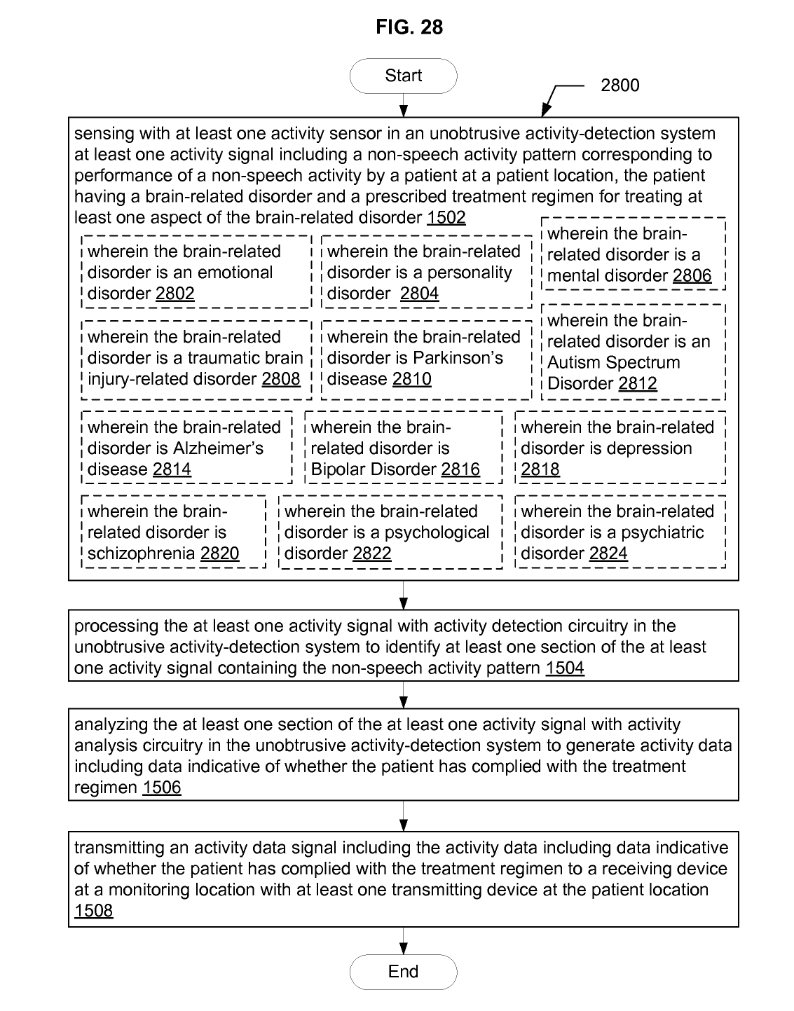

FIG. 28 depicts a method 2800. In various aspects, the brain-related disorder is an emotional disorder, as indicated at 2802; a personality disorder, as indicated at 2804; a mental disorder, as indicated at 2806; a traumatic brain injury-related disorder, as indicated at 2808; Parkinson's disease, as indicated at 2810; an Autism Spectrum Disorder, as indicated at 2812; Alzheimer's disease, as indicated at 2814; Bipolar Disorder, as indicated at 2816; depression, as indicated at 2828; schizophrenia, as indicated at 2820; a psychological disorder, as indicated at 2822; or a psychiatric disorder, as indicated at 2824.

As noted above, in some aspects, a brain-related disorder is a mental disorder, psychological disorder, or psychiatric disorder. A mental disorder, psychological disorder, or psychiatric disorder can include, for example, a psychological pathology, psychopathology, psychosocial pathology, social pathology, or psychobiology disorder. A mental disorder, psychological disorder, or psychiatric disorder can be any disorder categorized in any Diagnostic and Statistical Manual (DSM) or International Statistical Classification of Diseases (ICD) Classification of Mental and Behavioural Disorders text, and may be, for example and without limitation, a neurodevelopmental disorder (e.g., autism spectrum disorder or attention-deficit/hyperactivity disorder), a psychotic disorder (e.g., schizophrenia), a mood disorder, a bipolar disorder, a depressive disorder, an anxiety disorder, an obsessive-compulsive disorder, a trauma- or stressor-related disorder, a dissociative disorder, a somatic symptom disorder, an eating disorder, an impulse-control disorder, a substance-related or addictive disorder, a personality disorder (e.g., narcissistic personality disorder or antisocial personality disorder), a neurocognitive disorder, a major or mild neurocognitive disorder (e.g., one due to Alzheimer's disease, traumatic brain injury, HIV infection, prion disease, Parkinson's disease, Huntington's disease, or substance/medication). A mental disorder, psychological disorder, or psychiatric disorder can be any disorder described by the NIH National Institute of Mental Health (NIMH) Research Domain Criteria Project and may include a biological disorder involving brain circuits that implicate specific domains of cognition, emotion, or behavior. In an aspect, a brain-related disorder includes a serious mental illness or serious emotional disturbance.

In various aspects, a brain-related disorder includes a serious mental illness or serious emotional disturbance, a mental disorder, psychological disorder, or psychiatric disorder.

In an aspect, a brain disorder is a traumatic disorder, such as a traumatic brain injury. Traumatic brain injury-induced disorders may present with dysfunction in cognition, communication, behavior, depression, anxiety, personality changes, aggression, acting out, or social inappropriateness. See, e.g., Jeffrey Nicholl and W. Curt LaFrance, Jr., “Neuropsychiatric Sequelae of Traumatic Brain Injury,” Semin Neurol. 2009, 29(3):247-255.

In an aspect, a brain-related disorder is a lesion-related disorder. A brain lesion can include, for example and without limitation, a tumor, an aneurysm, ischemic damage (e.g., from stroke), an abscess, a malformation, inflammation, or any damage due to trauma, disease, or infection. An example of a lesion-related disorder is a disorder associated with a right-hemisphere lesion.

In an aspect, a brain disorder is a neurological disorder. A neurological disorder may be, for example and without limitation, Alzheimer's disease, a brain tumor, a developmental disorder, epilepsy, a neurogenetic disorder, Parkinson's disease, Huntington's disease, a neurodegenerative disorder, stroke, traumatic brain injury or a neurological consequence of AIDS. Neurological disorders are described on the website of the National Institutes of Health (NIH) National Institute of Neurological Disorders and Stroke (NINDS).

In various embodiments, methods as described herein may be performed according to instructions implementable in hardware, software, and/or firmware. Such instructions may be stored in non-transitory machine-readable data storage media, for example. Those having skill in the art will recognize that the state of the art has progressed to the point where there is little distinction left between hardware, software, and/or firmware implementations of aspects of systems; the use of hardware, software, and/or firmware is generally (but not always, in that in certain contexts the choice between hardware and software can become significant) a design choice representing cost vs. efficiency tradeoffs. Those having skill in the art will appreciate that there are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; alternatively, if flexibility is paramount, the implementer may opt for a mainly software implementation; or, yet again alternatively, the implementer may opt for some combination of hardware, software, and/or firmware in one or more machines, compositions of matter, and articles of manufacture. Hence, there are several possible vehicles by which the processes and/or devices and/or other technologies described herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary. Those skilled in the art will recognize that optical aspects of implementations will typically employ optically oriented hardware, software, and or firmware.

In some implementations described herein, logic and similar implementations may include software or other control structures. Electrical circuitry, for example, may have one or more paths of electrical current constructed and arranged to implement various functions as described herein. In some implementations, one or more media may be configured to bear a device-detectable implementation when such media hold or transmit device detectable instructions operable to perform as described herein. In some variants, for example, implementations may include an update or modification of existing software or firmware, or of gate arrays or programmable hardware, such as by performing a reception of or a transmission of one or more instructions in relation to one or more operations described herein. Alternatively or additionally, in some variants, an implementation may include special-purpose hardware, software, firmware components, and/or general-purpose components executing or otherwise invoking special-purpose components.

Implementations may include executing a special-purpose instruction sequence or invoking circuitry for enabling, triggering, coordinating, requesting, or otherwise causing one or more occurrences of virtually any functional operations described herein. In some variants, operational or other logical descriptions herein may be expressed as source code and compiled or otherwise invoked as an executable instruction sequence. In some contexts, for example, implementations may be provided, in whole or in part, by source code, such as C++, or other code sequences. In other implementations, source or other code implementation, using commercially available and/or techniques in the art, may be compiled/implemented/translated/converted into a high-level descriptor language (e.g., initially implementing described technologies in C or C++ programming language and thereafter converting the programming language implementation into a logic-synthesizable language implementation, a hardware description language implementation, a hardware design simulation implementation, and/or other such similar mode(s) of expression). For example, some or all of a logical expression (e.g., computer programming language implementation) may be manifested as a Verilog-type hardware description (e.g., via Hardware Description Language (HDL) and/or Very High Speed Integrated Circuit Hardware Descriptor Language (VHDL)) or other circuitry model which may then be used to create a physical implementation having hardware (e.g., an Application Specific Integrated Circuit). Those skilled in the art will recognize how to obtain, configure, and optimize suitable transmission or computational elements, material supplies, actuators, or other structures in light of these teachings.

This detailed description sets forth various embodiments of devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In an embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one having skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to non-transitory machine-readable data storage media such as a recordable type medium such as a floppy disk, a hard disk drive, a Compact Disc (CD), a Digital Video Disk (DVD), a digital tape, a computer memory, etc. A signal bearing medium may also include transmission type medium such as a digital and/or an analog communication medium (e.g., a fiber optic cable, a waveguide, a wired communications link, a wireless communication link (e.g., transmitter, receiver, transmission logic, reception logic, etc.) and so forth).

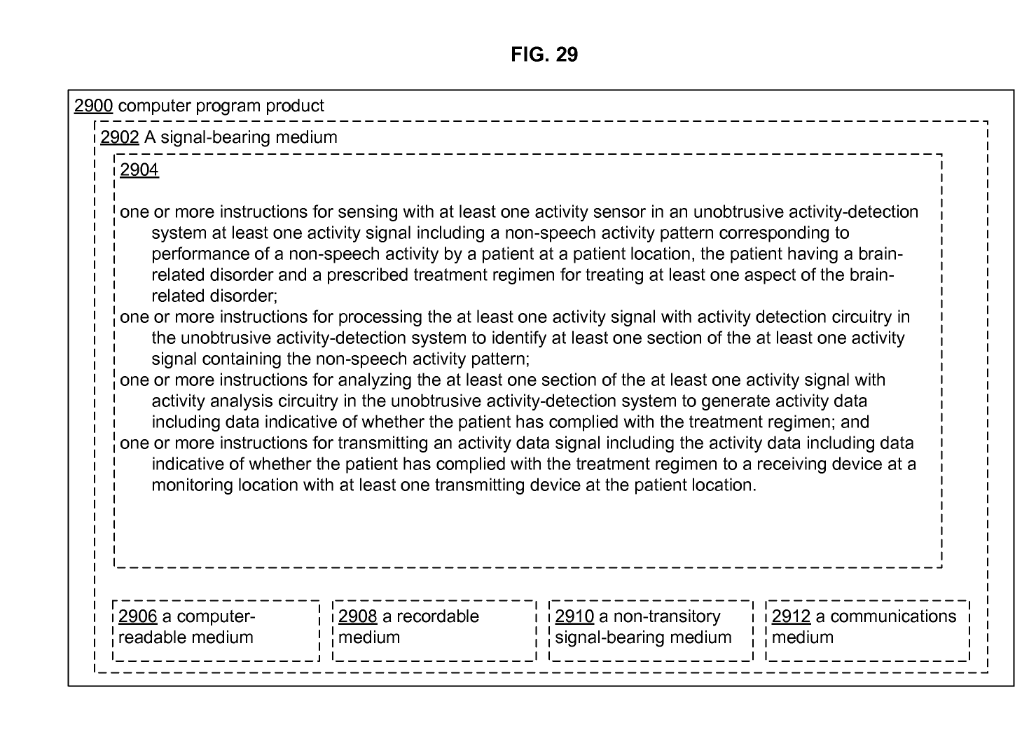

FIG. 29 is a block diagram of a computer program product 2900 for implementing a method as described in connection with FIG. 15. Computer program product 2900 includes a signal-bearing medium 2902 bearing one or more instructions for sensing with at least one activity sensor in an unobtrusive activity-detection system at least one activity signal including a non-speech activity pattern corresponding to performance of a non-speech activity by a patient at a patient location, the patient having a brain-related disorder and a prescribed treatment regimen for treating at least one aspect of the brain-related disorder; one or more instructions for processing the at least one activity signal with activity detection circuitry in the unobtrusive activity-detection system to identify at least one section of the at least one activity signal containing the non-speech activity pattern; one or more instructions for analyzing the at least one section of the at least one activity signal with activity analysis circuitry in the unobtrusive activity-detection system to generate activity data including data indicative of whether the patient has complied with the treatment regimen; and one or more instructions for transmitting an activity data signal including the activity data including data indicative of whether the patient has complied with the treatment regimen to a receiving device at a monitoring location with at least one transmitting device at the patient location, as indicated at 2904. Signal-bearing medium 2902 may be, for example, a computer-readable medium 2906, a recordable medium 2908, a non-transitory signal-bearing medium 2910, or a communications medium 2912, examples of which are described herein above.

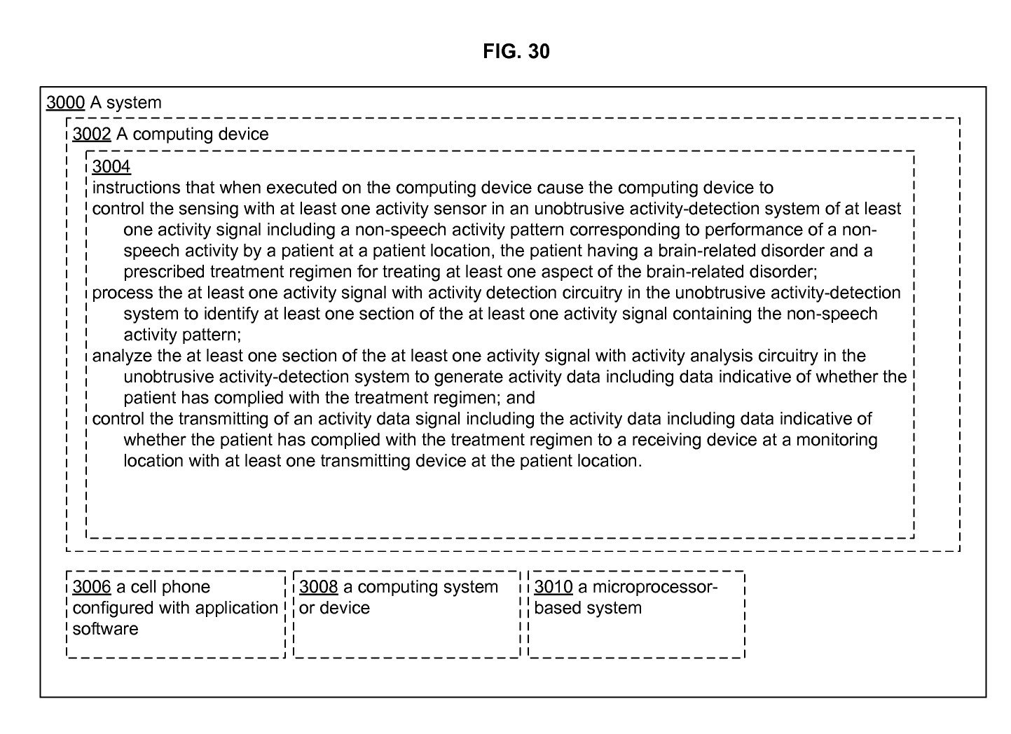

FIG. 30 is a block diagram of a system 3000 for implementing a method as described in connection with FIG. 15. System 3000 includes a computing device 3002 and instructions that when executed on the computing device cause the computing device to control the sensing with at least one activity sensor in an unobtrusive activity-detection system of at least one activity signal including a non-speech activity pattern corresponding to performance of a non-speech activity by a patient at a patient location, the patient having a brain-related disorder and a prescribed treatment regimen for treating at least one aspect of the brain-related disorder; process the at least one activity signal with activity detection circuitry in the unobtrusive activity-detection system to identify at least one section of the at least one activity signal containing the non-speech activity pattern; analyze the at least one section of the at least one activity signal with activity analysis circuitry in the unobtrusive activity-detection system to generate activity data including data indicative of whether the patient has complied with the treatment regimen; and control the transmitting an activity data signal including the activity data including data indicative of whether the patient has complied with the treatment regimen to a receiving device at a monitoring location with at least one transmitting device at the patient location, as indicated at 3004. System 3000 may be, for example, a cell phone configured with application software 3006, a computing system or device 3008, or a microprocessor-based system 3010 or various other systems as described herein. Furthermore, the system may include sensors, input devices, and output devices, e.g., as depicted FIGS. 2, 5, and 7 for example.

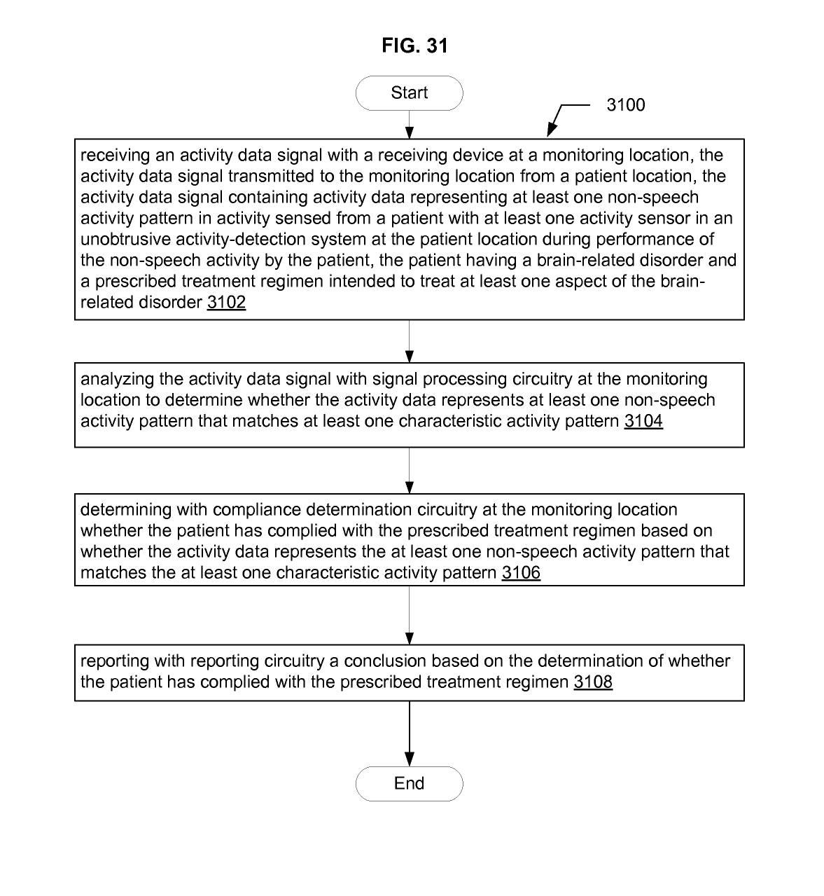

FIG. 31 is a flow diagram of a method 3100 relating to monitoring compliance of a patient with a prescribed treatment regimen. Method 3100 includes receiving an activity data signal with a receiving device at a monitoring location, the activity data signal transmitted to the monitoring location from a patient location, the activity data signal containing activity data representing at least one non-speech activity pattern in activity sensed from a patient with at least one activity sensor in an unobtrusive activity-detection system at the patient location during performance of the non-speech activity by the patient, the patient having a brain-related disorder and a prescribed treatment regimen intended to treat at least one aspect of the brain-related disorder, as indicated at 3102; analyzing the activity data signal with signal processing circuitry at the monitoring location to determine whether the activity data represents at least one non-speech activity pattern that matches at least one characteristic activity pattern, as indicated at 3104; determining with compliance determination circuitry at the monitoring location whether the patient has complied with the prescribed treatment regimen based on whether the activity data represents the at least one non-speech activity pattern that matches the at least one characteristic activity pattern, as indicated at 3106; and reporting with reporting circuitry a conclusion based on the determination of whether the patient has complied with the prescribed treatment regimen, as indicated at 3108. In various aspects, method 3100 is carried out with monitoring system 118 as depicted in FIGS. 1 and 4, for example.

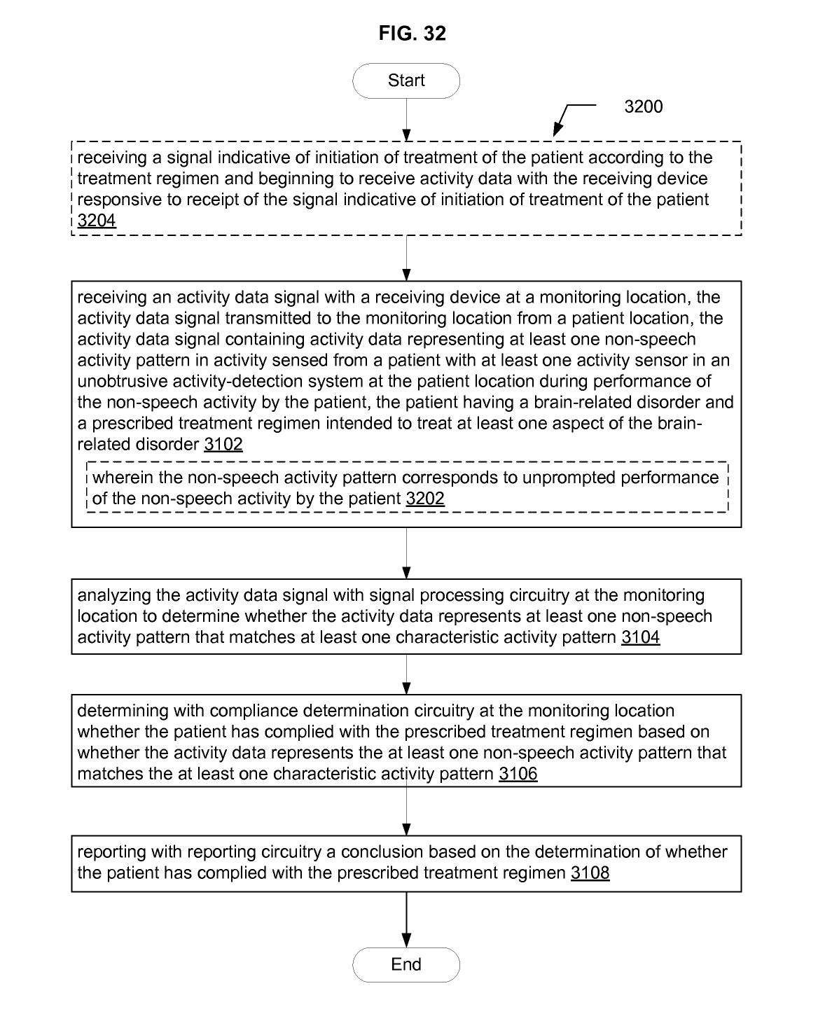

FIGS. 32-48 depict variations and expansions of method 3100 as shown in FIG. 31. In the methods depicted in FIGS. 32-48, steps 3102-3108 are as described generally in connection with FIG. 31. Here and elsewhere, method steps outlined with dashed lines represent steps that are included in some, but not all method aspects, and combinations of steps other than those specifically depicted in the figures are possible as would be known by those having ordinary skill in the relevant art.

FIG. 32 depicts a method 3200, wherein the non-speech activity pattern corresponds to unprompted performance of the non-speech activity by the patient, as indicated at 3202. In an aspect, method 3200 includes receiving a signal indicative of initiation of treatment of the patient according to the treatment regimen and beginning to receive activity data with the receiving device responsive to receipt of the signal indicative of initiation of treatment of the patient, as indicated at 3204.

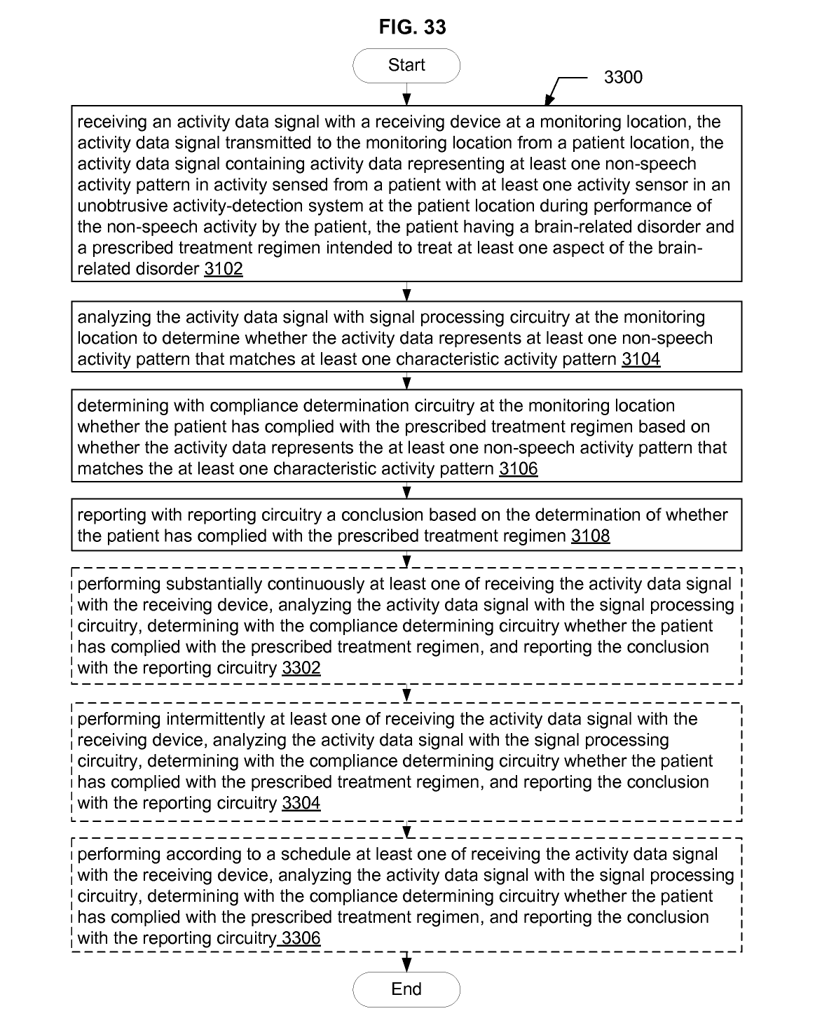

FIG. 33 depicts a method 3300. In an aspect, method 3300 includes performing substantially continuously at least one of receiving the activity data signal with the receiving device, analyzing the activity data signal with the signal processing circuitry, determining with the compliance determining circuitry whether the patient has complied with the prescribed treatment regimen, and reporting the conclusion with the reporting circuitry, as indicated at 3302. In another aspect, method 3300 includes performing intermittently at least one of receiving the activity data signal with the receiving device, analyzing the activity data signal with the signal processing circuitry, determining with the compliance determining circuitry whether the patient has complied with the prescribed treatment regimen, and reporting the conclusion with the reporting circuitry, as indicated at 3304. In another aspect, method 3300 includes performing according to a schedule at least one of receiving the activity data signal with the receiving device, analyzing the activity data signal with the signal processing circuitry, determining with the compliance determining circuitry whether the patient has complied with the prescribed treatment regimen, and reporting the conclusion with the reporting circuitry, as indicated at 3306.

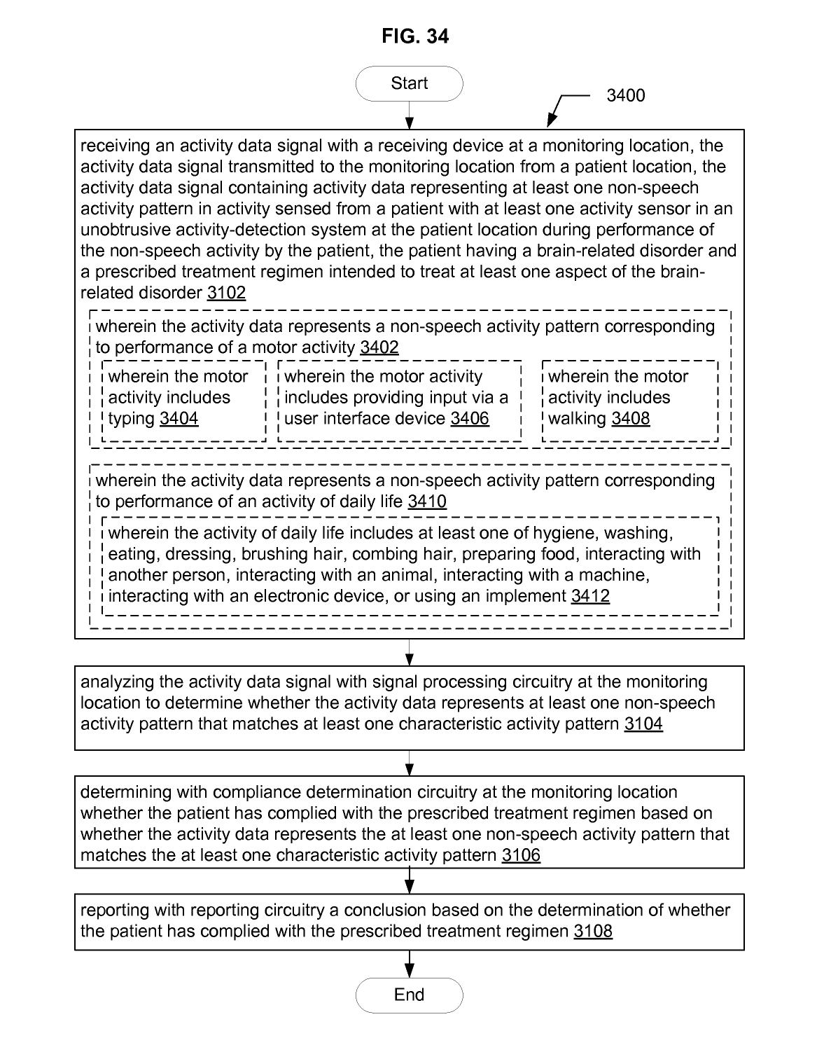

Aspects of a method 3400 are shown in FIG. 34. In one aspect, the activity data represents a non-speech activity pattern corresponding to performance of a motor activity, as indicated at 3402, which in various aspects includes typing, as indicated at 3404; providing input via a user interface device, as indicated at 3406; or walking, as indicated at 3408.

In another aspect, the activity data represents a non-speech activity pattern corresponding to performance of an activity of daily life, as indicated at 3410. For example, in various aspects the activity of daily life includes at least one of hygiene, washing, eating, dressing, brushing hair, combing hair, preparing food, interacting with another person, interacting with an animal, interacting with a machine, interacting with an electronic device, or using an implement, as indicated at 3412.

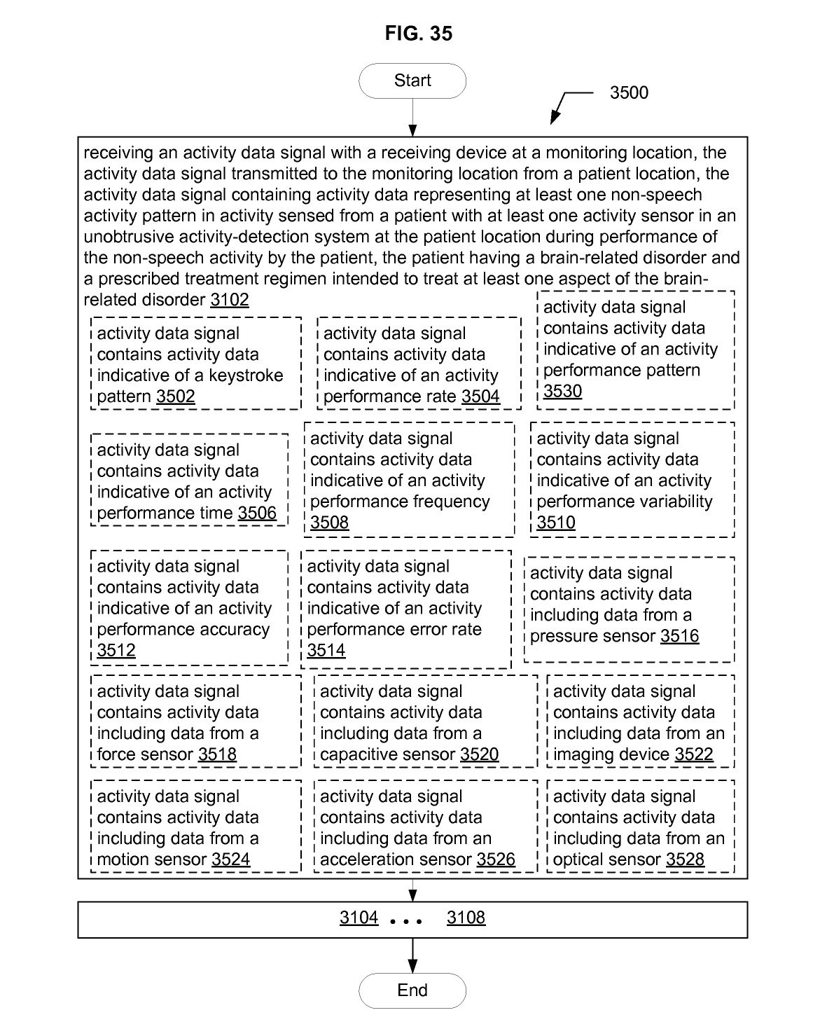

Further aspects relating to receipt of the activity data signal are shown in method 3500 depicted in FIG. 35. In various aspects, the activity data signal contains activity data indicative of a keystroke pattern, as indicated at 3502; activity data indicative of an activity performance pattern, as indicated at 3530; activity data indicative of an activity performance rate, as indicated at 3504; activity data indicative of an activity performance time, as indicated at 3506; activity data indicative of an activity performance frequency, as indicated at 3508; activity data indicative of an activity performance variability, as indicated at 3510; activity data indicative of an activity performance accuracy, as indicated at 3512; activity data indicative of an activity performance error rate, as indicated at 3514; activity data including data from a pressure sensor, as indicated at 3516; activity data including data from a force sensor, as indicated at 3518; activity data including data from a capacitive sensor, as indicated at 3520; activity data including data from an imaging device, as indicated at 3522; activity data including data from a motion sensor, as indicated at 3524; activity data including data from an acceleration sensor, as indicated at 3526; and activity data including data from an optical sensor, as indicated at 3528.

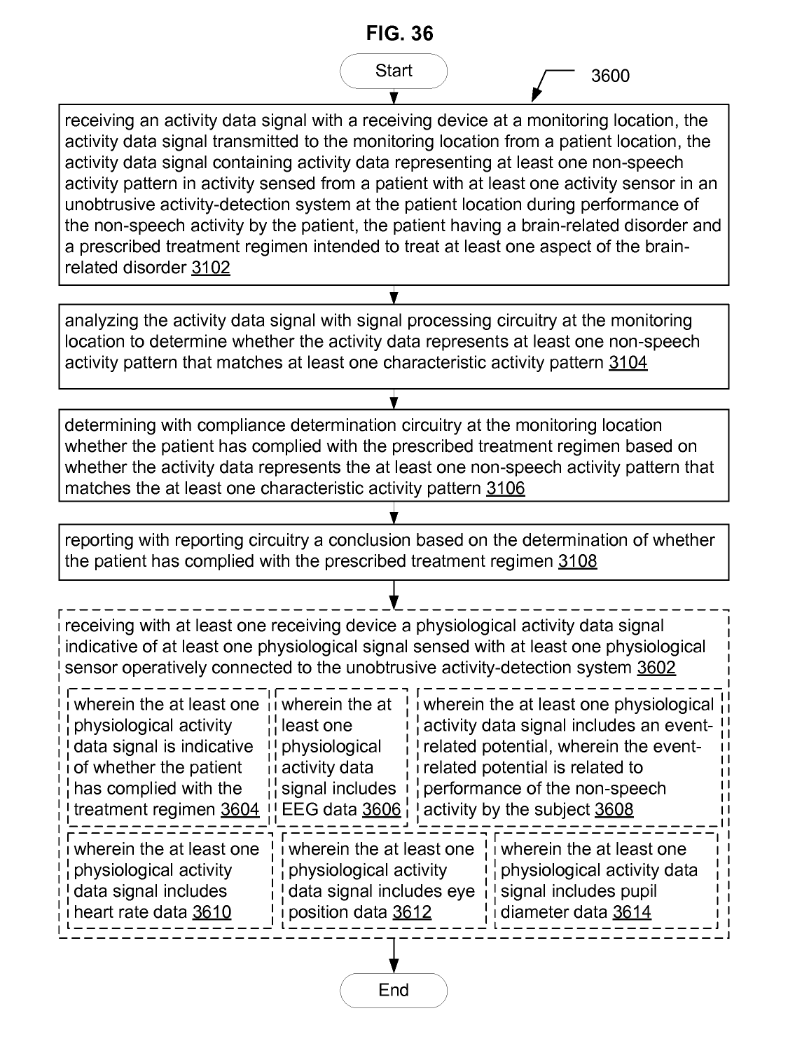

FIG. 36 depicts aspects of a method 3600, which includes receiving with at least one receiving device a physiological activity data signal indicative of at least one physiological signal sensed with at least one physiological sensor operatively connected to the unobtrusive activity-detection system, as indicated at 3602. In an aspect, the at least one physiological activity data signal is indicative of whether the patient has complied with the treatment regimen, as indicated at 3604. In various aspects, the at least one physiological activity data signal includes EEG data, as indicated at 3606; an event-related potential, wherein the event-related potential is related to performance of the non-speech activity by the subject, as indicated at 3608; heart rate data, as indicated at 3610; eye position data, as indicated at 3612; or pupil diameter data, as indicated at 3614.

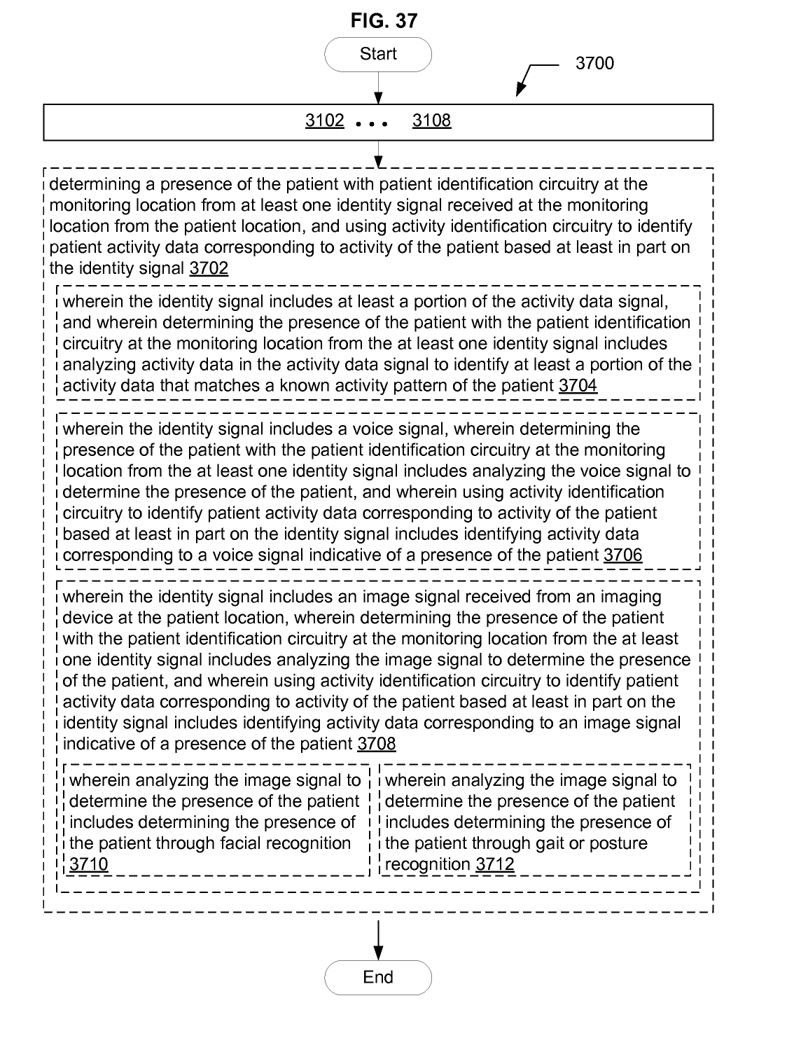

FIG. 37 depicts aspect of method 3700, which includes determining a presence of the patient with patient identification circuitry at the monitoring location from at least one identity signal received at the monitoring location from the patient location, and using activity identification circuitry to identify patient activity data corresponding to activity of the patient based at least in part on the identity signal, as indicated at 3702. In an aspect, the identity signal includes at least a portion of the activity data signal, and wherein determining the presence of the patient with the patient identification circuitry at the monitoring location from the at least one identity signal includes analyzing activity data in the activity data signal to identify at least a portion of the activity data that matches a known activity pattern of the patient, as indicated at 3704. In an aspect, the identity signal includes a voice signal, wherein determining the presence of the patient with the patient identification circuitry at the monitoring location from the at least one identity signal includes analyzing the voice signal to determine the presence of the patient, and wherein using activity identification circuitry to identify patient activity data corresponding to activity of the patient based at least in part on the identity signal includes identifying activity data corresponding to a voice signal indicative of a presence of the patient, as indicated at 3706. In an aspect, the identity signal includes an image signal received from an imaging device at the patient location, wherein determining the presence of the patient with the patient identification circuitry at the monitoring location from the at least one identity signal includes analyzing the image signal to determine the presence of the patient, and wherein using activity identification circuitry to identify patient activity data corresponding to activity of the patient based at least in part on the identity signal includes identifying activity data corresponding to an image signal indicative of a presence of the patient, as indicated at 3708. For example, in an aspect, analyzing the image signal to determine the presence of the patient includes determining the presence of the patient through facial recognition, as indicated at 3710. In another aspect, analyzing the image signal to determine the presence of the patient includes determining the presence of the patient through gait or posture recognition, as indicated at 3712.

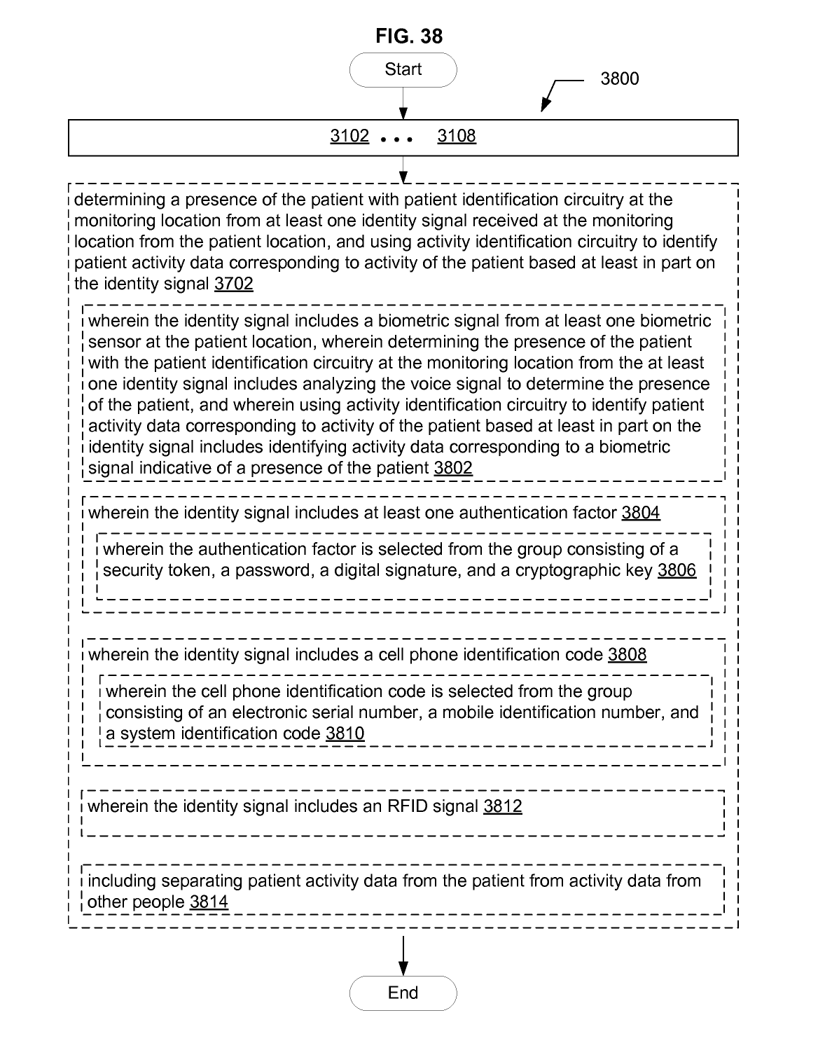

FIG. 38 depicts a method 3800, showing further aspects relating to determination of the presence of the patient with patient identification circuitry at 3702, which is as described in connection with FIG. 37. As indicated at 3802, in an aspect the identity signal includes a biometric signal from at least one biometric sensor at the patient location, wherein determining the presence of the patient with the patient identification circuitry at the monitoring location from the at least one identity signal includes analyzing the voice signal to determine the presence of the patient, and wherein using activity identification circuitry to identify patient activity data corresponding to activity of the patient based at least in part on the identity signal includes identifying activity data corresponding to a biometric signal indicative of a presence of the patient.

In another aspect, the identity signal includes at least one authentication factor, as indicated at 3804. For example, in various aspects the authentication factor is selected from the group consisting of a security token, a password, a digital signature, and a cryptographic key, as indicated at 3806. In another aspect, the identity signal includes a cell phone identification code, as indicated at 3808, for example, an electronic serial number, a mobile identification number, and a system identification code, as indicated at 3810. In another aspect, the identity signal includes an RFID signal, as indicated at 3812. In yet another aspect, method 3800 includes separating patient activity data from the patient from activity data from other people, as indicated at 3814.

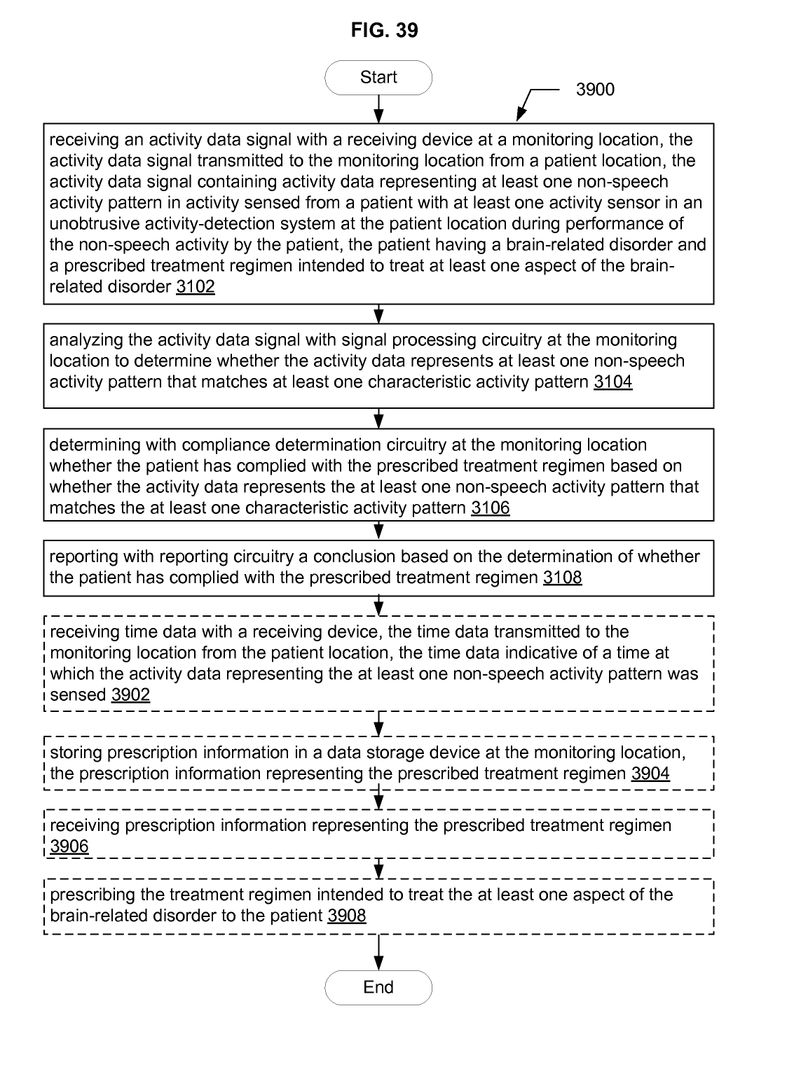

FIG. 39 depicts method 3900, which includes, in various aspects, receiving time data with a receiving device, the time data transmitted to the monitoring location from the patient location, the time data indicative of a time at which the activity data representing the at least one non-speech activity pattern was sensed, as indicated at 3902; storing prescription information in a data storage device at the monitoring location, the prescription information representing the prescribed treatment regimen, as indicated at 3904; receiving prescription information representing the prescribed treatment regimen, as indicated at 3906; prescribing the treatment regimen intended to treat the at least one aspect of the brain-related disorder to the patient, as indicated at 3908.

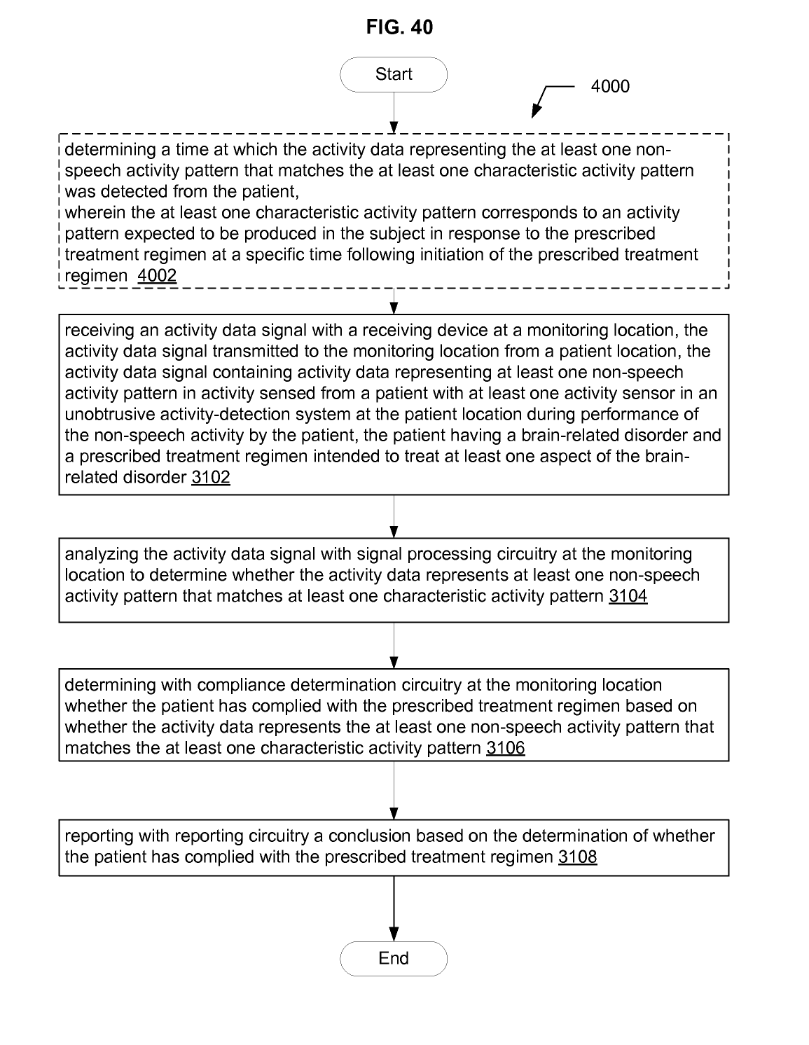

FIG. 40 depicts a method 4000, which includes determining a time at which the activity data representing the at least one non-speech activity pattern that matches the at least one characteristic activity pattern was detected from the patient, wherein the at least one characteristic activity pattern corresponds to an activity pattern expected to be produced in the subject in response to the prescribed treatment regimen at a specific time following initiation of the prescribed treatment regimen, as indicated 4002.

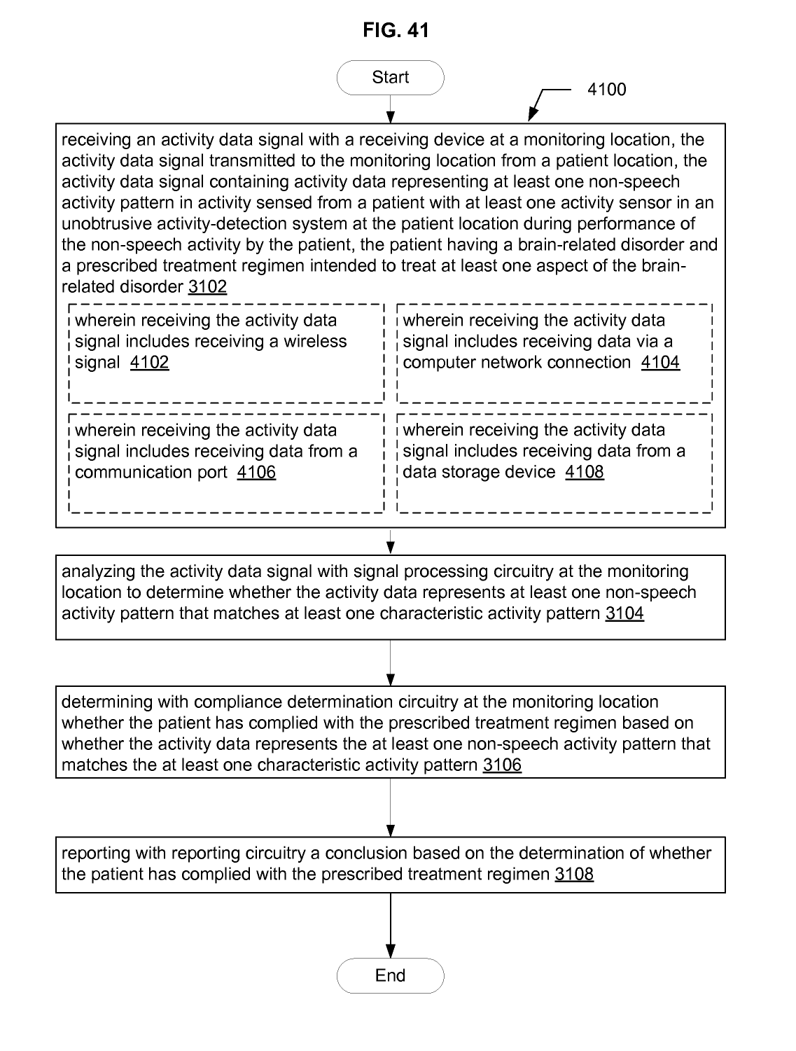

FIG. 41 depicts method 4100 illustrating further aspects relating to receiving an activity data signal at 3102. In various aspects of method 4100, receiving the activity data signal includes at least one of receiving a wireless signal, as indicated at 4102; receiving data via a computer network connection, as indicated at 4104; receiving data from a communication port, as indicated at 4106; and receiving data from a data storage device, as indicated at 4108.

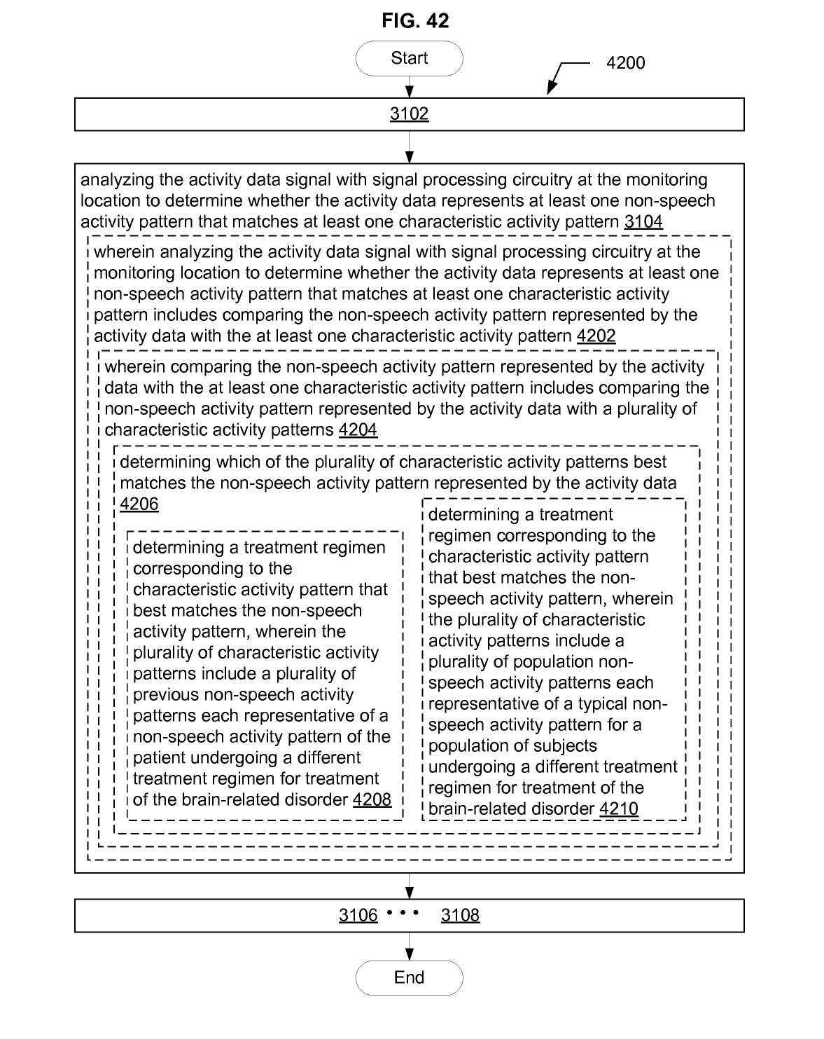

FIG. 42 depicts method 4200, illustrating further aspects relating to analyzing the activity data signal at 3104. In an aspect, analyzing the activity data signal with signal processing circuitry at the monitoring location to determine whether the activity data represents at least one non-speech activity pattern that matches at least one characteristic activity pattern includes comparing the non-speech activity pattern represented by the activity data with the at least one characteristic activity pattern, as indicated at 4202. In an aspect, comparing the non-speech activity pattern represented by the activity data with the at least one characteristic activity pattern includes comparing the non-speech activity pattern represented by the activity data with a plurality of characteristic activity patterns, as indicated at 4204. In connection therewith, method 4200 includes determining which of the plurality of characteristic activity patterns best matches the non-speech activity pattern represented by the activity data, as indicated at 4206. For example, in an aspect method 4200 includes determining a treatment regimen corresponding to the characteristic activity pattern that best matches the non-speech activity pattern, wherein the plurality of characteristic activity patterns include a plurality of previous non-speech activity patterns each representative of a non-speech activity pattern of the patient undergoing a different treatment regimen for treatment of the brain-related disorder, as indicated at 4208. In another aspect, method 4200 includes determining a treatment regimen corresponding to the characteristic activity pattern that best matches the non-speech activity pattern, wherein the plurality of characteristic activity patterns include a plurality of population non-speech activity patterns each representative of a typical non-speech activity pattern for a population of subjects undergoing a different treatment regimen for treatment of the brain-related disorder, as indicated at 4210.

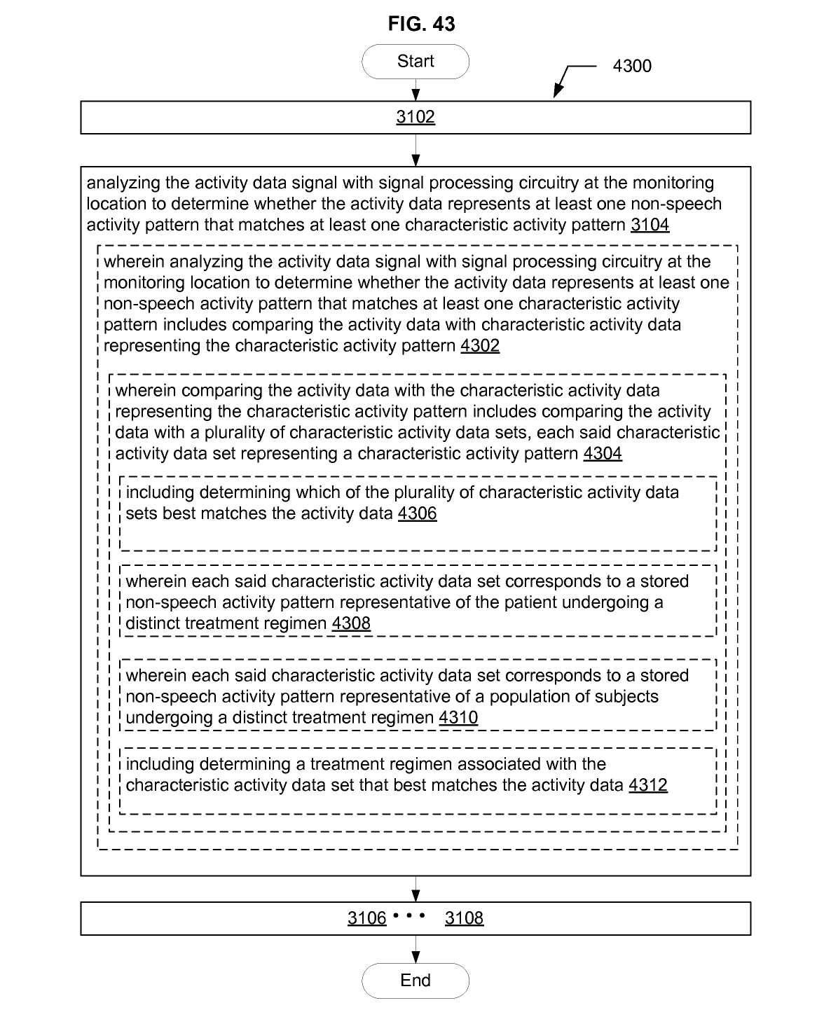

FIG. 43 depicts a method 4300, wherein analyzing the activity data signal with signal processing circuitry at the monitoring location to determine whether the activity data represents at least one non-speech activity pattern that matches at least one characteristic activity pattern includes comparing the activity data with characteristic activity data representing the characteristic activity pattern, as indicated at 4302. In an aspect, comparing the activity data with the characteristic activity data representing the characteristic activity pattern includes comparing the activity data with a plurality of characteristic activity data sets, each said characteristic activity data set representing a characteristic activity pattern, as indicated at 4304. The method may also include determining which of the plurality of characteristic activity data sets best matches the activity data, as indicated at 4306. In an aspect, each said characteristic activity data set corresponds to a stored non-speech activity pattern representative of the patient undergoing a distinct treatment regimen, as indicated at 4308. In an aspect, each said characteristic activity data set corresponds to a stored non-speech activity pattern representative of a population of subjects undergoing a distinct treatment regimen, as indicated at 4310. The method may include determining a treatment regimen associated with the characteristic activity data set that best matches the activity data, as indicated at 4312.

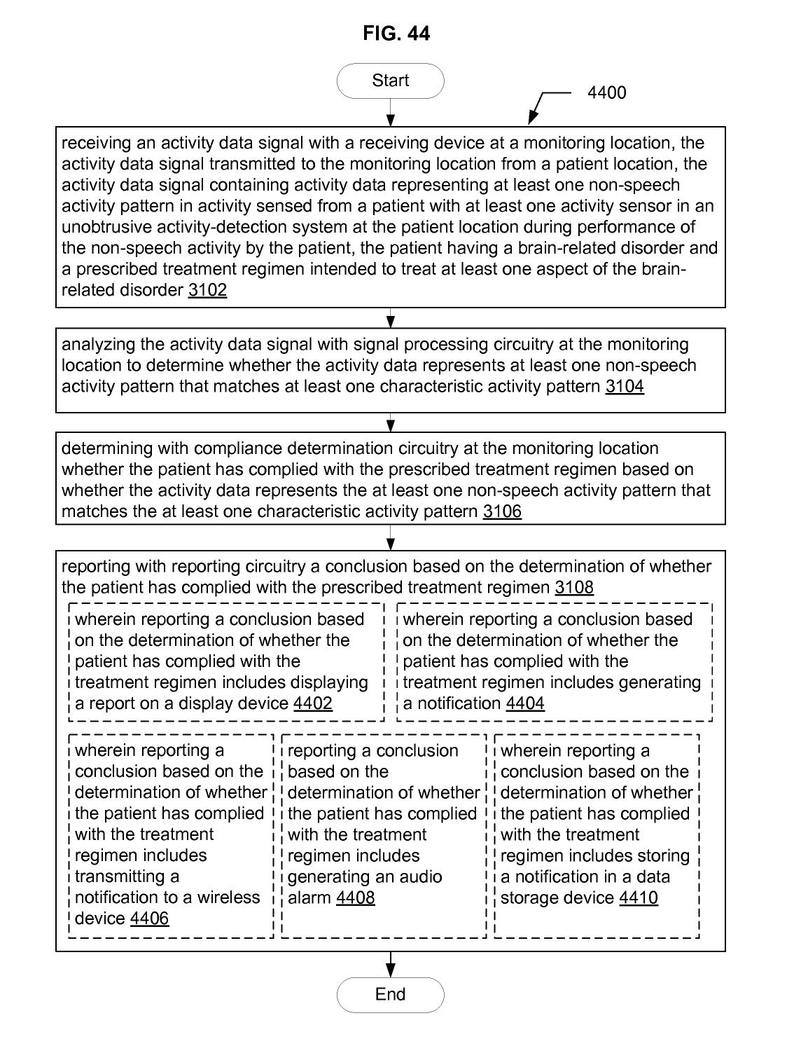

FIG. 44 depicts aspects of a method 4400 relating to reporting with reporting circuitry a conclusion based on the determination of whether the patient has complied with the prescribed treatment regimen, as shown at 3108. In an aspect, reporting a conclusion based on the determination of whether the patient has complied with the treatment regimen includes displaying a report on a display device, as indicated at 4402.

In another aspect, reporting a conclusion based on the determination of whether the patient has complied with the treatment regimen includes generating a notification, as indicated at 4404. In other aspects, reporting a conclusion based on the determination of whether the patient has complied with the treatment regimen includes one or more of transmitting a notification to a wireless device, as indicated at 4406; generating an audio alarm, as indicated at 4408; or storing a notification in a data storage device, as indicated at 4410.

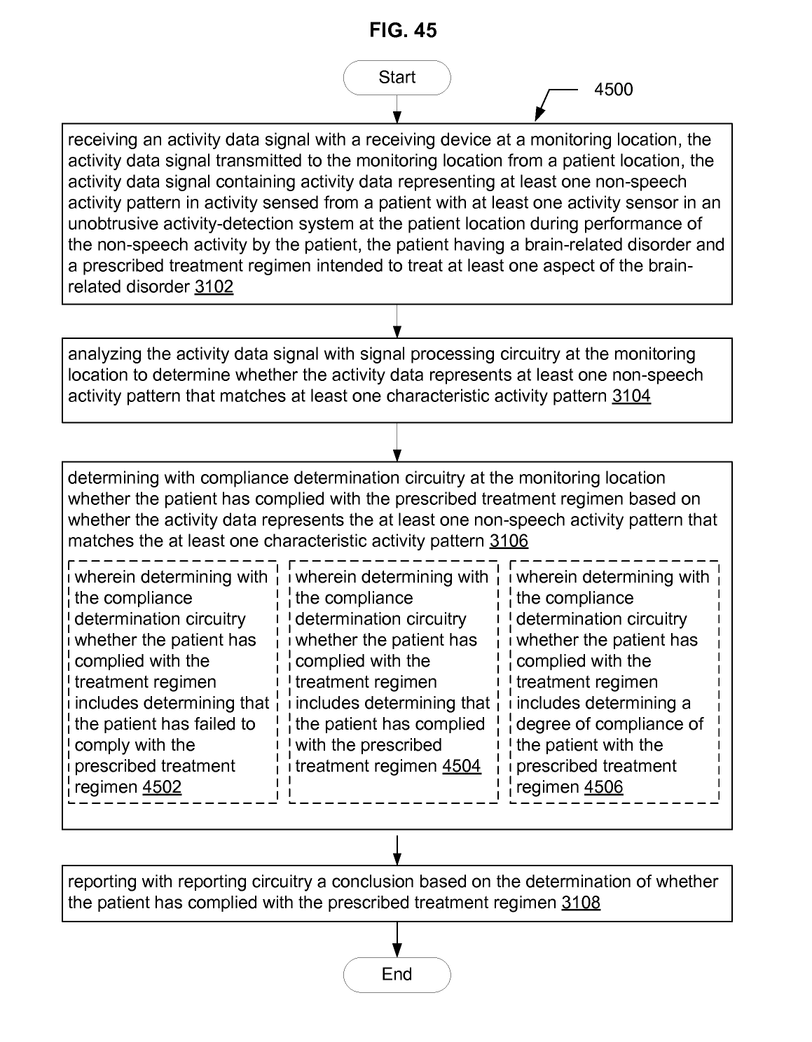

FIG. 45 depicts method 4500, showing method aspects relating to determining whether the patient has complied with the prescribed treatment regimen, at 3106. In an aspect, determining with the compliance determination circuitry whether the patient has complied with the treatment regimen includes determining that the patient has failed to comply with the prescribed treatment regimen, as indicated at 4502. In another aspect, wherein determining with the compliance determination circuitry whether the patient has complied with the treatment regimen includes determining that the patient has complied with the prescribed treatment regimen, as indicated at 4504. In another aspect, determining with the compliance determination circuitry whether the patient has complied with the treatment regimen includes determining a degree of compliance of the patient with the prescribed treatment regimen, as indicated at 4506.

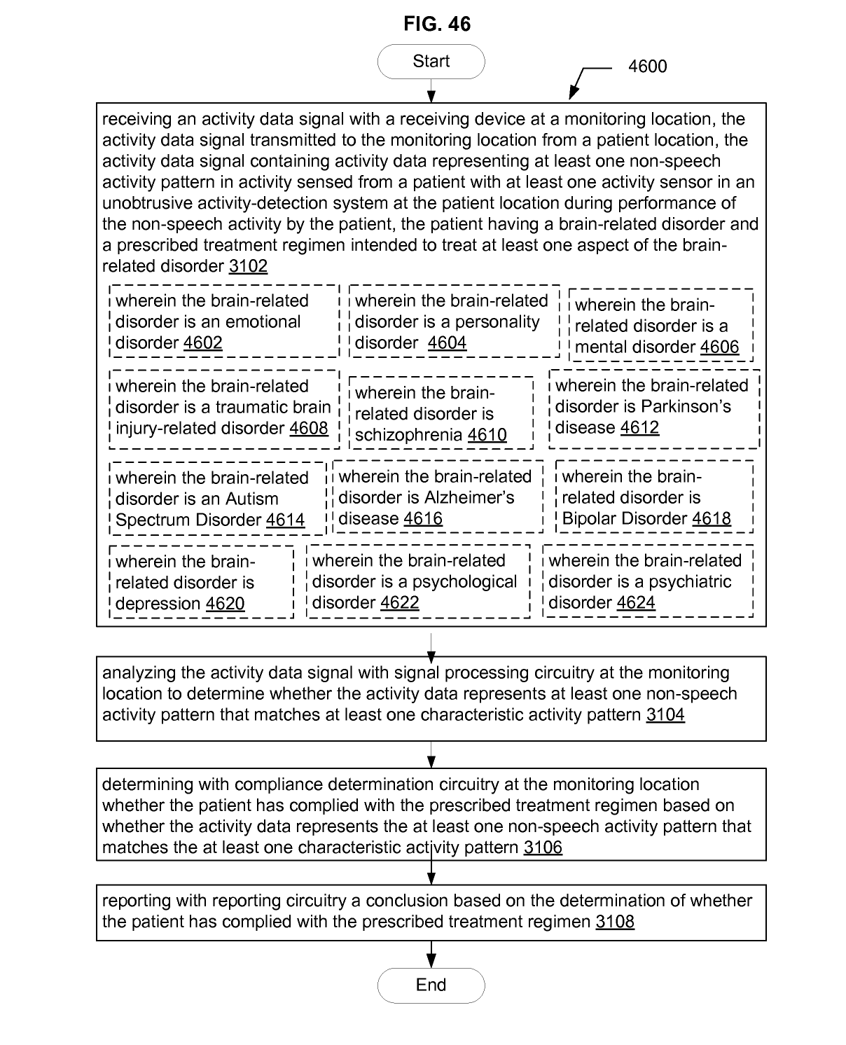

FIG. 46 depicts a method 4600, in which, in various aspects, the brain-related disorder is an emotional disorder, as indicated at 4602; a personality disorder, as indicated at 4604; a mental disorder, as indicated at 4606; a traumatic brain injury-related disorder, as indicated at 4608; schizophrenia, as indicated at 4610; Parkinson's disease, as indicated at 4612; an Autism Spectrum Disorder, as indicated at 4614; Alzheimer's disease, as indicated at 4616; Biopolar Disorder, as indicated at 4618; depression, as indicated at 4620; a psychological disorder, as indicated at 4622; or a psychiatric disorder, as indicated at 4624.

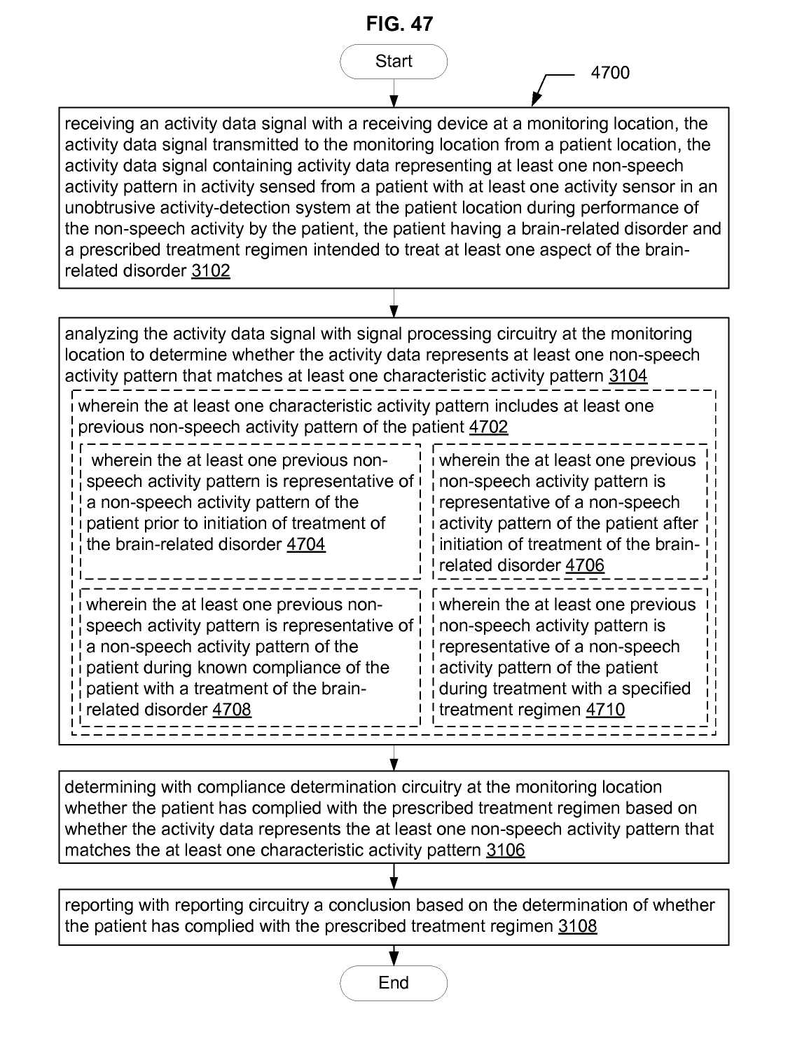

FIG. 47 depicts a method 4700, wherein the at least one characteristic activity pattern includes at least one previous non-speech activity pattern of the patient, as indicated at 4702. In various aspects, the at least one previous non-speech activity pattern is representative of a non-speech activity pattern of the patient prior to initiation of treatment of the brain-related disorder, as indicated at 4704; a non-speech activity pattern of the patient after initiation of treatment of the brain-related disorder, as indicated at 4706; a non-speech activity pattern of the patient during known compliance of the patient with a treatment of the brain-related disorder, as indicated at 4708; or a non-speech activity pattern of the patient during treatment with a specified treatment regimen, as indicated at 4710.

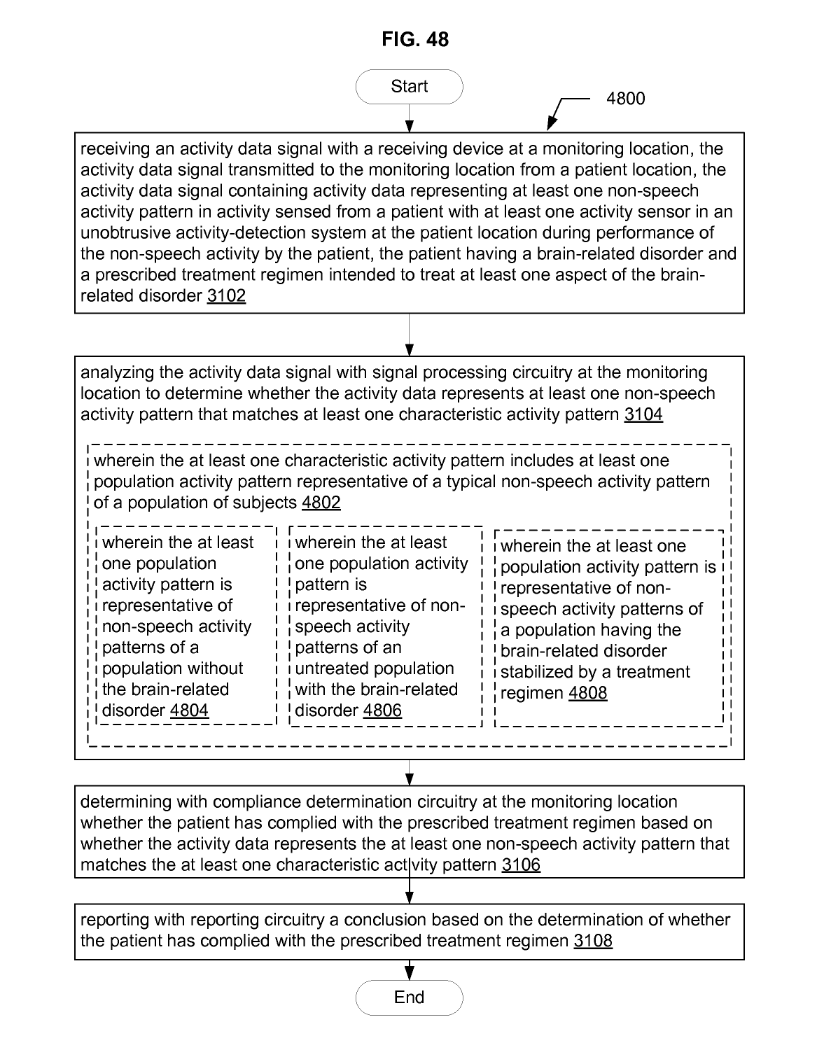

FIG. 48 depicts a method 4800, wherein the at least one characteristic activity pattern includes at least one population activity pattern representative of a typical non-speech activity pattern of a population of subjects, as indicated at 4802. In various aspects, the at least one population activity pattern is representative of non-speech activity patterns of a population without the brain-related disorder, as indicated at 4804; an untreated population with the brain-related disorder, as indicated at 4806; or a population having the brain-related disorder stabilized by a treatment regimen, as indicated at 4808.

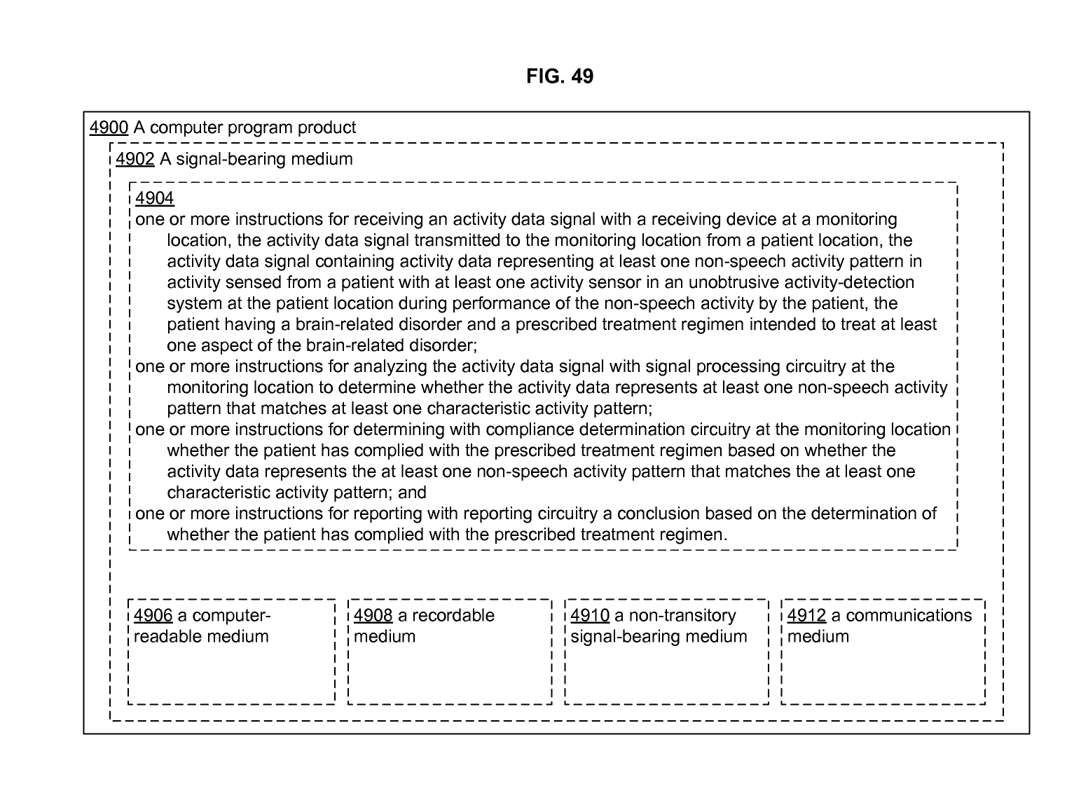

FIG. 49 is a block diagram of a computer program product 4900 for implementing a method as described in connection with FIG. 31. Computer program product 4900 includes a signal-bearing medium 4902 bearing one or more instructions for receiving an activity data signal with a receiving device at a monitoring location, the activity data signal transmitted to the monitoring location from a patient location, the activity data signal containing activity data representing at least one non-speech activity pattern in activity sensed from a patient with at least one activity sensor in an unobtrusive activity-detection system at the patient location during performance of the non-speech activity by the patient, the patient having a brain-related disorder and a prescribed treatment regimen intended to treat at least one aspect of the brain-related disorder, one or more instructions for analyzing the activity data signal with signal processing circuitry at the monitoring location to determine whether the activity data represents at least one non-speech activity pattern that matches at least one characteristic activity pattern, one or more instructions for determining with compliance determination circuitry at the monitoring location whether the patient has complied with the prescribed treatment regimen based on whether the activity data represents the at least one non-speech activity pattern that matches the at least one characteristic activity pattern, and one or more instructions for reporting with reporting circuitry a conclusion based on the determination of whether the patient has complied with the prescribed treatment regimen, as indicated at 4904. Signal-bearing medium 4902 may be, for example, a computer-readable medium 4906, a recordable medium 4908, a non-transitory signal-bearing medium 4910, or a communications medium 4912, examples of which are described herein above.

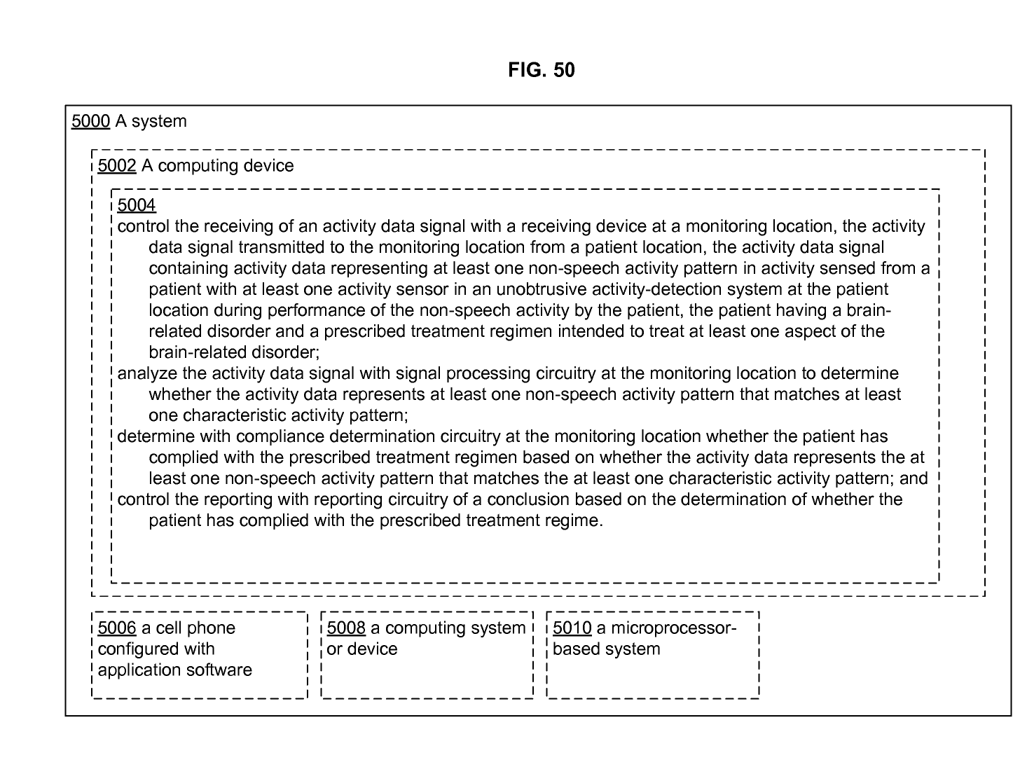

FIG. 50 is a block diagram of a system 5000 for implementing a method as described in connection with FIG. 31. System 5000 includes a computing device 5002 and instructions that when executed on the computing device cause the computing device to control the receiving of an activity data signal with a receiving device at a monitoring location, the activity data signal transmitted to the monitoring location from a patient location, the activity data signal containing activity data representing at least one non-speech activity pattern in activity sensed from a patient with at least one activity sensor in an unobtrusive activity-detection system at the patient location during performance of the non-speech activity by the patient, the patient having a brain-related disorder and a prescribed treatment regimen intended to treat at least one aspect of the brain-related disorder; analyze the activity data signal with signal processing circuitry at the monitoring location to determine whether the activity data represents at least one non-speech activity pattern that matches at least one characteristic activity pattern; determine with compliance determination circuitry at the monitoring location whether the patient has complied with the prescribed treatment regimen based on whether the activity data represents the at least one non-speech activity pattern that matches the at least one characteristic activity pattern; and control the reporting with reporting circuitry of a conclusion based on the determination of whether the patient has complied with the prescribed treatment regimen, as indicated at 5004. System 5000 may be, for example, a cell phone configured with application software 5006, a computing system or device 5008, or a microprocessor-based system 5010.

In other aspects, systems may be constructed which utilizes two or more activity signals detected from the patient in order to determine whether the patient has complied with a prescribed treatment regimen. Such systems may utilize various combinations of activity signals as described herein, or utilize one or more activity signals as described herein in combination with an audio signal including speech from the patient. Information regarding compliance with a treatment regimen can be based in part upon analysis of patient speech.

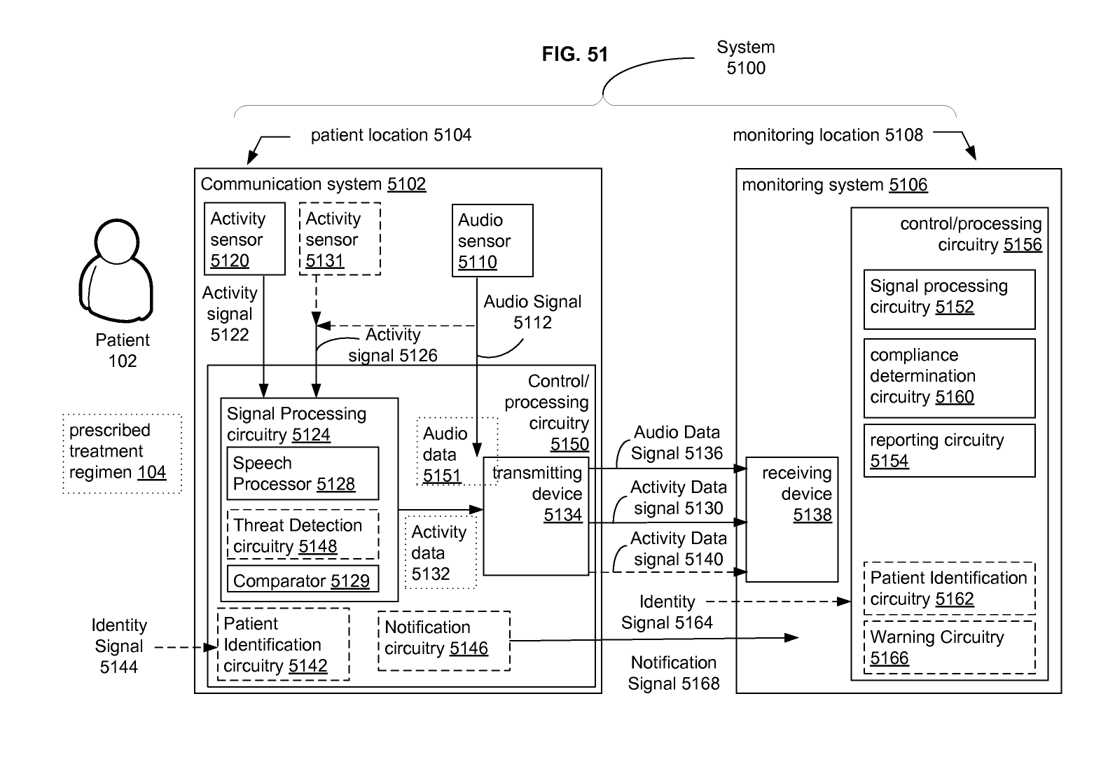

FIG. 51 is a block diagram of a system 5100 for monitoring compliance of a patient with a treatment regimen based upon two or more sensed signals. System 5100 includes communication system 5102 at patient location 5104 and monitoring system 5106 at monitoring location 5108. In general, communication system 5102 includes components shown in unobtrusive activity detection system 108 in FIG. 2, as well as any additional components required for perform communication system functions. Communication system 5102 includes at least one audio sensor 5110 for sensing at least one audio signal 5112, which includes patient speech from patient 102 at a patient location 5104 during use of communication system 5102. In an aspect, communication system 5102 includes a telephone (e.g., as depicted in FIG. 7), an intercommunication system (e.g., as depicted in FIG. 13), or a radio communication system, and audio sensor is a microphone or other audio sensing device as known by those of ordinary skill in the art. Patient 102 has a brain-related disorder and a prescribed treatment regimen 104 for treating at least one aspect of the brain-related disorder. Communication system 5102 includes at least one first activity sensor 5120 for sensing at least one first activity signal 5122 indicative of a first activity of the patient. Communication system 5102 includes signal processing circuitry 5124, which is configured to process the at least one first activity signal 5122 and at least one second activity signal 5126, which indicative of a second activity of the patient, to generate at least one activity data signal 5130, the activity data signal 5130 containing activity data 5132 indicative of whether the patient has complied with the treatment regimen. Communication system 5102 also includes at least one transmitting device 5134 at the patient location for transmitting the at least one activity data signal 5130 and at least one audio data signal 5136 based on the at least one audio signal 5112 to a receiving device 5138 at monitoring location 5108. In an aspect, activity signal 5126 includes audio signal 5112 from audio sensor 5110, which can supply information regarding speech or vocal activity of patient 102. In an aspect, signal processing circuitry 5124 includes speech processor 5128. In an aspect, speech processor 5128 is configured to process the at least one audio signal 5112 to identify at least one portion of the at least one audio signal 5112 containing spontaneous speech of the patient. In an aspect, speech processor 5128 is configured to process at least one audio signal 5112 to exclude at least one portion of at least one audio signal 5112 that does not contain spontaneous speech of the patient. In an aspect, activity data 5132 includes the at least one section of the at least one audio signal 5112 containing spontaneous speech of the patient.

In an aspect, speech processor 5128 is configured to process at least one audio signal 5112 to determine at least one speech pattern of the patient. In an aspect, activity data 5132 includes the at least one speech pattern. A speech pattern can be defined as a consistent, characteristic form, style, or method of speech comprising a distribution or arrangement of repeated or corresponding parts composed of qualities, acts, or tendencies. In an embodiment a speech pattern can include one or more qualities of diction, elocution, inflection, and/or intonation. In an embodiment a speech pattern can include aspects of language at the lexical level, sentential level, or discourse level. In an embodiment, a speech pattern may conform to the Thought, Language, and Communication Scale and/or Thought and Language Index. Reviews describing speech patterns and linguistic levels and the tools used to study them include Covington M. A., et al. “Schizophrenia and the structure of language: The linguist's view,” Schizophrenia Research 77: 85-98, 2005, and Kuperberg and Caplan (2003 Book Chapter: Language Dysfunction in Schizophrenia), which are both incorporated herein by reference.

In an embodiment, a speech pattern includes a linguistic pattern determined at the lexical level. A speech pattern may include a frequency of, for example, pauses, words, or phrases. For example, a speech pattern may include a frequency of pauses. A higher frequency of pauses or reduced verbal fluency can be indicative of alogia associated with a brain disorder, e.g., bipolar disorder, depression, or schizophrenia. For example, a speech pattern may include a frequency of dysfluencies (“uhs” and “ums”). A higher than average frequency of dysfluencies may indicate a slowed speech, the inability to think clearly, or a deliberate attempt to appear unaffected by illness, all of which have been associated with psychological pathologies. For example, a speech pattern may include a distribution of pauses and dysfluencies. A high frequency and particular distribution of pauses and dysfluencies may be indicative of anomia associated with schizophrenia or with an aphasia due to brain injury. For example, a speech pattern may include a frequency of neologisms and/or word approximations, or glossomania. Higher than average frequencies of neologisms and/or word approximations, or glossomania, have been associated with disorders such as schizophrenia, schizoaffective disorder, or mania. For example, a speech pattern may include a frequency of word production. A frequency of word production lower than the norm may be indicative of a brain disorder such as schizophrenia. An excessive speed during speech, as in pressured speech, may be indicative of a brain disorder such as the mania of bipolar disorder, while reduced speed may be indicative of depression or a depressive episode. For example, a pattern may include a type:token ratio (i.e., number of different words (types) in relation to the total number of words spoken (tokens)). A type:token ratio that is generally lower than the norm can be indicative of schizophrenia. For example, a speech pattern may include a frequency of specific words. Quantitative word counts have been used as a tool in the identification and examination of abnormal psychological processes including major depression, paranoia, and somatization disorder. A high frequency of negative emotion words or death-related words may be indicative of depression. Psychologically relevant words can include those listed in one or more dictionaries of the Linguistic Inquiry and Word Count (LIWC) program (see Tausczik and Pennebaker, “The Psychological Meaning of Words: LIWC and Computerized Text Analysis Methods,” Journal of Language and Social Psychology 29(1): 24-54, 2010, which is incorporated herein by reference). Words interpreted as carrying normative emotional qualities are found in dictionaries of two programs, Affective Norms for English Words (ANEW) and Dictionary of Affect in Language (DAL) (see Whissell C., “A comparison of two lists providing emotional norms for English words (ANEW and the DAL),” Psychol Rep., 102(2):597-600, 2008, which is incorporated herein by reference).

In an embodiment, a speech pattern includes a linguistic pattern determined at the sentential level or discourse level. For example, a speech pattern can include a consistent grammatical style. A pattern comprising a style that is grammatically deviant from the norm might include the overuse of the past tense, indicating detachment from the subject being discussed. A pattern comprising a style that is grammatically deviant from the norm, e.g., as reflected by a higher percentage of simple sentences and, in compound sentences, fewer dependent clauses may be indicative of schizophrenia. For example, a speech pattern may include a ratio of syntactic complexity (number of clauses and proportion of relative:total clauses). An abnormal ratio may indicate a brain disorder. For example, a speech pattern may include a frequency of subordinate clauses. An increase in subordinate clauses has been observed in the speech of psychopaths (see, e.g., Hancock et al., “Hungry like the wolf: A word-pattern analysis of the language of psychopaths,” Legal and Criminological Psychology, 2011; DOI: 10.1111/j.2044-8333.2011.02025.x, which is incorporated herein by reference). For example, a speech pattern may include a relatedness of lexical content such as semantic or sentential priming. A speech pattern of abnormal priming may indicate a brain disorder such as schizophrenia. For example, a speech pattern may include a frequency of one or more use of cohesive ties, e.g., as demonstrated by references, conjunctions, or lexical cohesion. A low frequency of reference ties has been observed in patients suffering from schizophrenia. For example, a speech pattern may include an hierarchical structure within a discourse, e.g., a systematic structure in which propositions branch out from a central proposition. A speech pattern lacking a systematic structure may be indicative of schizophrenia.

For example, a speech pattern including a linguistic pattern determined at the sentential level or discourse level may include a representation of content of thought (what the patient is talking about). For example, a speech pattern may include a representation of form of thought (the way ideas, sentences, and words are put together). A speech pattern containing representations of content or form of thought that differ from those expected (e.g., as determined from population patterns) may indicate a psychological disorder such as schizophrenia. Examples of representations of content or form of thought observed in schizophrenia include derailment, loss of goal, perseveration, and tangentiality. For example, a speech pattern may include aspects of linguistic pragmatics (e.g., cohesion or coherence). Abnormal patterns in pragmatics may be indicative of a brain disorder such as schizophrenia or mania. Examples of speech patterns and content of thought are discussed by Covington, et al., idem, and by Kuperberg and Caplan idem. A program for classifying parts of speech (e.g., noun, verb, adjective, etc.) based on the surrounding context and analysis of semantic content has been developed and is available under the Wmatrix interface (http://ucrel.lancs.ac.uk/wmatrix/) and has been used to analyze the speech of psychopaths (see Hancock, idem).

In an embodiment, a speech pattern includes an acoustic quality. In an embodiment a speech pattern includes volume. For example, excessive or reduced volume may be indicative of a symptom of a brain disorder. In an embodiment a speech pattern includes prosody (the rhythm, stress, and intonation of speech). For example, aprosody or flattened intonation can be indicative of schizophrenia. In an embodiment, a speech pattern includes a voice quality of phonation. In an embodiment, a speech pattern includes pitch or timbre. For example, abnormalities in pitch have been observed in schizophrenics. For example, a strained quality, choking voice, or creaking voice (laryngealisation) may be indicative of a psychological disorder. Voice qualities and volume in linguistics are discussed by Covington, idem.

For example, the at least one speech pattern may be represented in activity data 5132 in numerical or categorical form. For example, a speech pattern represented in numerical form may include one or more numerical values representing one or more speech parameters. Particular speech parameters represented in a speech pattern may be selected for the purpose of evaluating/monitoring particular brain-related disorders. For example, in an aspect a speech pattern for evaluating/monitoring depression includes values representing the following parameters: speech volume, frequency of word production, frequency of pauses, and frequency of negative value words. In another aspect, a speech pattern for evaluating/monitoring schizophrenia includes values representing frequency of word production, frequency of pauses, frequency of disfluencies, type:token ratio, and speech volume. A speech parameter or pattern may be represented in activity data 5132 in categorical form; for example, frequency of word production may be categorized as low, medium, or high rather than represented by a specific numerical value.

In an aspect, signal processing circuitry 5124 includes a comparator 5129 for comparing speech patterns or parameters of patient 102 with characteristic speech patterns or parameters, in an approach similar to that described above in connection with comparator 254 in FIG. 2, to determine whether the patient has complied with the prescribed treatment regimen. In an aspect, comparator 5129 is configured to compare at least one speech pattern of the patient with a plurality of characteristic speech patterns. In an aspect, the result of such a comparison is either “patient has complied” or “patient has not complied.” In an aspect, signal processing circuitry 5124 is configured to determine that patient 102 has failed to comply with the prescribed treatment regimen. In an aspect, signal processing circuitry 5124 is configured to determine that patient 102 has complied with prescribed treatment regimen 104. Determination of compliance may be accomplished by a thresholding, windowing, or distance computation of one or multiple parameters relative to characteristic threshold or range values for the parameter, and combining results for the multiple parameters. For example, for a given parameter (relating to activity sensed with one or more activity sensor or audio sensor), a patient parameter value higher than a characteristic threshold value may indicate compliance of the patient with the prescribed treatment regimen, while a patient parameter value equal to or lower than the threshold value may indicate non-compliance. As another example, a patient parameter value that lies within a range of characteristic values for the parameter may indicate compliance, while a patient parameter value outside the range of characteristic values indicates non-compliance. Comparator 5129 may utilize various types of distance computations to determine whether patient parameter values are within a threshold distance or distance range from characteristic values. Distance computations based on one or more parameters or data values are known (including, but not limited to, least-squares calculations). Different activity parameters or audio signal parameters may be given different weights depending on how strongly indicative the parameter is of the patient compliance. In an aspect, signal processing circuitry 5124 is configured to determine whether the patient has complied with the prescribed treatment regimen based upon a determination of whether the speech corresponds to at least one of a plurality of characteristic speech patterns. For example, the plurality of characteristic speech patterns can include multiple characteristic speech patterns, each corresponding to a patient speech pattern obtained at a different treatment regimen, for example, different doses of a drug. By identifying which characteristic speech pattern the patient speech pattern matches or is closest to, the drug dose taken by the patient can be determined. For example, the patient may have taken the drug, but at a lesser dose or less often than was prescribed. Accordingly, the patient's speech pattern matches the characteristic speech pattern associated with the lesser dose of drug, indicating partial, but not full, compliance of the patient with the prescribed treatment regimen.

In an aspect, speech processor 5128 is configured to process at least one audio signal 5112 to determine at least one speech parameter indicative of whether the patient has complied with the prescribed treatment regimen. Speech parameters include, but are not limited to, measures of prosody, rhythm, stress, intonation, variance, intensity/volume, pitch, length of phonemic syllabic segments, and length of rising segments, for example. In an aspect, audio data includes at least one speech parameter, which may include, for example, one or more of prosody, rhythm, stress, intonation, variance, intensity/volume, pitch, length of phonemic syllabic segments, and length of rising segments. In an aspect, signal processing circuitry 5124 includes comparator 5129 for comparing at least one speech parameter of the patient with at least one characteristic speech parameter to determine whether the patient has complied with the prescribed treatment regimen. In an aspect, comparator 5129 is configured to compare at least one speech parameter of the patient with a plurality of characteristic speech parameters to determine whether the patient has complied with the prescribed treatment regimen. For example, in an aspect, the result of such a comparison is either “patient has complied” or “patient has not complied.” In an aspect, comparator 5129 determines a level of compliance of the patient with the prescribed treatment regimen. Determination of compliance, non-compliance, or level of compliance may be performed with comparator 5129 using thresholding, windowing, or distance measurements, for example, as described herein above. Similarly, determination of compliance or non-compliance of patient 102 with a prescribed treatment regimen may be accomplished with the use of comparator 5129 using approaches as described herein above.

In an aspect, activity signal 5126 includes a signal from one or more additional activity sensor(s) 5131. In various aspects, first activity sensor 5120 and any additional activity sensor(s) 5131 include any of the various types of activity sensor 116 described herein above, e.g., as in connection with FIG. 3. In an aspect, signal processing circuitry 5124 processes at least one first activity signal 5122 and at least one second activity signal 5126 using signal processing approaches as described herein above (e.g., as described in connection with activity detection circuitry 122/activity analysis circuitry 126 in FIG. 1), to generate activity data 5132, which is included in activity data signal 5130. In some aspects, more than one activity data signal is generated (e.g., activity data signal 5130 and activity signal 5140). In some aspects, activity data from different activity sensors is transmitted in separate activity data signals. In other aspects, activity data from multiple activity sensors is transmitted in a single activity data signal. In an aspect, audio data signal 5136 is a radio frequency signal containing telecommunication data. In some aspects, audio data signal 5136 is combined with activity data signal 5130. In some aspects, communication system 5102 includes patient identification circuitry 5142, which is used to determine the presence of patient 102 based on identity signal 5144, using an approach as described herein above, e.g., in connection with patient identification circuitry 222 in FIG. 2. In some aspects, communication system 5102 includes notification circuitry 5146, which functions in the same manner as notification circuitry 290 in FIG. 2. In an aspect, communication system 5102 includes threat detection circuitry 5148 in signal processing circuitry 5124. Threat detection circuitry 5148 is used for determining, based upon at least one of the at least one first activity signal and the at least one second activity signal, whether the patient poses a threat. Threat can be determined using approaches as described, for example, in U.S. Patent Application 2006/0190419 dated Aug. 24, 2016 to Bunn et al., and U.S. Patent Application 2006/00208556 dated Feb. 9, 2006 to Bunn et al., both of which are incorporated herein by reference. If it is determined that that patient poses a threat, a notification indicative of the threat is generated with notification circuitry 5146, and the notification is delivered to the threatened party via warning circuitry 5166 in monitoring system 5106. Alternatively, or in addition, warning circuitry may be located separately from monitoring system 5106. Signal processing circuitry 5124, patient identification circuitry 5142, notification circuitry 5146, threat detection circuitry 5148, and transmitting device 5134 are components of control/processing circuitry 5150.

Monitoring system 5106 includes at least one receiving device 5138 for use at a monitoring location 5108 for receiving at least one activity data signal 5130 and at least one audio data signal 5136 (and, optionally one or more additional activity data signal 5140) from communication system 5102 and is similar to receiving device 136 in FIGS. 1 and 4. Audio data signal 5136 includes audio data 5151 representing speech from patient 102 sensed with at least one audio sensor 5110 at the patient location 5104 during use of communication system 5102, and transmitted to the monitoring location 5108. Activity data signal 5130 includes activity data 5132 indicative of whether patient 102 has complied with the prescribed treatment regimen 104. Activity data 5132 represents at least one first activity of the patient. Monitoring system 5106 includes signal processing circuitry 5152, which is configured to process the at least one activity data signal 5130 to determine, based upon the at least one first activity of the patient and at least one second activity of the patient, whether the patient has complied with the prescribed treatment regimen, and reporting circuitry 5154 configured to report a conclusion based on the determination of whether the patient has complied with the prescribed treatment regimen. Signal processing circuitry 5152 is substantially similar to signal processing circuitry 150 as discussed in connection with FIGS. 1 and 4. Reporting circuitry 5154 is substantially the same as reporting circuitry 160 as discussed in connection with FIGS. 1 and 4. Signal processing circuitry 5152 and reporting circuitry 5154 are components of control/processing circuitry 5156 in monitoring system 5106. In an aspect, control/processing circuitry 5156 includes compliance determination circuitry 5160, which functions in the same manner as compliance determination circuitry 156 in FIGS. 1 and 4, as discussed herein above. In an aspect control/processing circuitry 5156 includes patient identification circuitry 5162, which determines a presence of patient 102 at patient location 5104 based on identity signal 5164, in the same manner as patient identification circuitry 410 depicted in FIG. 4 and described herein above. In an aspect, control/processing circuitry 5156 includes warning circuitry 5166, which delivers a warning to a threatened party in response to a notification. The notification is received from the patient location, e.g., in the form of notification signal 5168 from transmitting device 5134, as described herein above. Delivering a warning to a threatened party may include, for example, displaying a warning message, playing a recorded warning message, or generating an audible alarm tone. The warning may be delivered in the same general manner as conclusion 162 is reported by reporting circuitry 160, as described herein above, in connection with FIG. 4.

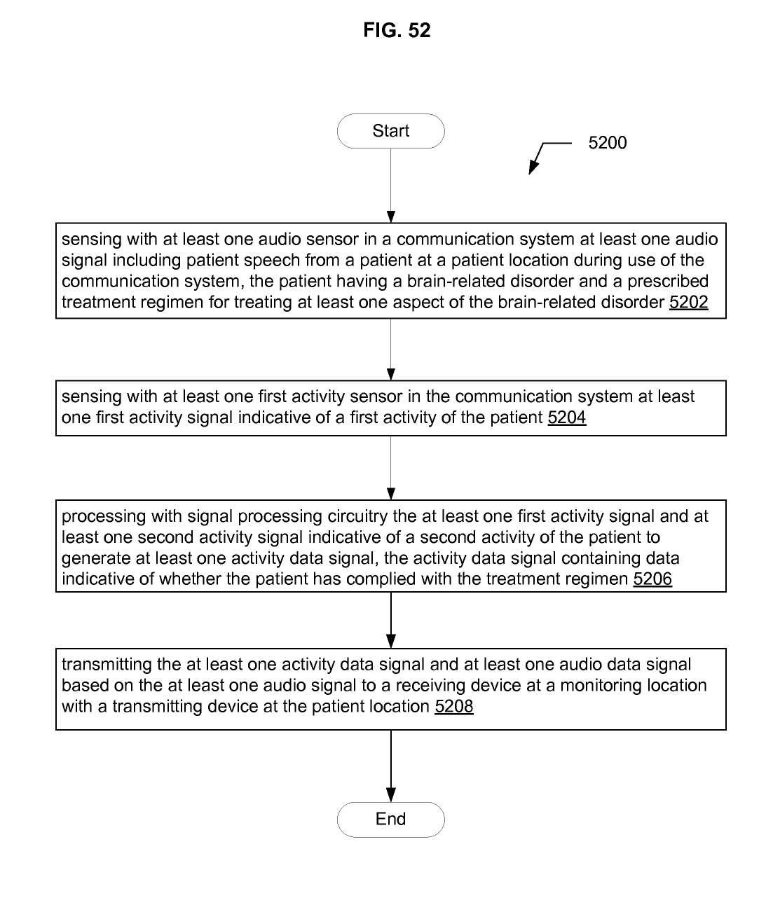

FIG. 52 is a flow diagram of a method 5200 relating to monitoring compliance of a patient with a prescribed treatment regimen using a system such as system 5102 in FIG. 51 according to principles as described herein above. Method 5200 includes sensing with at least one audio sensor in a communication system at least one audio signal including patient speech from a patient at a patient location during use of the communication system, the patient having a brain-related disorder and a prescribed treatment regimen for treating at least one aspect of the brain-related disorder, as indicated at 5202; sensing with at least one first activity sensor in the communication system at least one first activity signal indicative of a first activity of the patient, as indicated at 5204; processing with signal processing circuitry the at least one first activity signal and at least one second activity signal indicative of a second activity of the patient to generate at least one activity data signal, the activity data signal containing data indicative of whether the patient has complied with the treatment regimen, as indicated at 5206; and transmitting the at least one activity data signal and at least one audio data signal based on the at least one audio signal to a receiving device at a monitoring location with a transmitting device at the patient location, as indicated at 5208.

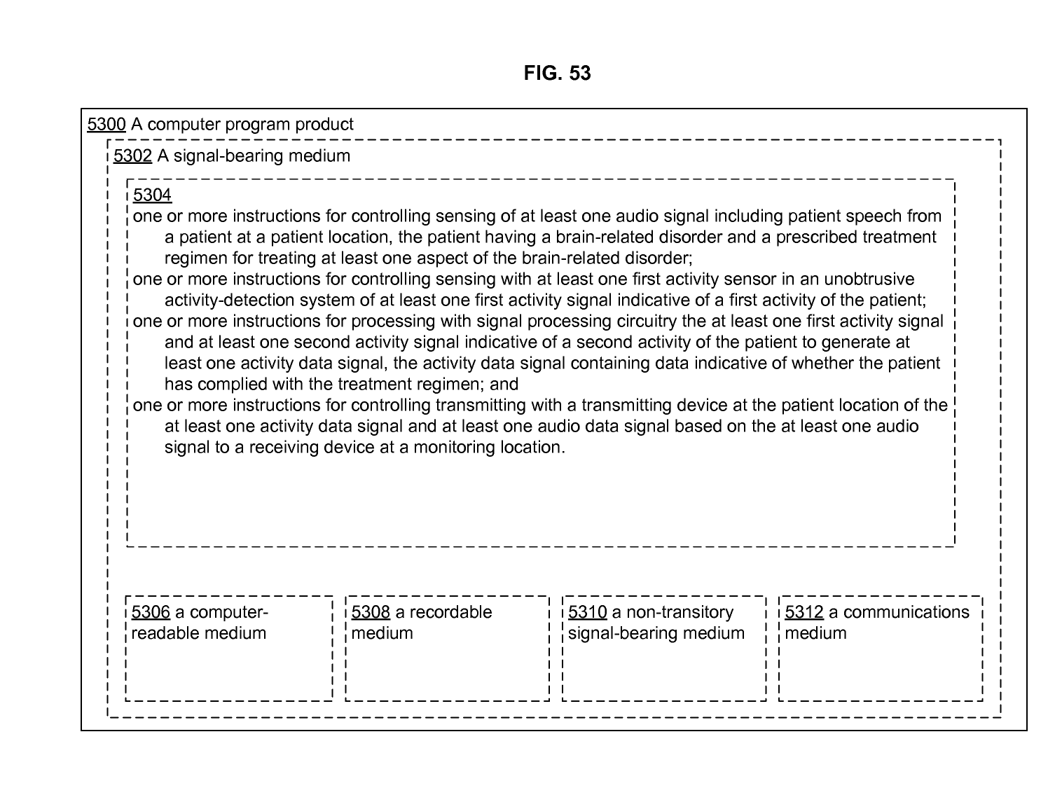

FIG. 53 is a block diagram of a computer program product 5300 for implementing a method 5200 as described in connection with FIG. 52. Computer program product 5300 includes a signal-bearing medium 5302 bearing one or more instructions for controlling sensing of at least one audio signal including patient speech from a patient at a patient location, the patient having a brain-related disorder and a prescribed treatment regimen for treating at least one aspect of the brain-related disorder; one or more instructions for controlling sensing with at least one first activity sensor in an unobtrusive activity-detection system of at least one first activity signal indicative of a first activity of the patient; one or more instructions for processing with signal processing circuitry the at least one first activity signal and at least one second activity signal indicative of a second activity of the patient to generate at least one activity data signal, the activity data signal containing data indicative of whether the patient has complied with the treatment regimen; and one or more instructions for controlling transmitting with a transmitting device at the patient location of the at least one activity data signal and at least one audio data signal based on the at least one audio signal to a receiving device at a monitoring location, as indicated at 5304. Signal-bearing medium 5302 may be, for example, a computer-readable medium 5306, a recordable medium 5308, a non-transitory signal-bearing medium 5310, or a communications medium 5312, examples of which are described herein above.

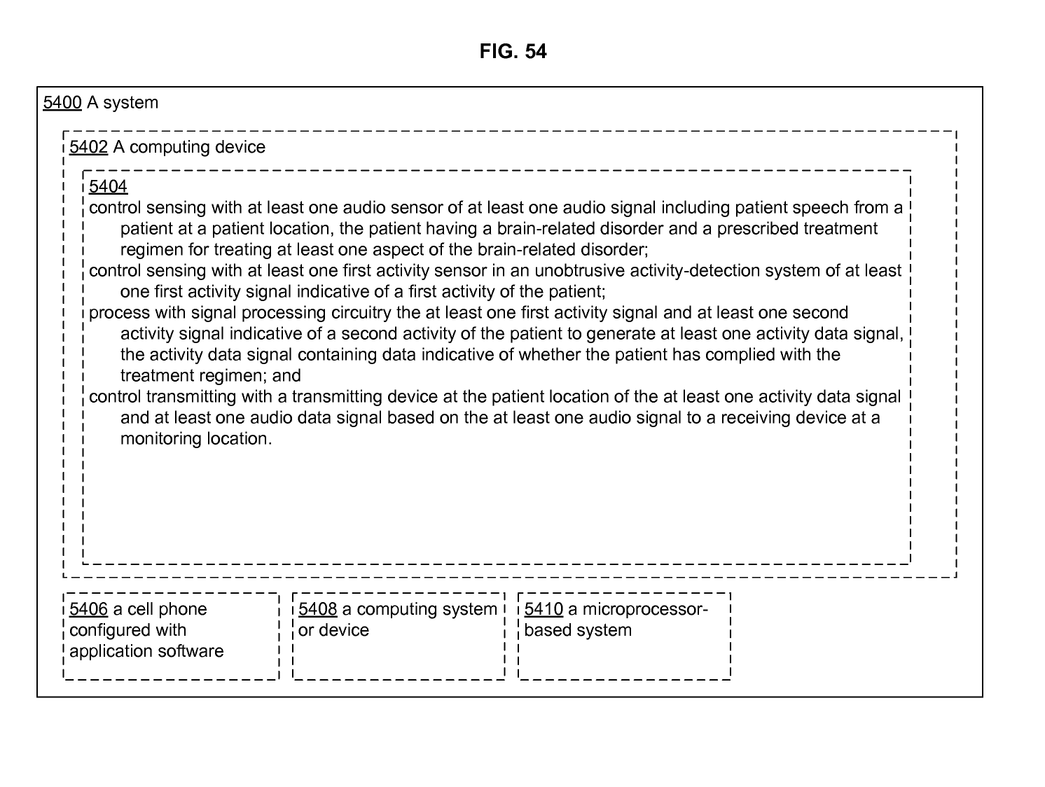

FIG. 54 is a block diagram of a system 5400 for implementing a method as described in connection with FIG. 52. System 5400 includes a computing device 5402 and instructions that when executed on the computing device cause the computing device to control sensing with at least one audio sensor of at least one audio signal including patient speech from a patient at a patient location, the patient having a brain-related disorder and a prescribed treatment regimen for treating at least one aspect of the brain-related disorder; control sensing with at least one first activity sensor in an unobtrusive activity-detection system of at least one first activity signal indicative of a first activity of the patient; process with signal processing circuitry the at least one first activity signal and at least one second activity signal indicative of a second activity of the patient to generate at least one activity data signal, the activity data signal containing data indicative of whether the patient has complied with the treatment regimen; and control transmitting with a transmitting device at the patient location of the at least one activity data signal and at least one audio data signal based on the at least one audio signal to a receiving device at a monitoring location, as indicated at 5404. System 5400 may be, for example, a cell phone configured with application software 5406, a computing system or device 5408, or a microprocessor-based system 5410.

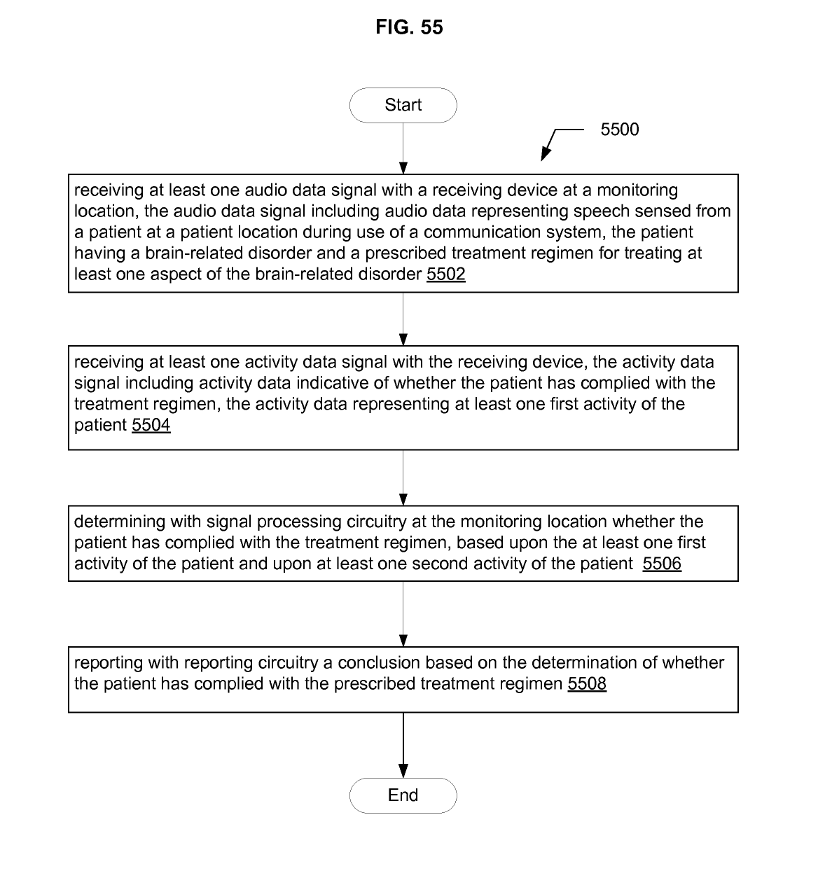

FIG. 55 is a flow diagram of a method 5500 of monitoring compliance of a patient with a treatment regimen, using a system such as monitoring system 5106 in FIG. 51. In an aspect, method 5500 includes receiving at least one audio data signal with a receiving device at a monitoring location, the audio data signal including audio data representing speech sensed from a patient at a patient location during use of a communication system, the patient having a brain-related disorder and a prescribed treatment regimen for treating at least one aspect of the brain-related disorder, as indicated at 5502; receiving at least one activity data signal with the receiving device, the activity data signal including activity data indicative of whether the patient has complied with the treatment regimen, the activity data representing at least one first activity of the patient, as indicated at 5504; determining with signal processing circuitry at the monitoring location whether the patient has complied with the treatment regimen, based upon the at least one first activity of the patient and upon at least one second activity of the patient, as indicated at 5506; and reporting with reporting circuitry a conclusion based on the determination of whether the patient has complied with the prescribed treatment regimen, as indicated at 5508.

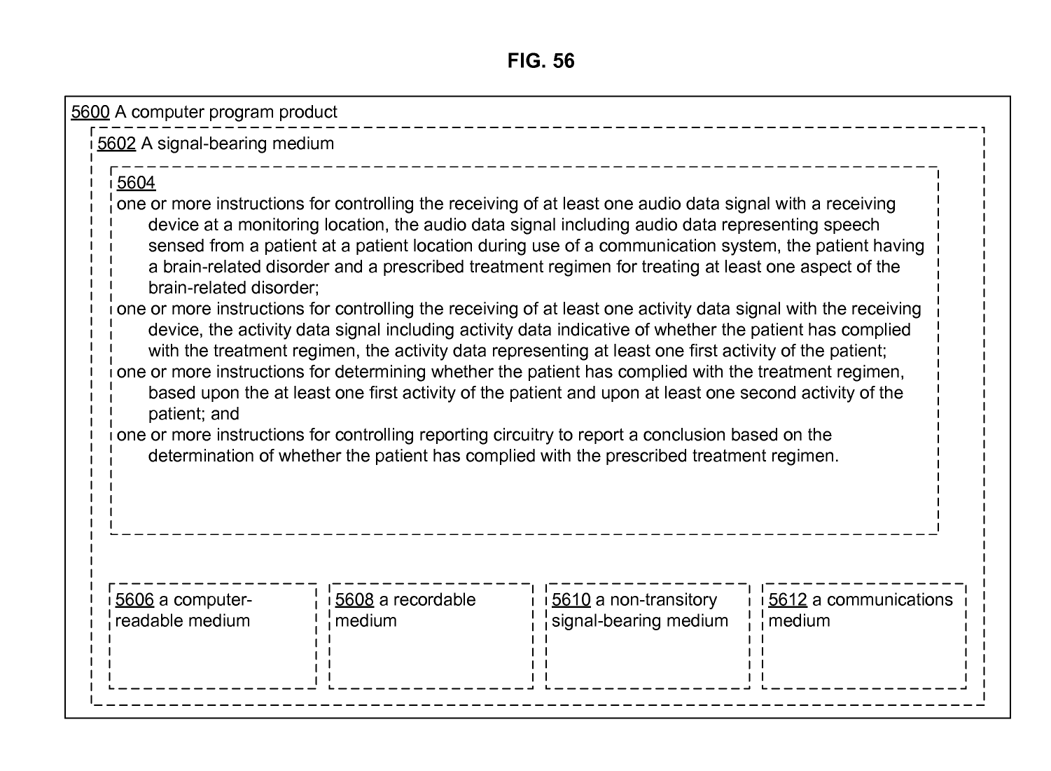

FIG. 56 is a block diagram of a computer program product 5600 for implementing a method 5500 as described in connection with FIG. 55. Computer program product 5600 includes a signal-bearing medium 5602 bearing one or more instructions for controlling the receiving of at least one audio data signal with a receiving device at a monitoring location, the audio data signal including audio data representing speech sensed from a patient at a patient location during use of a communication system, the patient having a brain-related disorder and a prescribed treatment regimen for treating at least one aspect of the brain-related disorder; one or more instructions for controlling the receiving of at least one activity data signal with the receiving device, the activity data signal including activity data indicative of whether the patient has complied with the treatment regimen, the activity data representing at least one first activity of the patient; one or more instructions for determining whether the patient has complied with the treatment regimen, based upon the at least one first activity of the patient and upon at least one second activity of the patient; and one or more instructions for controlling reporting circuitry to report a conclusion based on the determination of whether the patient has complied with the prescribed treatment regimen, as indicated at 5604. Signal-bearing medium 5602 may be, for example, a computer-readable medium 5606, a recordable medium 5608, a non-transitory signal-bearing medium 5610, or a communications medium 5612, examples of which are described herein above.

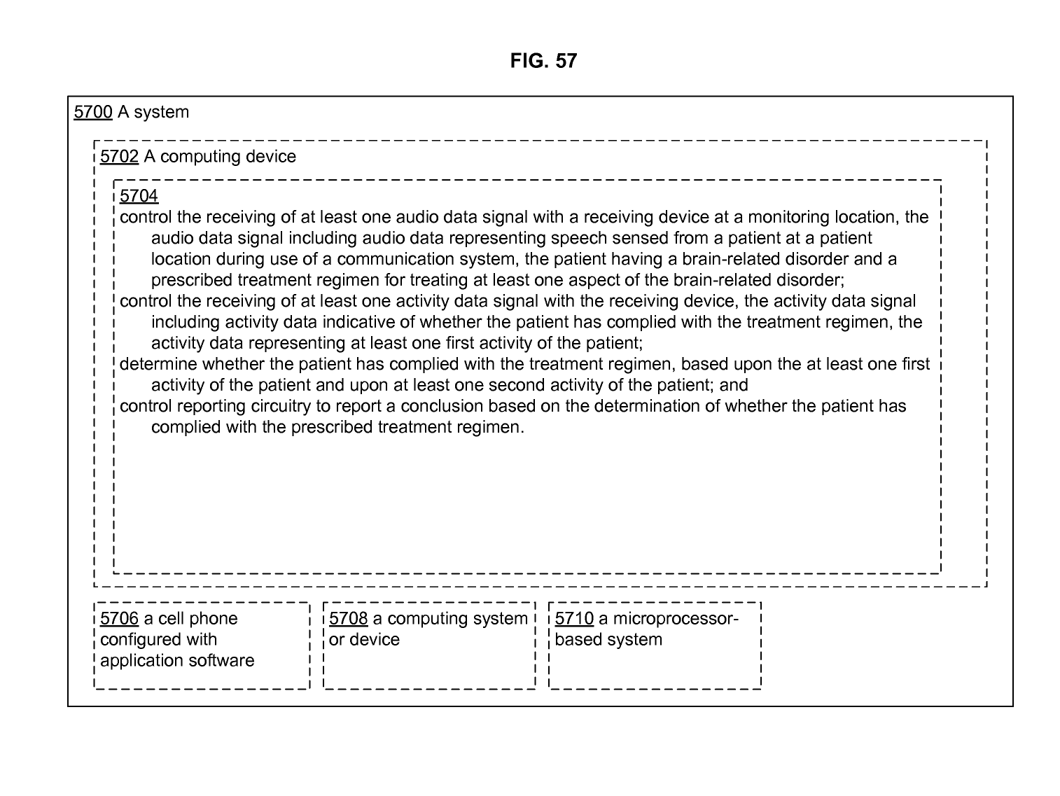

FIG. 57 is a block diagram of a system 5700 for implementing a method as described in connection with FIG. 52. System 5700 includes a computing device 5702 and instructions that when executed on the computing device cause the computing device to control the receiving of at least one audio data signal with a receiving device at a monitoring location, the audio data signal including audio data representing speech sensed from a patient at a patient location during use of a communication system, the patient having a brain-related disorder and a prescribed treatment regimen for treating at least one aspect of the brain-related disorder; control the receiving of at least one activity data signal with the receiving device, the activity data signal including activity data indicative of whether the patient has complied with the treatment regimen, the activity data representing at least one first activity of the patient; determine whether the patient has complied with the treatment regimen, based upon the at least one first activity of the patient and upon at least one second activity of the patient; and control reporting circuitry to report a conclusion based on the determination of whether the patient has complied with the prescribed treatment regimen, as indicated at 5704. System 5700 may be, for example, a cell phone configured with application software 5706, a computing system or device 5708, or a microprocessor-based system 5710.

The herein described subject matter sometimes illustrates different components contained within, or connected with, different other components. It is to be understood that such depicted architectures are merely exemplary, and that in fact many other architectures may be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components, and/or wirelessly interactable, and/or wirelessly interacting components, and/or logically interacting, and/or logically interactable components.

In some instances, one or more components may be referred to herein as “configured to,” “configured by,” “configurable to,” “operable/operative to,” “adapted/adaptable,” “able to,” “conformable/conformed to,” etc. Those skilled in the art will recognize that such terms (e.g., “configured to”) generally encompass active-state components and/or inactive-state components and/or standby-state components, unless context requires otherwise.

While particular aspects of the present subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true spirit and scope of the subject matter described herein. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although various operational flows are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

I/We claim:

1. A system comprising:
at least one audio sensor in a communication system for sensing at least one audio signal including patient speech from a patient at a patient location during use of the communication system, the patient having a brain-related disorder and a prescribed treatment regimen for treating at least one aspect of the brain-related disorder;
at least one first activity sensor for sensing at least one first activity signal indicative of a first activity of the patient;
signal processing circuitry configured to process the at least one first activity signal and at least one second activity signal indicative of a second activity of the patient to generate at least one activity data signal, the activity data signal containing activity data indicative of whether the patient has complied with the treatment regimen; and
at least one transmitting device at the patient location for transmitting the at least one activity data signal and at least one audio data signal based on the at least one audio signal to a receiving device at a monitoring location.

2. The system of claim 1, including at least one second activity sensor for sensing the at least one second activity signal indicative of the second activity of the patient.

3.-26. (canceled)

27. The system of claim 1, wherein the at least one first activity sensor is configured to transduce an activity signal associated with performance of a non-speech activity.

28.-33. (canceled)

34. The system of claim 1, wherein the first activity of the patient and the second activity of the patient are both non-speech activities.

35.-36. (canceled)

37. The system of claim 1, including
patient identification circuitry configured to determine a presence of the patient from at least one identity signal sensed at the patient location;
wherein the signal processing circuitry is configured to detect an activity of the patient based at least in part on the determination of the presence of the patient by the patient identification circuitry.

38.-55. (canceled)

56. The system of claim 1, wherein the signal processing circuitry includes activity analysis circuitry for processing the at least one first activity signal and the at least one second activity signal to determine at least one activity pattern of the patient.

57.-65. (canceled)

66. The system of claim 1, including notification circuitry for generating a notification indicative of whether the patient has complied with the treatment regimen.

67.-70. (canceled)

71. The system of claim 66, including
threat detection circuitry for determining based upon at least one of the at least one first activity signal and the at least one second activity signal that the patient poses a threat;
wherein the notification circuitry is configured to generate a notification indicative of the threat.

72. A method comprising:
sensing with at least one audio sensor in a communication system at least one audio signal including patient speech from a patient at a patient location during use of the communication system, the patient having a brain-related disorder and a prescribed treatment regimen for treating at least one aspect of the brain-related disorder;
sensing with at least one first activity sensor in the communication system at least one first activity signal indicative of a first activity of the patient;
processing with signal processing circuitry the at least one first activity signal and at least one second activity signal indicative of a second activity of the patient to generate at least one activity data signal, the activity data signal containing data indicative of whether the patient has complied with the treatment regimen; and
transmitting the at least one activity data signal and at least one audio data signal based on the at least one audio signal to a receiving device at a monitoring location with a transmitting device at the patient location.

73. The method of claim 72, including sensing the at least one second activity signal with at least one second activity sensor.

74. The method of claim 72, wherein the at least one second activity signal is the at least one audio signal.

75.-106. (canceled)

107. The method of claim 72, including
determining, with patient identification circuitry, a presence of the patient from at least one identity signal sensed at the patient location; and
detecting, with the signal processing circuitry, at least one of speech of the patient in the at least one audio signal and activity of the patient in the at least one activity signal, based at least in part on the determination of the presence of the patient by the patient identification circuitry.

108.-120. (canceled)

121. The method of claim 72, including
determining whether the patient has complied with the treatment regimen by comparing at least one of the at least one first activity signal and the at least one second activity signal with at least one characteristic activity signal;
generating the at least one activity data signal based upon the determination of whether the patient has complied with the treatment regimen.

122.-129. (canceled)

130. The method of claim 72, wherein processing with signal processing circuitry the at least one first activity signal and the at least one second activity signal includes processing at least one of the at least one first activity signal and the at least one second activity signal with pattern detection circuitry to identify at least one section of the at least one first activity signal and the at least one second activity signal containing at least one activity pattern.

131. The method of claim 130, including
including the at least one section of the at least one first activity signal and the at least one second activity signal containing at least one activity pattern in the activity data signal.

132. The method of claim 130, including
analyzing the at least one activity pattern with pattern analysis circuitry to determine at least one activity parameter for the patient.

133. The method of claim 132, including
including the at least one activity parameter in the activity data signal.

134.-140. (canceled)

141. The method of claim 130, including
comparing the at least one activity pattern with a plurality of activity patterns; and
determining which of the plurality of activity patterns best matches the at least one activity pattern.

142.-157. (canceled)

158. The method of claim 72, including
determining with threat detection circuitry in the signal processing circuitry, based upon the at least one first activity signal and the at least one second activity signal, that the patient poses a threat;
generating a notification indicative of the threat with notification circuitry; and
delivering the notification to warning circuitry configured to deliver a warning to a threatened party.

159. A system comprising:
a computing device; and
instructions that when executed on the computing device cause the computing device to:
control sensing with at least one audio sensor of at least one audio signal including patient speech from a patient at a patient location, the patient having a brain-related disorder and a prescribed treatment regimen for treating at least one aspect of the brain-related disorder;
control sensing with at least one first activity sensor in an unobtrusive activity-detection system of at least one first activity signal indicative of a first activity of the patient;
process with signal processing circuitry the at least one first activity signal and at least one second activity signal indicative of a second activity of the patient to generate at least one activity data signal, the activity data signal containing data indicative of whether the patient has complied with the treatment regimen; and
control transmitting with a transmitting device at the patient location of the at least one activity data signal and at least one audio data signal based on the at least one audio signal to a receiving device at a monitoring location.

160.-183. (canceled)

184. The system of claim 1, wherein the at least one first activity sensor includes at least one of a physiological sensor, an eye position sensor, an EEG sensor, an EEG sensor configured to detect an event-related potential, a heart rate sensor, a pupil diameter sensor, an imaging device, an entertainment device or system, a game controller, an input device, a pointing device, a touchscreen, an RFID sensor, a vehicle system, a wearable sensor, an environmental sensor, a pressure sensor, a force sensor, a capacitive sensor, an optical sensor, a motion sensor, a motion capture device, and an acceleration sensor.

185. The system of claim 37, wherein the identity signal includes an image signal from an imaging device at the patient location, and wherein the patient identification circuitry is configured to analyze the image signal to determine the identity of the patient through at least one of facial recognition and gait recognition.

186. The system of claim 37, wherein the identity signal includes at least one of a portion of the first activity signal, a portion of the at least one second activity signal, an audio signal from the audio sensor at the patient location, an image signal from an imaging device at the patient location, a biometric signal from at least one biometric sensor at the patient location, an authentication factor, a security token, a password, a digital signature, a cryptographic key, a cell phone identification code, an electronic serial number, a mobile identification number, and a system identification code, and an RFID signal.

187. The system of claim 1, wherein the at least one audio sensor, the at least one first activity sensor, the signal processing circuitry, and the at least one transmitting device are components of at least one of an unobtrusive activity detection system, a communication system, a cell phone configured with application software, a computing system or device, and an intercommunication device.

188. The system of claim 56, wherein the activity analysis circuitry includes at least one of an activity analyzer for assessing the at least one activity pattern to determine at least one activity parameter indicative of whether the patient has complied with the treatment regimen, wherein the activity data signal includes the at least one activity parameter; and a comparator for comparing the at least one activity pattern with at least one characteristic activity pattern to determine whether the patient has complied with the treatment regimen.

189. The system of claim 1, wherein the transmitting device includes a wireless transmitter, a network connection, or a communication port.

190. The system of claim 66, wherein the notification circuitry includes at least one of circuitry for generating an email notification, circuitry for generating a notification to be transmitted to a wireless device, circuitry for storing a notification in a data storage device, and circuitry for generating an audio notification.

191. The method of claim 72, including
performing at least one of sensing with the at least one audio sensor, sensing with the at least one first activity sensor, processing with the signal processing circuitry, and transmitting the at least one activity data signal and the at least one audio data signal substantially continuously, intermittently, or according to a schedule.

192. The method of claim 72, wherein sensing the at least one activity signal includes sensing an activity signal corresponding to performance of a motor activity and sensing at least one activity signal corresponding to performance of an activity of daily life.

193. The method of claim 72, wherein sensing the at least one activity signal includes sensing an activity signal corresponding to performance of a motor activity, wherein the motor activity includes at least one of typing, providing an input via a user interface device, providing an input via a touchscreen, providing an input via a pointing device, controlling a game system, controlling a vehicle system, and walking.

194. The method of claim 72, wherein sensing the at least one activity signal includes sensing at least one activity signal corresponding to performance of an activity of daily life, wherein the activity of daily life includes at least one of hygiene, washing, eating, dressing, brushing hair, brushing hair, combing hair, preparing food, interacting with another person, interacting with an animal, interacting with a machine, interacting with an electronic device, using an implement, and a grooming activity.

195. The method of claim 72, wherein sensing the at least one activity signal includes sensing at least one signal from at least one of a pressure sensor, a force sensor, a capacitive sensor, an imaging device, a motion sensor, a motion capture device, an acceleration sensor, an optical sensor, and a physiological sensor.

196. The method of claim 72, including sensing at least one physiological signal with at least one physiological sensor, wherein the at least one physiological signal includes an EEG signal, a heart rate signal, an eye position signal, or a pupil diameter signal.

197. The method of claim 107, wherein the identity signal includes an image signal received from an imaging device at the patient location, and wherein determining the presence of the patient includes analyzing the image signal to determine the presence of the patient through at least one of facial recognition and gait recognition.

198. The method of claim 107, wherein the identity signal includes at least a portion of at least one of the first activity signal, the second activity signal, the at least one audio signal, an image signal received from an imaging device at the patient location, a biometric signal from at least one biometric sensor at the patient location; and wherein determining the presence of the patient includes at least one of determining that at least a portion of the at least one of the at least one first activity signal and the at least one second activity signal matches a known activity pattern of the patient, determining that at least a portion of the at least one audio signal matches a known speech pattern of the patient, analyzing the image signal to determine the presence of the patient, and analyzing the biometric signal to determine the presence of the patient.

199. The method of claim 107, wherein the identity signal includes at least one of an authentication factor, a security token, a password, a digital signature, a cryptographic key, a cell phone identification code, an electronic serial number, a mobile identification number, a system identification code, and an RFID signal.

200. The method of claim 72, including at least one of receiving at least one instruction from the monitoring location and receiving prescription information indicative of the prescribed treatment regimen, and transmitting time data to the receiving device, the time data indicative of the time at which at least one of the at least one first activity signal and the at least one second activity signal was detected.

201. The method of claim 72, wherein transmitting the at least one activity data signal and the at least one audio data signal to the receiving device at the monitoring location includes at least one of transmitting a wireless signal, transmitting a signal via a computer network connection, and storing the at least one activity data signal and the at least one audio data signal in a data storage device.

202. The method of claim 130, including at least one of comparing the at least one activity pattern with at least one characteristic activity pattern to determine whether the patient has complied with the treatment regimen and comparing the at least one activity pattern with at least one previous activity pattern of the patient to determine whether the patient has complied with the treatment regimen.

203. The method of claim 130, including
comparing the at least one activity pattern with at least one previous activity pattern of the patient to determine whether the patient has complied with the treatment regimen;
wherein the at least one previous activity pattern is representative of at least one of an activity pattern of the patient prior to initiation of treatment of the brain-related disorder, an activity pattern of the patient after initiation of treatment of the brain-related disorder, an activity pattern of the patient during known compliance of the patient with a treatment of the brain-related disorder, and an activity pattern of the patient during treatment with a specified treatment regimen.

204. The method of claim 141, wherein the plurality of activity patterns include at least one of stored prior activity patterns of the patient, the prior activity patterns representative of activity patterns of the patient with different treatment regimens, and stored population activity patterns representative of activity patterns of populations of subjects.

205. The method of claim 141, wherein the plurality of activity patterns are stored population activity patterns representative of activity patterns of populations of subjects, including at least one of activity patterns of a population of subjects without the brain-related disorder, activity patterns of a population of untreated subjects having the brain-related disorder, activity patterns of a population of subjects having the brain-related disorder stabilized by treatment, and activity patterns of a population of subjects undergoing different treatment regimens for the brain-related disorder.

206. The method of claim 72, wherein the brain-related disorder includes at least one of an emotional disorder, a personality disorder, a mental disorder, a traumatic brain-injury-related disorder, schizophrenia, Parkinson's disease, an Autism Spectrum Disorder, Alzheimer's disease, Bipolar Disorder, and depression.
